(19) United States
(12) Patent Application Publication
(10) Pub. No.: US 2025/0170194 A1
(43) Pub. Date: May 29, 2025

(54) MICROBIAL COMPOSITIONS AND METHODS FOR PRODUCING UPGRADED PROBIOTIC ASSEMBLAGES

(71) Applicant: Solarea Bio, Inc., Cambridge, MA (US)

(72) Inventors: Gerardo V. Toledo, Hopkinton, MA (US); Eric Michael Schott, West Roxbury, MA (US); Maria Juliana Soto-Giron, Cambridge, MA (US); Alicia Eve Ballok, Natick, MA (US); Mark Charbonneau, Medford, MA (US)

(21) Appl. No.: 19/054,394

(22) Filed: Feb. 14, 2025

Related U.S. Application Data
(63) Continuation-in-part of application No. 17/555,261, filed on Dec. 17, 2021, now abandoned.
Application No. 17/555,261 is a continuation of application No. PCT/US2020/038830, filed on Jun. 19, 2020.
Application No. PCT/US2020/038830 is a continuation-in-part of application No. PCT/US2019/049823, filed on Sep. 5, 2019.
(60) Provisional application No. 62/863,722, filed on Jun. 19, 2019.
(60) Provisional application No. 62/863,762, filed on Jun. 19, 2019.

(51) Int. Cl. A61K 35/747; A23L 33/135; A23L 33/14; A61K 31/715; A61K 35/742; A61K 36/064; A61P 17/06; A61P 19/02; A61P 29/00

(52) U.S. Cl. CPC A61K 35/747; A23L 33/135; A23L 33/14; A61K 31/715; A61K 35/742; A61K 36/064; A61P 17/06; A61P 19/02; A61P 29/00

(57) ABSTRACT

The present disclosure relates to the identification of a group of microorganisms, which are relatively abundant in the microbial communities associated with fruits and vegetables typically consumed raw and therefore transient or permanent members of the human microbiota. These microbes are used to augment the effects of additional probiotic strains. The consumption of mixtures of these microbes at relevant doses will produce a beneficial effect in the host. Therapeutic methods of the disclosure involve the use of live microorganisms or metabolites derived from said microorganisms to establish a microbial composition in the mammalian host that will provide a health benefit to a mammal in need thereof.

### CROSS REFERENCE TO RELATED APPLICATIONS

This application is a continuation-in-part of U.S. patent application Ser. No. 17/555,261, filed Dec. 17, 2021, which is a continuation of international application PCT/US2020/038830, filed Jun. 19, 2020, which claims the benefit of, and priority to, U.S. Provisional Application Nos. 62/863,722, filed Jun. 19, 2019 and 62/863,762, filed Jun. 19, 2019; and international application PCT/US2019/049823, filed Sep. 5, 2019.

### SEQUENCE LISTING

The instant application contains a Sequence Listing which has been submitted electronically in XML format and is hereby incorporated herein by reference in its entirety. Said XML file, created on Feb. 14, 2025, is named SBI-008USCIP_SLx.xml and is 2,688,956 bytes in size.

### FIELD OF THE DISCLOSURE

The disclosure relates to methods and compositions useful for producing upgraded probiotic assemblages.

### BACKGROUND

Daily consumption of fresh fruits, vegetables, seeds and other plant-derived ingredients of salads and juices is recognized as part of a healthy diet and associated with weight loss, weight management and overall healthy lifestyles. The health benefits associated with fresh fruits and vegetables are thought to be derived in part from vitamins, fiber, antioxidants as well as microbial products from the fermentation of complex carbohydrates and other plant-based polymers. There has been no clear mechanistic association between microbes in whole food plant-based diets and the benefits conferred by such a diet. The role of plant-associated microbes as probiotics, capable of contributing to a subject's microbiota composition and modulating human physiology in response to a plant-based diet, has been underappreciated.

Current probiotic technology does not fully leverage the beneficial properties of these microbes. Historically, probiotic compositions have been heuristically developed according to what has previously been established, most prominently featuring strains of Lactobacilli and Bifidobacteria. As such, there is a substantial opportunity to enhance the efficacy of probiotics using rationally designed compositions that combine these probiotic strains with additional beneficial, plant-derived microbes as well as with plant-derived fibers. Several aspects of human physiology that are known in the art to benefit from microbial function are described below.

### Inflammation

Microbes play a crucial role in modulating inflammation through complex interactions with the human immune system that balance pro-inflammatory and anti-inflammatory responses. Dysbiosis, or a disruption in gut microbial community structure and function, has been linked to increased inflammation and is thought to contribute chronic inflammatory conditions such as inflammatory bowel disease (IBD), rheumatoid arthritis, as well as metabolic disorders like obesity and diabetes (Godoy-Vitorino 2024). A central feature of these conditions is increased intestinal permeability, or “leaky gut,” wherein the impaired gut barrier allows proinflammatory microbial products including lipopolysaccharides (LPS) to trigger systemic inflammation and exacerbate disease symptoms (DiVincenzo et al., 2023). These observations highlight an opportunity for novel probiotic interventions to improve gut barrier function and support the management of inflammatory diseases.

Microbial metabolites are central to the immunomodulatory effects of probiotics. For example, certain bacteria, including strains Lactobacillus and Bacillus, produce short-chain fatty acids (SCFAs) that regulate immune function by inhibiting pro-inflammatory cytokines and promoting the development of regulatory T cells (Tregs) to maintain immune balance (Mann et al., 2024). Many of these effects are mediated through free fatty acid receptor 2 (FFAR2[GPR43]). This receptor is highly expressed on immune cells, including neutrophils, macrophages, and certain T cell subsets, as well as intestinal epithelial cells (Kimura et al., 2020). FFAR2 also strengthens epithelial barrier integrity and stimulates the release of antimicrobial peptides, thereby protecting against pathogenic invasion (Liu et al., 2021). By linking microbial SCFAs to immune signaling, FFAR2 plays a pivotal role in maintaining gut immune balance and has emerged as a promising therapeutic target for inflammatory and metabolic disorders. The affinity of FFAR2 for acetate and propionate is relatively high (EC₅₀ 250-500 μM) meaning that even relatively low physiologic concentrations can activate this receptor to induce anti-inflammatory responses (Schlatter et al., 2021).

Additionally, many probiotic microorganisms produce indole derivatives, such as indole-3-acetic acid (IAA), indole-3-aldehyde (IAld), indole-3-propionic acid (IPA) and indole, that can activate the human aryl hydrocarbon receptor (AhR) with different affinities (EC₅₀ ranging from 1-100 μM) (Denison et al., 2002). This transcription factor acts within the gut, to regulate inflammation and immune tolerance (Chen et al., 2023). Microbially produced neurotransmitters GABA, L-DOPA, and serotonin all show potential anti-inflammatory effects by modulating immune cell activity and reducing pro-inflammatory cytokine levels, with GABA acting on immune receptors, L-DOPA reducing neuroinflammation through dopamine pathways, and serotonin influencing immune responses via its receptors (Strandwitz 2018).

Structurally complex exopolysaccharides (EPS) produced by these bacteria can also modulate immune responses by enhancing host expression of anti-inflammatory cytokines like IL-10 while suppressing pro-inflammatory mediators (TNF-α, IL-6) and supporting intestinal barrier integrity (Salimi & Farrokh 2023). In addition, probiotics can inhibit the growth of pathogenic bacteria that trigger barrier dysfunction and inflammation (Ghosh et al., 2020).

### Gut Barrier Function

The human gastrointestinal tract functions as a barrier that contains the abundant community of microorganisms within the gut lumen and prevents opportunistic pathogens from entering internal organs of the body (Ghosh et al., 2021). Structural components of intestinal epithelium that contribute to this barrier function include tight junction proteins, gap junctions, and adherens junction proteins. However, the function of the gut barrier is impaired in several disease states, leading to increased translocation of bacteria, endotoxins, and other inflammatory mediators into host tissues (Ghosh et al., 2021). This phenomenon is often referred to as leaky gut. Gut barrier dysfunction is associated with numerous diseases that include, but are not limited to, inflammatory bowel disease (IBD), irritable bowel syndrome (IBS), celiac disease, diabetes, obesity, atherosclerosis, food allergy, rheumatoid arthritis, environment enteric dysfunction, cancer, and multiple sclerosis (Ghosh et al., 2021; Fasano 2020).

Several classes of microbial metabolites have been shown to regulate gut barrier permeability, including short chain fatty acids (acetate, propionate, and butyrate), bacteriocins, autoinducers, vitamins (vitamin K2, menadione, vitamin B2, vitamin B6, and vitamin B9), microbial amino acids, conjugated fatty acids, indole derivatives (indole, indole-3-propionic acid, 5-hydroxyl indole, indoxyl sulfate, serotonin, N-acetyltryptophan, and melatonin), bile acid metabolites, choline metabolites, and polyamines (Ghosh et al., 2021). In addition, structural components of microbial cells can influence gut barrier function. For example, bacterial components including lipopolysaccharide can activate Toll-like receptors (TLR) and nucleotide-binding and oligomerization domain (NOD)-like receptor pathways, thereby regulating epithelial tight junction expression (Ghosh et al., 2021).

These observations indicate gut barrier dysfunction is a factor that promotes systemic inflammation and contributes to a variety of disease states, and suggest that novel compositions of microbes designed to express functions that regulate intestinal barrier function could aid in improving gut barrier integrity and managing these diseases associated with leaky gut.

### SUMMARY

The disclosure relates to probiotic compositions containing a plurality of viable bacteria and fungi isolated from fresh fruits, vegetables and fermented foods comprising at least one microbial entity classified as a bacterium or fungus. Accordingly, in one aspect, the disclosure provides a synthetic microbial consortium comprising (i) at least 1×10\hat{ }7 colony forming units of a first microbial entity comprising a probiotic microbial species, and (ii) at least 1×10\hat{ }7 colony forming units of a second microbial entity comprising a food-derived microbial isolate, wherein the synthetic microbial consortium produces a synergistic functional interaction when grown together relative to each distinct microbial entity grown in isolation under the same conditions. In certain embodiments, the first microbial entity is selected from the group consisting of: a Bacillus subtilis, a Lactiplantibacillus plantarum, a Lactobacillus acidophilus, a Lacticaseibacillus casei, a Lacticaseibacillus rhamnosus, a Lacticaseibacillus paracasei, a Lentilactobacillus buchneri, a Levilactobacillus brevis, a Bacillus licheniformis, a Weizmannia coagulans, a Saccharomyces cerevisiae, and a Saccharomyces boulardii. In certain embodiments, the second microbial entity is selected from the group consisting of: Bacillus subtilis, Lactiplantibacillus plantarum, Bacillus endophyticus, Bacillus amyloliquefaciens, Bacillus safensis, Bacillus pumilus, Bacillus paralicheniformis, Bacillus amyloliquefaciens, Bacillus axarquiensis, Bacillus megaterium, Bacillus aryabhattai, Pichia membranifaciens, Pichia kudriavzevii, Pichia terricola, Saccharomyces cerevisiae, Saccharomyces boulardii, Kluyveromyces marxianus, Talaromyces atroroseus, Debaryomyces hansenii, Candida akabanensis, Candida dosseyi, Meyerozyma guilliermondii, Lentilactobacillus buchneri, Lacticaseibacillus casei, Lactobacillus acidophilus, Leuconostoc mesenteroides, Leuconostoc pseudomesenteroides, Schleiferilactobacillus harbinensis, Pediococcus parvulus, Pediococcus ethanolidurans, Pediococcus pentosaceus, Pediococcus paracasei, Galactomyces geotrichum, Nakazawaea ishiwadae, Streptococcus thermophilus, Weisella cibaria, Kazachstania servazzii, Pichia fermentans, and Holermanniella takashimae.

In certain embodiments of the synthetic microbial consortium, the synergistic functional interaction results in a two-fold or greater increased number of colony forming units produced of the first microbial entity alone or of the first and second microbial entities combined when the microbial entities are grown together, relative to the summed amount of colony forming units produced by growing an equivalent amount of each distinct microbial entity in isolation under the same culture conditions, and wherein:
(a) the first microbial entity is Bacillus subtilis, and the second microbial entity is selected from the group consisting of: Lentilactobacillus buchneri, Pediococcus parvulus, Lacticaseibacillus casei, Pediococcus ethanolidurans, Leuconostoc mesenteroides, Lactiplantibacillus plantarum, Schleiferilactobacillus harbinensis, Pichia membranifaciens, Pichia kudriavzevii, Meyerozyma guilliermondii, Holermanniella takashimae, Lactobacillus acidophilus, Galactomyces geotrichum, Candida akabanensis, Nakazawaea ishiwadae, and Saccharomyces cerevisiae;
(b) the first microbial entity is Lacticaseibacillus casei, and the second microbial entity is selected from the group consisting of: Bacillus endophyticus, Bacillus amyloliquefaciens, Bacillus subtilis, Pichia membranifaciens, Pichia kudriavzevii, Pichia fermentans, Bacillus safensis, Bacillus pumilus, and Bacillus paralicheniformis;
(c) the first microbial entity is Lactiplantibacillus plantarum, and the second microbial entity is selected from the group consisting of: Bacillus safensis, Bacillus subtilis, Saccharomyces cerevisiae, Pichia kudriavzevii, Saccharomyces boulardii, and Kluyveromyces marxianus;
(d) the first microbial entity is Lacticaseibacillus paracasei, and the second microbial entity is selected from Kluyveromyces marxianus and Saccharomyces boulardii;
(e) the first microbial entity is Lacticaseibacillus rhamnosus, and the second microbial entity is Kluyveromyces marxianus;
(f) the first microbial entity is Lentilactobacillus buchneri, and the second microbial entity is selected from the group consisting of: Bacillus subtilis, Bacillus amyloliquefaciens, Bacillus amyloliquefaciens, Bacillus safensis, Bacillus axarquiensis, Talaromyces atroroseus, Kluyveromyces marxianus and Saccharomyces boulardii;
(g) the first microbial entity is Lentilactobacillus acidophilus, and the second microbial entity is selected from the group consisting of: Bacillus axarquiensis, Bacillus amyloliquefaciens, Bacillus subtilis, and Saccharomyces boulardii;
(h) the first microbial entity is Levilactobacillus brevis, and the second microbial entity is Holermanniella takashimae;
(i) the first microbial entity is Bacillus licheniformis, and the second microbial entity is Schleiferilactobacillus harbinensis or Pediococcus ethanolidurans;
(j) the first microbial entity is Saccharomyces cerevisiae, and the second microbial entity is selected from the group consisting of: Bacillus pumilus, Bacillus subtilis, Bacillus amyloliquefaciens Lactiplantibacillus plantarum, and Leuconostoc mesenteroides; or
(k) the first microbial entity is Saccharomyces boulardii, and the second microbial entity is selected from the group consisting of: Lentilactobacillus buchneri, Pediococcus ethanolidurans, Lactobacillus harbinensis, Lactiplantibacillus plantarum, Lactobacillus acidophilus, Exiguobacterium sp., Pediococcus paracasei, Pediococcus pentosaceus and Lacticaseibacillus paracasei.

In certain embodiments of the synthetic microbial consortium, the synergistic functional interaction is production of an increased amount of acetate when the microbial entities are grown together, relative to the summed amount of acetate produced by an equivalent amount of each microbial entity grown in isolation under the same conditions, and wherein:
(a) the first microbial entity is Bacillus subtilis, and the second microbial entity is selected from the group consisting of: Leuconostoc pseudomesenteroides, Saccharomyces boulardii, Pediococcus ethanolidurans, Pediococcus pentosaceus, Lactiplantibacillus plantarum, Pichia membranifaciens, Pichia fermentans, Talaromyces atroroseus, Debaryomyces hansenii, Candida dosseyi, Streptococcus thermophilus, and Weisella cibaria;
(b) the first microbial entity is Lacticaseibacillus casei, and the second microbial entity is selected from the group consisting of: Pichia membranifaciens, Pichia fermentans, Pichia kudriavzevii, Bacillus atrophaeus, and Bacillus licheniformis;
(c) the first microbial entity is Lacticaseibacillus paracasei, and the second microbial entity is selected from the group consisting of: Bacillus subtilis, Bacillus amyloliquefaciens, Kluyveromyces marxianus, Bacillus atrophaeus, and Saccharomyces boulardii;
(d) the first microbial entity is Lactiplantibacillus plantarum, and the second microbial entity is selected from the group consisting of: Saccharomyces bouldardii, Kluyveromyces marxianus, Bacillus subtilis, Holermanniella takashimae, Bacillus pumilus, Talaromyces atroroseus, and Pichia terricola;
(e) the first microbial entity is Lentilactobacillus buchneri, and the second microbial entity is Saccharomyces boulardii or Kluyveromyces marxianus;
(f) the first microbial entity is Lentilactobacillus acidophilus, and the second microbial entity is Talaramyces atroroseus or Bacillus amyloliquefaciens;
(g) the first microbial entity is Lacticaseibacillus rhamnosus, and the second microbial entity is Saccharomyces boulardii or Talaromyces atroroseus;
(h) the first microbial entity is Bacillus licheniformis, and the second microbial entity is Lacticaseibacillus casei;
(i) the first microbial entity is Weizmannia coagulans, and the second microbial entity is Pichia fermentans or Saccharomyces cerevisiae;
(j) the first microbial entity is Saccharomyces cerevisiae, and the second microbial entity is Lactococcus lactis or Bacillus amyloliquefaciens; or
(k) the first microbial entity is Saccharomyces boulardii, and the second microbial entity is selected from the group consisting of: Lentilactobacillus buchneri, Pediococcus ethanolidurans, Lacticaseibacillus rhamnosus, Pediococcus ethanolidurans, Lactiplantibacillus plantarum, Bacillus amyloliquefaciens, Bacillus subtilis, Bacillus amyloliquefaciens, and Bacillus aryabhattai.

In certain embodiments of the synthetic microbial consortium, the synergistic functional interaction is production of an increased amount of propionate when the microbial entities are grown together, relative to the summed amount of propionate produced by an equivalent amount of each microbial entity grown in isolation under the same conditions, and wherein:
(a) the first microbial entity is Bacillus subtilis, and the second microbial entity is selected from the group consisting of: Pediococcus pentosaceus, Pichia membranifaciens, Holermanniella takashimae, Lacticaseibacillus casei, Lentilactobacillus buchneri, Lacticaseibacillus casei, and Debaryomyces hansenii;
(b) the first microbial entity is Lacticaseibacillus casei, and the second microbial entity is Bacillus subtilis or Bacillus safensis;
(c) the first microbial entity is Lentilactobacillus buchneri, and the second microbial entity is Bacillus subtilis or Bacillus amyloliquefaciens; or
(d) the first microbial entity is Saccharomyces boulardii, and the second microbial entity is Bacillus amyloliquefaciens.

In certain embodiments of the synthetic microbial consortium, the synergistic functional interaction is production of an increased amount of indole derivatives when the microbial entities are grown together, relative to the summed amount of indole derivatives produced by an equivalent amount of each microbial entity grown in isolation under the same conditions, and wherein:
(a) the first microbial entity is Bacillus subtilis, and the second microbial entity is selected from the group consisting of: Kazachstania servazzii, Kluyveromyces marxianus, Leuconostoc mesenteroides, Pediococcus parvulus, Leuconostoc pseudomesenteroides, Lactiplantibacillus plantarum, Lactiplantibacillus casei, Lentilactobacillus buchneri, Pediococcus pentosaceus, Pichia fermentans, Pichia kudriavzevii, Galactomyces geotrichum, Clavispora sp., Holermanniella takashimae, Talaromyces atroroseus, Weisella cibaria, Debaryomyces hansenii, Candida dosseyi, Saccharomyces cerevisiae, Candida almanaticensis, and Nakazawaea ishiwadae;
(b) the first microbial entity is Lacticaseibacillus casei, and the second microbial entity is selected from the group consisting of: Bacillus licheniformis, Bacillus paralicheniformis, Bacillus endophyticus, and Bacillus subtilis;
(c) the first microbial entity is Lactiplantibacillus plantarum, and the second microbial entity is selected from the group consisting of: Bacillus licheniformis, Bacillus paralicheniformis, Bacillus mojavensis, and Bacillus subtilis;
(d) the first microbial entity is Lentilactobacillus buchneri, and the second microbial entity is Bacillus axarquiensis or Bacillus subtilis;
(e) the first microbial entity is Lentilactobacillus acidophilus, and the second microbial entity is selected from the group consisting of: Candida akabanensis, Pichia terricola, Debaryomyces hansenii, and Candida dosseyi;
(f) the first microbial entity is Levilactobacillus brevis, and the second microbial entity is Bacillus subtilis;
(g) the first microbial entity is Bacillus licheniformis, and the second microbial entity is Lacticaseibacillus casei or Lactiplantibacillus plantarum; or
(h) the first microbial entity is Saccharomyces cerevisiae, and the second microbial entity is selected from the group consisting of: Bacillus mojavensis, Bacillus subtilis, Bacillus amyloliquefaciens, and Bacillus megaterium.

In certain embodiments, the synthetic microbial consortium further comprises a prebiotic polysaccharide (e.g., oligofructose, fructooligosaccharide, or a plant extract).

In certain embodiments, the synthetic microbial consortium further comprises a cryoprotectant, wherein the cryoprotectant is present in an effective amount to extend survival of the microbial entities during storage at a cryogenic temperature.

In certain embodiments, the synthetic microbial consortium is formulated as a dietary supplement, as a solid foodstuff, as a medical food, or as a pharmaceutical composition.

In certain embodiments of the synthetic microbial consortium, the microbial entities are co-formulated in a unit dose formulated for oral administration. In certain embodiments, the unit dose comprises between 1×10⁷ and 1×10¹² cfu/dose of each of the first microbial entity and the second microbial entity.

In certain embodiments of the synthetic microbial consortium, the first microbial entity and the second microbial entity increase immune health or produce an anti-inflammatory effect in a mammalian host.

In another aspect, the disclosure provides a method for treating, preventing, reducing severity, or enabling dietary management of at least one symptom in a subject having an immune system disorder, wherein the method comprises administering an effective amount of a synthetic microbial consortium of the disclosure to the subject, thereby treating, preventing, reducing the severity, or enabling the dietary management of the at least one symptom in the subject. In certain embodiments, the immune system disorder is selected from allergic rhinitis, allergic conjunctivitis, allergic bronchial asthma, atopic eczema, anaphylaxis, insect sting, drug allergy, food allergy, asthma, eczema, ulcerative colitis, Crohn's disease, celiac disease, multiple sclerosis, psoriasis, psoriatic arthritis, and a disorder or condition associated with a pathological Th17 activity.

In another aspect, the disclosure provides a synthetic microbial consortium, comprising (i) a probiotic Bacillus subtilis, and (ii) a second microbial entity comprising a food-derived microbial isolate selected from a Saccharomyces cerevisiae, a Lacticaseibacillus casei, a Pediococcus ethanolidurans, a Pichia membranifaciens, and a Lactiplantibacillus plantarum, wherein the synthetic microbial consortium produces a synergistic functional interaction comprising production of an increased amount of acetate when the microbial entities are grown together, relative to the summed amount of acetate produced by an equivalent amount of each microbial entity grown in isolation under the same conditions, and wherein administering an effective dose of the synthetic microbial consortium to a mammalian subject produces an anti-inflammatory effect in a mammalian host.

### BRIEF DESCRIPTION OF THE DRAWINGS

These and other features, aspects, and advantages of the present disclosure will become better understood with regard to the following description, and accompanying drawings.

FIG. 1 is a schematic summarizing a human gut simulator experiment. The experiment comprises an in vitro system that represents various sections of the gastrointestinal tract. Isolates of interest are incubated in the presence of conditions that mimic particular stresses in the gastro-intestinal tract (such as low pH or bile salts), heat shock, or metformin. After incubation, surviving populations are recovered. Utilizing this system, the impact of various oral anti-diabetic therapies alone or in combination with probiotic cocktails of interest on the microbial ecosystem is tested.

FIGS. 2A-2C are bar graphs depicting synergistic interactions between Saccharomyces cerevisiae var. boulardii with other commercial or food-associated microbes. FIG. 2A depicts growth synergies where S. boulardii growth is enhanced (black bars), the added food-derived strain's growth is enhanced (white bars) or the DMA combination's growth is enhanced relative to the individual strain's combined expected growth (gray bars).

FIG. 2B depicts instances where total acetate production (gray bars) is enhanced relative to the probiotic strain (black bars) and food-derived strain (white bars) individual acetate production. FIG. 2C depicts instances where total propionate production (gray bars) is enhanced relative to the probiotic strain (black bars) and food-derived strain (white bars) individual acetate production.

FIGS. 3A-3D are bar graphs depicting synergistic interactions between Lactobacillus acidophilus La-14 with other commercial or food-associated microbes. FIG. 3A depicts growth synergies where L. acidophilus growth is enhanced (black bars), the added food-derived strain's growth is enhanced (white bars) or the DMA combination's growth is enhanced relative to the individual strain's combined expected growth (gray bars).

FIG. 3B depicts indole production synergies where total indole score (gray bars) is enhanced relative to the probiotic strain (black bars) and food-derived strain (white bars) individual indole scores. FIG. 3C depicts instances where total acetate production (gray bars) is enhanced relative to the probiotic strain (black bars) and food-derived strain (white bars) individual acetate production. FIG. 3D depicts instances where total propionate production (gray bars) is enhanced relative to the probiotic strain (black bars) and food-derived strain (white bars) individual acetate production.

FIGS. 4A-4D are bar graphs depicting synergistic interactions between Bacillus subtilis DE111 with other commercial or food-associated microbes. FIG. 4A depicts growth synergies where B. subtilis growth is enhanced (black bars), the added food-derived strain's growth is enhanced (white bars) or the DMA combination's growth is enhanced relative to the individual strain's combined expected growth (gray bars). FIG. 4B depicts indole production synergies where total indole score (gray bars) is enhanced relative to the probiotic strain (black bars) and food-derived strain (white bars) individual indole scores. FIG. 4C depicts instances where total acetate production (gray bars) is enhanced relative to the probiotic strain (black bars) and food-derived strain (white bars) individual acetate production. FIG. 4D depicts instances where total propionate production (gray bars) is enhanced relative to the probiotic strain (black bars) and food-derived strain (white bars) individual acetate production.

### DETAILED DESCRIPTION

### Definitions

Terms used in the claims and specification are defined as set forth below unless otherwise specified.

The term “ameliorating” refers to any therapeutically beneficial result in the treatment of a disease state, e.g., a metabolic disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and includes but is not limited to humans, non-human primates, canines, felines, murines, bovines, equines, and porcines.

As used herein, the term “derived from” includes microbes immediately taken from an environmental sample and also microbes isolated from an environmental source and subsequently grown in pure culture.

The term “percent identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. In some aspects, percent identity is defined with respect to a region useful for characterizing phylogenetic similarity of two or more organisms, including two or more microorganisms. Percent identity, in these circumstances can be determined by identifying such sequences within the context of a larger sequence, that can include sequences introduced by cloning or sequencing manipulations such as, e.g., primers, adapters, etc., and analyzing the percent identity in the regions of interest, without including in those analyses introduced sequences that do not inform phylogenetic similarity.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

The term “sufficient amount” means an amount sufficient to produce a desired effect, e.g., an amount sufficient to alter the microbial content of a subject's microbiota.

The term “therapeutically effective amount” is an amount that is effective to ameliorate a symptom of a disease. A therapeutically effective amount can be a “prophylactically effective amount” as prophylaxis can be considered therapy.

As used herein the term “method” refers to manners, means, techniques and procedures for accomplishing a given task including, but not limited to, those manners, means, techniques and procedures either additional to, or readily developed from additional manners, means, techniques and procedures by practitioners of the chemical, pharmacological, biological, biochemical and medical arts.

As used herein, the terms “administer”, “administering”, or “administration” refer to the placement or delivery of a composition or substance (e.g., a DMA or microbial composition described herein) onto or into a subject, e.g. orally, rectally, parenterally, or intranasally. In some embodiments, one or more persons who are not the subject (e.g., an investigator or medical worker) may administer a composition or substance to the subject. In some embodiments, the subject may administer a composition or substance to themself.

As used herein, the term “treating” includes abrogating, inhibiting substantially, slowing, or reversing the progression of a condition, substantially ameliorating clinical or aesthetical symptoms of a condition.

As used herein, the term “preventing” includes completely or substantially reducing the likelihood or occurrence or the severity of initial clinical or aesthetical symptoms of a condition.

As used herein, the term “about” includes variation of up to approximately +/−10% and that allows for functional equivalence in the product.

As used herein, the term “colony-forming unit” or “cfu” is an individual cell that is able to clone itself into an entire colony of identical cells.

As used herein all percentages are weight percent unless otherwise indicated.

As used herein, “viable organisms” are organisms that are capable of growth and multiplication. In some embodiments, viability can be assessed by numbers of colony-forming units that can be cultured. In some embodiments, viability can be assessed by other means, such as quantitative polymerase chain reaction.

The term “derived from” includes material isolated from the recited source, and materials obtained using the isolated materials (e.g., cultures of microorganisms made from microorganisms isolated from the recited source).

“Microbiota” refers to the community of microorganisms that occur (sustainably or transiently) in and on an animal or plant subject, typically a mammal such as a human, including eukaryotes, archaea, bacteria, and viruses (including bacterial viruses, i.e., phage).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

The term “subject” refers to any animal subject including humans, laboratory animals (e.g., primates, rats, mice), livestock (e.g., cows, sheep, goats, pigs, turkeys, and chickens), and household pets (e.g., dogs, cats, and rodents). In certain embodiments, the subject may be suffering from a dysbiosis, including, but not limited to, an infection due to a gastrointestinal pathogen or may be at risk of developing or transmitting to others an infection due to a gastrointestinal pathogen. In certain embodiments, the subject may have, or be at risk of developing, an immune system disorder.

The “colonization” of a host organism includes the non-transitory residence of a bacterium or other microscopic organism. As used herein, “reducing colonization” of a host subject's gastrointestinal tract (or any other microbial niche) by a pathogenic bacterium includes a reduction in the residence time of the pathogen in the gastrointestinal tract as well as a reduction in the number (or concentration) of the pathogen in the gastrointestinal tract or adhered to the luminal surface of the gastrointestinal tract. Measuring reductions of adherent pathogens may be demonstrated, e.g., by a biopsy sample, or reductions may be measured indirectly, e.g., by measuring the pathogenic burden in the stool of a mammalian host.

A “combination” of two or more bacteria includes the physical co-existence of the two bacteria, either in the same material or product or in physically connected products, as well as the temporal co-administration or co-localization of the two bacteria.

As used herein, “heterologous” designates organisms to be administered that are not naturally present in the same proportions as in the therapeutic composition as in subjects to be treated with the therapeutic composition. These can be organisms that are not normally present in individuals in need of the composition described herein, or organisms that are not present in sufficient proportion in said individuals. These organisms can comprise a synthetic composition of organisms derived from separate plant sources or can comprise a composition of organisms derived from the same plant source, or a combination thereof.

“Controlled-release” refers to delayed release of an agent, from a composition or dosage form in which the agent is released according to a desired profile in which the release occurs after a period of time.

The term “agriculturally-derived” means a microorganism isolated from an edible crop, a fermented food from such edible crop or products from animal origin in a farm setting such as dairy.

The term “organic farming” refers to an alternative agricultural system avoiding the use of agrochemicals including fertilizers and pesticides and substitutes these with compost and other natural practices. In a non-limiting illustrative example, USDA standards for organic practices require:
Land must have had no prohibited substances applied to it for at least 3 years before the harvest of an organic crop
Soil fertility and crop nutrients will be managed through tillage and cultivation practices, crop rotations, and cover crops, supplemented with animal and crop waste materials and allowed synthetic materials
Crop pests, weeds, and diseases will be controlled primarily through management practices including physical, mechanical, and biological controls. When these practices are not sufficient, a biological, botanical, or synthetic substance approved for use on the National List may be used
Operations must use organic seeds and other planting stock when available
The use of genetic engineering, ionizing radiation and sewage sludge is prohibited

The term “extract, fraction, tissue or portion of a plant” refers to the edible and non-edible components of a fresh fruit, vegetable or fermented food from which a subsample is collected for the isolation of bacteria and fungi.

The term “food” refers to edible nutritious substance that people or animals eat or drink in order to maintain life and growth.

The term “functional foods” refer to a food that has been enriched or fortified by the addition of other nutritional ingredients not found in the unenriched food item. The fortification can be done by the addition of vitamins, minerals and microorganisms (e.g., a probiotic microorganism and/or any of the microorganism described herein).

The term “medical foods” are foods that are specially formulated and intended for the dietary management of a disease that has distinctive nutritional needs that cannot be met by normal diet alone.

The term “foodstuff” as used herein, refers to a nutritional composition for oral administration that is in solid, liquid or gel form. A medical food can also be a foodstuff.

The term “probiotic microorganism” refers to a bacterium or fungus that, when consumed live, provides a health benefit to the consumer generally by improving or restoring the gut flora. Preferred probiotic microorganisms include microorganisms that are generally regarded as safe (“GRAS”), included certified GRAS microorganisms.

The term “probiotic composition” refers to compositions that include one or more probiotic microorganisms.

The term “prebiotic compounds” refer to a variety of compounds such as plant polymers, plant fibers, vitamins carbohydrate polymers found in dairy and other molecules that can act as substrates for microbial degradation and production of short chain fatty acids and other fermentable substrates.

The term “synbiotic” refers to a combination of probiotic microorganisms and prebiotic compounds in the same preparation and delivered together so the prebiotic compounds are utilized by the probiotic microorganisms.

The term “postbiotic” refers to probiotic cells that have been killed by heat or other means and are delivered to the consumer as dead cells.

“Beneficial microbially-produced compound” is any microbially-produced molecule that has a positive effect on human health and can be recognized as part of the use of “upgraded probiotic assemblages.”

The term “upgraded probiotic assemblages” refers to a combination of one or more microorganisms reported here in Table 4 and prebiotic compounds that when combined with a probiotic composition show a clear health beneficial effect.

The term “functional interaction” means the resulting effect when two or more bacteria and/or fungi growing in a culture vessel exchange substrates and products of metabolism with a resulting improved synergistic growth or production of a compound relative to the capacity of each of the bacteria and/or fungi grown alone; for example increased short chain fatty acid production measured by gas chromatography and the amount of short chain fatty acid by the co-cultured bacteria and/or fungi is greater than the sum of the short chain fatty acid production by the individual bacteria and/or fungi.

The term “artificially associated” means the use of rational approaches to combine microorganisms that do not naturally co-exists in the same sample, or where no demonstrations of spontaneous chance encounters, assemblies as consortium, and/or synergistic interactions are known or previously described.

The term “fermented probiotic composition” refers to an agricultural product that is subject to a natural process of microbial transformation to produce short chain fatty acids as part of preservation or flavor and that can be enriched with other microorganisms to enhance one of its properties.

The term “anti-inflammatory product” as used herein, refers to any substance that has an effect (either direct or indirect) on a subject in contact with the product that results in reduction of inflammation, or any detectable markers of inflammation known in the art.

The term “pro-inflammatory cytokines” as used herein, refers to small proteins that regulate the activity of blood cells such as immune system cells and are involved in the up-regulation of inflammatory reactions. Pro-inflammatory cytokines can be produced by activated macrophages or other immune cells, endothelial cells and epithelial cells.

The term “immune health” as used herein, refers to the functions and activity of the immune system and cells associated with the immune system of a healthy subject. As used herein, the term “improving immune health” refers to modulating the activity and/or function of the immune system so as to increase the immune system's ability to detect foreign antigens, pathogens, and/or abnormal cells (such as but not limited to cancer cells and infected cells), and/or refers to modulating the immune system's activity and/or function in a subject exhibiting abnormally increased immune system activity/immune response relative to healthy subjects, such as conditions or diseases related to increased inflammation (such as, but not limited to, Alzheimer's disease, cancer, asthma, heart disease, type II diabetes, rheumatoid arthritis) and/or conditions or diseases related to increased immune response (e.g., autoimmune disease).

The term “mammal” as used herein includes both humans and non-humans and include but is not limited to humans, non-human primates, canines, felines, murines, bovines, equines, and porcines.

The term “therapeutically effective amount” is an amount that is effective to ameliorate a symptom of a disease.

It must be noted that, as used in the specification and the appended claims, the singular forms “a,” “an” and “the” include plural referents unless the context clearly dictates otherwise.

As used herein “GOS” indicates one or more galacto-oligosaccharides and “FOS” indicates one or more fructo-oligosaccharide.

Throughout this application, various embodiments of this disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination or as suitable in any other described embodiment of the disclosure. Certain features described in the context of various embodiments are not to be considered essential features of those embodiments, unless the embodiment is inoperative without those elements.

The following abbreviations are used in this specification and/or Figures: ac refers to acetic acid; but refers to butyric acid; ppa refers to propionic acid. SBI refers to Solarea Bio isolate, DMA refers to defined microbial assemblage, rDNA refers to ribosomal DNA, cDNA refers to complementary DNA (i.e., a DNA product produced from RNA transcripts), ITS refers to internal transcribed spacer region of rDNA, HDAC refers to histone deacetylase, SCFA refers to short chain fatty acids, IAA refers to indole acetic acid, IPA refers to indole propionic acid, GABA refers to gamma aminobutyric acid, EPS refers to extracellular polymeric substances, LPS refers to lipopolysaccharide, IL-10 refers to Interleukin 10, IL-1β refers to Interleukin 1 Beta, IL-6 refers to Interleukin 6, IL-12 refers to Interleukin 12, IL-17 refers to Interleukin 17, IL-23 refers to Interleukin 23, TNFα refers to Tumor Necrosis Factor Alpha, IFN-γ refers to Interferon Gamma, Th17 refers to T helper 17 cells, CRP refers to C reactive protein, DMARD refers to disease modifying anti-rheumatic drug, PASI refers to psoriasis area severity index, TLR refers to Toll Like Receptor, CIA refers to collagen induced arthritis, DTH refers to delayed type hypersensitivity, IBD refers to inflammatory bowel disease, UC refers to ulcerative colitis, MS refers to multiple sclerosis, and PD refers to Parkinson's disease.

### Methods of the Disclosure

The compositions and methods disclosed herein can be used to treat various diseases and disorders in a subject. For example, the compositions disclosed herein can be used in a method of improving immune health and/or in treating or managing an immune symptom disorder or a symptom thereof, the method comprising administering the compositions a subject in need thereof. The immune system disorder can selected from, e.g., allergic rhinitis, allergic conjunctivitis, allergic bronchial asthma, atopic eczema, anaphylaxis, insect sting, drug allergy, food allergy, asthma, eczema, ulcerative colitis, Crohn's disease, celiac disease, multiple sclerosis, psoriasis, psoriatic arthritis, and a disorder or condition associated with a pathological Th17 activity.

The compositions described herein can be used in methods of improving immune health in a subject in need thereof, the method comprising administering to the subject an effective amount of the composition. In certain embodiments, the method modulates the level and/or activity of an inflammatory cytokine in a subject. In certain embodiments, the modulating the level and or activity of an inflammatory cytokine, comprises reducing the level and/or activity of at least one inflammatory cytokine selected from the group consisting of IFNγ, IL-12, TNF-α, IL-17, IL-6, IL-1β, IL-10, and combinations thereof. In certain embodiments, the level and/or activity of the at least one inflammatory cytokine is reduced in the serum or select tissue of subject after administration of the composition, medical food, dietary supplement or solid food stuff compared to a level and/or activity of the at least one inflammatory cytokine prior to administration of the composition, medical food, dietary supplement or solid food stuff. In certain embodiments, the level and/or activity of the at least one inflammatory cytokine in the serum or select tissue of a human subject after the administration of the effective amount of the composition, medical food, dietary supplement or solid food stuff. In certain embodiments, the method causes an anti-inflammatory effect in the subject. In certain embodiments, the anti-inflammatory effect is caused by the production of at least one anti-inflammatory metabolite by either the first microbial entity, the second microbial entity or both the first and the second microbial entities. In certain embodiments, the method prevents, reduces the severity of, and/or enables the dietary management of an immune system disorder, e.g., an immune system disorder selected from allergic rhinitis, allergic conjunctivitis, allergic bronchial asthma, atopic eczema, anaphylaxis, insect sting, drug allergy, food allergy, asthma, eczema, ulcerative colitis, Crohn's disease, celiac disease, multiple sclerosis, psoriasis, psoriatic arthritis, a disorder or condition associated with a pathological Th17 activity, and combinations thereof. In certain embodiments, improving immune health comprises reducing inflammation in the human subject.

The compositions disclosed herein can be used in a method for improving the efficacy of a probiotic microorganism in improving or maintaining health in a subject in need thereof (e.g., a human subject), the method comprising administering the composition to the subject.

The compositions disclosed herein can also be used in (i) a method for treating or managing Type 2 diabetes in a subject in need thereof, (ii) a method for treating or managing osteoporosis in a subject in need thereof, and (iii) is a method for improving the efficacy of a probiotic microorganism in improving or maintaining health in a subject in need thereof, the method comprising administering the composition to the subject.

Administration of the microbial composition can be accomplished orally or rectally, although administration is not limited to these methods. In some embodiments, the microbial composition is administered orally. In some embodiments, the microbial composition is delivered rectally. In some embodiments, the administration of the microbial composition occurs at regular intervals. In some embodiments, the administration occurs daily. In some embodiments, the administration occurs twice daily.

The microbial composition can be administered via typical pharmacological means, such as slurries, capsules, microcapsules, or solutions, although means of administration are not limited to these methods. In some embodiments, an enteric capsule or enteric microcapsule is used. In some embodiments the microbial composition involving the microbial composition described herein will be fresh or frozen prior to/upon application. In some embodiments, said microbial composition will be lyophilized or otherwise treated to increase stability or otherwise obtain a benefit from said treatment.

In some embodiments, the microbial composition is administered with an effective amount of an additional therapeutic agent or along with an effective drug regimen. These include, but are not limited to, metformin, methotrexate, Acarbose, Miglitol, Voglibose, Sitagliptin, Saxagliptin, Liraglutide, Semaglutide, Tirzepatide, Pioglitazone, dipeptidyl peptidase-4 (DPP4)-inhibitors, glucagon-like peptide-1 (GLP-1) receptor analogs, alpha glucosidase inhibitors, thiazolidinedione, and sodium/glucose cotransporter 2 (SGLT2) inhibitors. In another example, the microbial composition is administered along with anti-osteoporosis medications including but not limited to, bisphosphonates (e.g., alendronate, risedronate, ibandronate, zolendronate), biologics (e.g., denosumab, romosozumab), selective estrogen receptor mediators (e.g., Raloxifene), or anabolic agents (e.g., teriparatide, abaloparatide).

In some embodiments, the probiotic composition is co-administered with an effective amount of an additional therapeutic agent or along with an effective drug regimen to improve the efficacy of said therapeutic agent or drug regimen.

In some embodiments, the probiotic composition is co-administered with an effective amount of an additional therapeutic agent or along with an effective drug regimen to reduce the side effects of said therapeutic agent or drug regimen.

### Compositions of the Disclosure

In certain embodiments, compositions of the disclosure comprise probiotic compositions formulated for administration and/or consumption, with a prebiotic and any necessary or useful excipient. In other embodiments, compositions of the disclosure comprise probiotic compositions formulated for consumption without a prebiotic. Probiotic compositions of the disclosure are preferably isolated from foods normally consumed raw and isolated for cultivation. Preferably, microbes are isolated from different foods normally consumed raw, but multiple microbes from the same food source may be used.

It is additional to those of skill in the art how to identify microbial strains. Bacterial strains are commonly identified by 16S rRNA gene sequence. Fungal species can be identified by sequence of the internal transcribed space (ITS) regions of rDNA.

One of skill in the art will recognize that the 16S rRNA gene and the ITS region comprise a small portion of the overall genome, and so the sequence of the entire genome (whole genome sequence) may also be obtained and compared to additional species and/or strains.

Additionally, it is known to those skilled in the art that housekeeping conserved genes serve as reliable markers for bacterial and fungal identification by analyzing sequence homology due to their evolutionary conservation. Housekeeping genes are involved in essential and stable functions across different species. Seven example bacterial conserved genes include Protein GrpE, Ribosomal RNA small subunit methyltransferase H, Phosphoglycerate kinase, Transcription termination/antitermination protein NusA, 50S ribosomal protein L10, 50S ribosomal protein L23, and 30S ribosomal protein S8. Ten example fungal conserved genes include PAN2-PAN3 deadenylation complex subunit PAN3, p-loop containing nucleoside triphosphate hydrolase protein, NADPH-sulfite reductase, Zinc finger, RING/FYVE/PHD-type Zinc finger C2H2-type, Ribosome assembly factor mrt4, Exoribonuclease phosphorolytic domain 1, WD40-repeat-containing domain, Ribosomal protein S5 domain 2-type fold, and Heat shock protein 70 family. Exemplary reference sequences for these conserved genes are provided in SEQ ID NO: 179-768.

In certain embodiments, bacterial entities of the disclosure are identified by comparison of the 16S rRNA sequence to those of additional bacterial species, as is well understood by those of skill in the art. In certain embodiments, fungal species of the disclosure are identified based upon comparison of the ITS sequence to those of additional species (Schoch et al. PNAS 2012). In certain embodiments, microbial strains of the disclosure are identified by whole genome sequencing and subsequent comparison of the whole genome sequence to a database of additional microbial genome sequences. While microbes identified by whole genome sequence comparison, in some embodiments, are described and discussed in terms of their closest defined genetic match, as indicated by 16S rRNA sequence, it should be understood that these microbes are not identical to their closest genetic match and are novel microbial entities. This can be shown by examining the Average Nucleotide Identity (ANI) of microbial entities of interest as compared to the reference strain that most closely matches the genome of the microbial entity of interest. ANI is further discussed in Example 6.

In other embodiments, microbial entities described herein are functionally equivalent to previously described strains with homology at the 16S rRNA or ITS region. In certain embodiments, functionally equivalent bacterial strains have 95% identity at the 16S rRNA region and functionally equivalent fungal strains have 95% identity at the ITS region. In certain embodiments, functionally equivalent bacterial strains have 96% identity at the 16S rRNA region and functionally equivalent fungal strains have 96% identity at the ITS region. In certain embodiments, functionally equivalent bacterial strains have 97% identity at the 16S rRNA region and functionally equivalent fungal strains have 97% identity at the ITS region. In certain embodiments, functionally equivalent bacterial strains have 98% identity at the 16S rRNA region and functionally equivalent fungal strains have 98% identity at the ITS region. In certain embodiments, functionally equivalent bacterial strains have 99% identity at the 16S rRNA region and functionally equivalent fungal strains have 99% identity at the ITS region. In certain embodiments, functionally equivalent bacterial strains have 99.5% identity at the 16S rRNA region and functionally equivalent fungal strains have 99.5% identity at the ITS region. In certain embodiments, functionally equivalent bacterial strains have 100% identity at the 16S rRNA region and functionally equivalent fungal strains have 100% identity at the ITS region.

In certain embodiments, compositions disclosed herein comprise components derived from edible plants and can comprise a mixture of microorganisms, comprising bacteria, fungi, archaea, and/or other indigenous or exogenous microorganisms, all of which work together to form a microbial ecosystem with a role for each of its members.

In certain embodiments, the probiotic composition may include selected microorganisms and other ingredients that have been approved by the United States Food and Drug Administration (“US FDA” or “FDA”) and are Generally Recognized as Safe (“GRAS”). Accordingly, in embodiments where the probiotic composition is formulated to only include GRAS ingredients, the probiotic composition is suited for food products, food product contacting materials, and other compositions in which the probiotic composition will not contaminate a food or food product being produced. In certain embodiments, the probiotic composition may include selected microorganisms and other ingredients that have been approved by additional regulatory agencies and can also be considered to be Generally Recognized as Safe (“GRAS”).

In some embodiments, species of interest are isolated from plant-based food sources normally consumed raw. These isolated compositions of microorganisms from individual plant sources can be combined to create a new mixture of organisms. Particular species from individual plant sources can be selected and mixed with other species cultured from other plant sources, which have been similarly isolated and grown. In some embodiments, species of interest are grown in pure cultures before being prepared for consumption or administration. In some embodiments, the organisms grown in pure culture are combined to form a synthetic combination of organisms.

In certain embodiments, the bacteria are selected based upon their ability to modulate production of one or more branch chain fatty acids, short chain fatty acids, indole derivatives, neurotransmitters, and/or flavones in a mammalian gut.

In certain embodiments, microbial compositions comprise isolates that are capable of modulating production or activity of the enzymes involved in fatty acid metabolism, such as acetolactate synthase I, N-acetylglutamate synthase, acetate kinase, Acetyl-CoA synthetase, acetyl-CoA hydrolase, Glucan 1,4-alpha-glucosidase, or Bile acid symporter Acr3.

In certain embodiments, the administered microbial compositions colonize the treated mammal's digestive tract. In some embodiments, these colonizing microbes comprise bacterial assemblages present in whole food plant-based diets. In some embodiments, these colonizing microbes reduce free fatty acids absorbed into the body of a host by absorbing the free fatty acids in the gastrointestinal tract of mammals. In some embodiments, these colonizing microbes comprise genes encoding metabolic functions related to desirable health outcomes such as increased efficacy of anti-diabetic treatments, lowered BMI, lowered inflammatory metabolic indicators, etc.

Some embodiments comprise bacteria that are not completely viable but act by releasing metabolites that act in the gastro-intestinal tract of a patient promoting weight loss, increased efficacy of diabetic regimens, bone health or other desirable outcome. Some embodiments comprise a prebiotic composition derived from metabolites present in whole food plant-based materials, identified and enriched as part of the formula for oral delivery.

In certain embodiments, a composition of the disclosure comprises a bacterium and/or a yeast, e.g., a bacterium or yeast selected from Table 3 or Table 4. In certain embodiments, a composition of the disclosure comprises a microbe selected from Lactobacillus, a Bifidobacterium, and/or a Saccharomyces species. In certain embodiments, the composition comprises Lactobacillus reuteri, Lactobacillus paracasei, Lactobacillus salivarius Lactobacillus plantarum, Lactobacillus acidophilus, Lactobacillus reuteri protectis, Lactobacillus bulgaricus, Lactobacillus rhamnosus, Lactobacillus casei, Lactobacillus delbreukeii, Bifidobacterium infantis, Bifidobacterium longum, Bifidobacterium breve, Bifidobacterium bifidum, Bacillus cereus, Bacillus subtilis, Bacillus clausii, Bacillus coagulans, Clostridium butyricum, Akkermansia muciniphila, Hafnia alvei, and/or Saccharomyces boulardii.

In certain embodiments, the compositions described herein comprise a first and second microbial entity. In certain embodiments, a microbial entity of the composition (e.g., the second microbial entity) is selected from the group consisting of: a Lactobacillus sp., a Bifidobacterium sp., a Bacillus sp., and a Clostridium sp. In certain embodiments, the microbial entity (e.g., the second microbial entity) is selected from the group consisting of: Lactobacillus reuteri, Lactobacillus paracasei, Lactobacillus salivarius, Lactobacillus plantarum, Lactobacillus acidophilus, Lactobacillus reuteri protectis, Lactobacillus bulgaricus, Lactobacillus rhamnosus, Lactobacillus casei, Lactobacillus delbreukeii, Bifidobacterium infantis, Bifidobacterium longum, Bifidobacterium bifidum, Bifidobacterium breve, Streptococcus thermophilus, Escherichia coli Nissle 1917, Lactococcus lactis, Bacillus subtilis, Bacillus clausii, Bacillus coagulans, Clostridium butyricum, Akkermansia muciniphila, Hafnia alvei, and Saccharomyces boulardii.

In certain embodiments, a composition comprises a first and second microbial entity as set forth in the exemplary DMAs in Table 4.

In certain embodiments, at least one of the microbial entities in the composition (e.g., the first and/or second microbial entity) comprises a nucleic acid sequence having at least about 97% identity at the 16S or ITS locus with a sequence shown in SEQ ID NO: 1-178. In certain embodiments, at least one of the microbial entities (e.g., the first and/or second microbial entity) is selected from the species set forth in Table 3 or Table 4. In certain embodiments, the microbial entity selected from Table 3 or Table 4 comprises a nucleic acid sequence having at least 97% identity (e.g., at least 98% identity, at least 99% identity, or 100% identity) at the 16S or ITS locus with a sequence shown in SEQ ID NO: 1-178. In certain embodiments, each of the microbial entities in the composition is selected from the microbes disclosed in Table 3 or Table 4, optionally wherein each of the microbial entities comprises a nucleic acid sequence having at least 97% identity (e.g., at least 98% identity, at least 99% identity, or 100% identity) at the 16S or ITS locus with a sequence shown in SEQ ID NO: 1-178.

In certain embodiments, the first microbial entity is isolated from a food or food product. For example, the first microbial entity can be isolated from a fruit or vegetable, e.g., a pickled food or a fermented food. In certain embodiments, the first microbial entity is isolated from a raw agricultural product. In certain embodiments, the first microbial entity is isolated from edible tissues of an agricultural product, and/or from an extract, fraction, tissue, or portion of an agricultural product.

In certain embodiments, the first and/or second microbial entity is a generally regarded as safe (GRAS) probiotic microorganism or probiotic microorganism approved for human consumption. In certain embodiments, the second microbial entity is Lactobacillus plantarum or Lactobacillus brevis.

In certain embodiments, at least one of the microbial entities of the composition is selected from the group consisting of: Lactiplantibacillus plantarum, a Lactobacillus acidophilus, a Lacticaseibacillus casei, a Lacticaseibacillus rhamnosus, a Lacticaseibacillus paracasei, a Lentilactobacillus buchneri, a Levilactobacillus brevis, a Bacillus subtilis, a Bacillus licheniformis, a Weizmannia coagulans, Saccharomyces cerevisiae, and Saccharomyces cerevisiae var. boulardii (also referred to as Saccharomyces boulardii). In certain embodiments, the microbial entity is a generally regarded as safe (GRAS) probiotic microorganism, and/or the microbial entity is approved for human consumption. In certain embodiments, the microbial entity is Lactiplantibacillus plantarum. In certain embodiments, the microbial entity is Levilactobacillus brevis.

In certain embodiments, the microbial entities in the composition are isolated from a plant-based sample, and/or the microbe is selected from the group consisting of: Bacillus endophyticus, Bacillus amyloliquefaciens, Bacillus subtilis, Bacillus safensis, Bacillus pumilus, Bacillus paralicheniformis, Bacillus amyloliquefaciens, Bacillus axarquiensis, Bacillus megaterium, Bacillus aryabhattai, Pichia membranifaciens, Pichia kudriavzevii, Pichia terricola, Saccharomyces cerevisiae, Saccharomyces boulardii, Kluyveromyces marxianus, Talaromyces atroroseus, Debaryomyces hansenii, Candida akabanensis, Clavispora sp., Candida dosseyi, Meyerozyma guilliermondii, Lentilactobacillus buchneri, Lacticaseibacillus casei, Lactobacillus acidophilus, Leuconostoc mesenteroides, Leuconostoc pseudomesenteroides, Lactiplantibacillus plantarum, Schleiferilactobacillus harbinensis, Pediococcus parvulus, Pediococcus ethanolidurans, Pediococcus pentosaceus, Pediococcus paracasei, Galactomyces geotrichum, Nakazawaea ishiwadae, Streptococcus thermophilus, Weisella cibaria, Kazachstania servazzii, Pichia fermentans, and Holermanniella takashimae.

In certain embodiments, the first microbial entity is Lacticaseibacillus casei, and the second microbial entity is selected from Bacillus endophyticus, Bacillus amyloliquefaciens, Bacillus subtilis, Pichia membranifaciens, Pichia kudriavzevii, Pichia fermentans, Bacillus safensis, Bacillus pumilus, and Bacillus paralicheniformis.

In certain embodiments, the first microbial entity is Lactiplantibacillus plantarum, and the second microbial entity is selected from Bacillus safensis, Bacillus subtilis, Saccharomyces cerevisiae, Pichia kudriavzevii, Saccharomyces boulardii, and Kluyveromyces marxianus.

In certain embodiments, the first microbial entity is Lacticaseibacillus paracasei, and the second microbial entity is selected from Kluyveromyces marxianus and Saccharomyces boulardii.

In certain embodiments, the first microbial entity is Lacticaseibacillus rhamnosus, and the second microbial entity is Kluyveromyces marxianus.

In certain embodiments, the first microbial entity is Lentilactobacillus buchneri, and the second microbial entity is selected from Bacillus subtilis, Bacillus amyloliquefaciens, Bacillus amyloliquefaciens, Bacillus safensis, Bacillus axarquiensis, Talaromyces atroroseus, Kluyveromyces marxianus or Saccharomyces boulardii.

In certain embodiments, the first microbial entity is Lentilactobacillus acidophilus, and the second microbial entity is selected from Bacillus axarquiensis, Bacillus amyloliquefaciens, Bacillus subtilis, and Saccharomyces boulardii.

In certain embodiments, the first microbial entity is Levilactobacillus brevis, and the second microbial entity is Holermanniella takashimae.

In certain embodiments, the first microbial entity is Bacillus subtilis, and the second microbial entity is selected from Lentilactobacillus buchneri, Pediococcus parvulus, Lacticaseibacillus casei, Pediococcus ethanolidurans, Leuconostoc mesenteroides, Lactiplantibacillus plantarum, Schleiferilactobacillus harbinensis, Pichia membranifaciens, Pichia kudriavzevii, Meyerozyma guilliermondii, Holermanniella takashimae, Lactobacillus acidophilus, Galactomyces geotrichum, Candida akabanensis, Nakazawaea ishiwadae, and Saccharomyces cerevisiae.

In certain embodiments, the first microbial entity is Bacillus licheniformis, and the second microbial entity is Schleiferilactobacillus harbinensis or Pediococcus ethanolidurans.

In certain embodiments, the first microbial entity is Saccharomyces cerevisiae, and the second microbial entity is selected from Bacillus pumilus, Bacillus subtilis, Bacillus amyloliquefaciens Lactiplantibacillus plantarum, or Leuconostoc mesenteroides.

In certain embodiments, the first microbial entity is Saccharomyces boulardii, and the second microbial entity is selected from Lentilactobacillus buchneri, Pediococcus ethanolidurans, Lactobacillus harbinensis, Lactiplantibacillus plantarum, Lactobacillus acidophilus, Exiguobacterium sp., Pediococcus paracasei, Pediococcus pentosaceus and Lacticaseibacillus paracasei.

In certain embodiments, the first microbial entity is Lacticaseibacillus casei, and the second microbial entity is selected from Pichia membranifaciens, Pichia fermentans, Pichia kudriavzevii, Bacillus atrophaeus, and Bacillus licheniformis.

In certain embodiments, the first microbial entity is Lacticaseibacillus paracasei, and the second microbial entity is selected from Bacillus subtilis, Bacillus amyloliquefaciens, Kluyveromyces marxianus, Bacillus atrophaeus, and Saccharomyces boulardii.

In certain embodiments, the first microbial entity is Lactiplantibacillus plantarum, and the second microbial entity is selected from Saccharomyces bouldardii, Kluyveromyces marxianus, Bacillus subtilis, Holermanniella takashimae, Bacillus pumilus, Talaromyces atroroseus, and Pichia terricola.

In certain embodiments, the first microbial entity is Lentilactobacillus buchneri, and the second microbial entity is selected from Saccharomyces boulardii and Kluyveromyces marxianus.

In certain embodiments, the first microbial entity is Lentilactobacillus acidophilus, and the second microbial entity is selected from Talaramyces atroroseus and Bacillus amyloliquefaciens.

In certain embodiments, the first microbial entity is Lacticaseibacillus rhamnosus, and the second microbial entity is Saccharomyces boulardii or Talaromyces atroroseus.

In certain embodiments, the first microbial entity is Bacillus subtilis, and the second microbial entity is selected from Leuconostoc pseudomesenteroides, Saccharomyces boulardii, Pediococcus ethanolidurans, Pediococcus pentosaceus, Lactiplantibacillus plantarum, Pichia membranifaciens, Pichia fermentans, Talaromyces atroroseus, Debaryomyces hansenii, Candida dosseyi, Streptococcus thermophilus, and Weisella cibaria.

In certain embodiments, the first microbial entity is Bacillus licheniformis, and the second microbial entity is Lacticaseibacillus casei.

In certain embodiments, the first microbial entity is Weizmannia coagulans, and the second microbial entity is Pichia fermentans or Saccharomyces cerevisiae.

In certain embodiments, the first microbial entity is Saccharomyces cerevisiae, and the second microbial entity is selected from Lactococcus lactis or Bacillus amyloliquefaciens.

In certain embodiments, the first microbial entity is Saccharomyces boulardii, and the second microbial entity is selected from Lentilactobacillus buchneri, Pediococcus ethanolidurans, Lacticaseibacillus rhamnosus, Pediococcus ethanolidurans, Lactiplantibacillus plantarum, Bacillus amyloliquefaciens, Bacillus subtilis, Bacillus amyloliquefaciens, and Bacillus aryabhattai.

In certain embodiments, the first microbial entity is Lacticaseibacillus casei, and the second microbial entity is selected from Bacillus subtilis and Bacillus safensis.

In certain embodiments, the first microbial entity is Lentilactobacillus buchneri, and the second microbial entity is selected from Bacillus subtilis and Bacillus amyloliquefaciens.

In certain embodiments, the first microbial entity is Bacillus subtilis, and the second microbial entity is selected from Pediococcus pentosaceus, Pichia membranifaciens, Holermanniella takashimae, Lacticaseibacillus casei, Lentilactobacillus buchneri, Lacticaseibacillus casei, and Debaryomyces hansenii.

In certain embodiments, the first microbial entity is Saccharomyces boulardii, and the second microbial entity is Bacillus amyloliquefaciens.

In certain embodiments, the first microbial entity is Lacticaseibacillus casei, and the second microbial entity is selected from Bacillus licheniformis, Bacillus paralicheniformis, Bacillus endophyticus, and Bacillus subtilis.

In certain embodiments, the first microbial entity is Lactiplantibacillus plantarum, and the second microbial entity is selected from Bacillus licheniformis, Bacillus paralicheniformis, Bacillus mojavensis, and Bacillus subtilis.

In certain embodiments, the first microbial entity is Lentilactobacillus buchneri, and the second microbial entity is selected from Bacillus axarquiensis and Bacillus subtilis.

In certain embodiments, the first microbial entity is Lentilactobacillus acidophilus, and the second microbial entity is selected from Candida akabanensis, Pichia terricola, Debaryomyces hansenii, and Candida dosseyi.

In certain embodiments, the first microbial entity is Levilactobacillus brevis, and the second microbial entity is Bacillus subtilis.

In certain embodiments, the first microbial entity is Bacillus subtilis, and the second microbial entity is selected from Kazachstania servazzii, Kluyveromyces marxianus, Leuconostoc mesenteroides, Pediococcus parvulus, Leuconostoc pseudomesenteroides, Lactiplantibacillus plantarum, Lactiplantibacillus casei, Lentilactobacillus buchneri, Pediococcus pentosaceus, Pichia fermentans, Pichia kudriavzevii, Galactomyces geotrichum, Clavispora sp., Holermanniella takashimae, Talaromyces atroroseus, Weisella cibaria, Debaryomyces hansenii, Candida dosseyi, Saccharomyces cerevisiae, Candida almanaticensis, and Nakazawaea ishiwadae.

In certain embodiments, the first microbial entity is Bacillus licheniformis, and the second microbial entity is selected from Lacticaseibacillus casei and Lactiplantibacillus plantarum.

In some aspects, the first microbial entity is Saccharomyces cerevisiae, and the second microbial entity is selected from Bacillus mojavensis, Bacillus subtilis, Bacillus amyloliquefaciens, and Bacillus megaterium.

Also provided for herein is a probiotic assemblage comprising a first purified microbial entity isolated from a first plant-based sample, a second purified microbial entity isolated from a second plant-based sample, wherein the first purified microbial entity is artificially-associated with the second purified microbial entity, and a third microbial entity, wherein the third microbial entity is a probiotic microorganism.

Also provided for herein is a probiotic composition comprising at least one probiotic microorganism, and at least one second microbial entity purified from a plant-based sample, optionally wherein the at least one second microbial entity is a species presented in Table 3 or Table 4, and wherein the at least one probiotic microorganism and the at least one second microbial entity are capable of combining with a food product to become a functional food. In certain embodiments, the composition further comprises a prebiotic polysaccharide or a prebiotic fiber providing a carrier for the probiotic composition and to enhance the taste and flavor properties of the food product. In certain embodiments, the composition further comprises a prebiotic polysaccharide or a prebiotic fiber in dry or liquid form, wherein the prebiotic polysaccharide or the prebiotic fiber acts as a cryoprotectant. In certain embodiments, the probiotic composition can be combined with a dairy product, optionally wherein the dairy product is selected from the group consisting of: yogurt, shake, smoothie, and cheese.

In certain embodiments, the microbes in a composition of the disclosure produce more short chain fatty acids (SCFAs), indole derivative compounds, or neurotransmitters when grown together than when cultured separately, optionally wherein growth on a chosen prebiotic sugar results in increased synergy compared to growth on rich medium lacking the chosen prebiotic.

In certain embodiments, microbes in a composition of the disclosure produce more colony forming units (CFUs) when grown together than when cultured separately in the same culture medium for the same period of time.

In certain embodiments, a microbial composition of the disclosure is capable of a first functional interaction. In certain embodiments, the first functional interaction comprises synthesis of a beneficial microbially-produced compound, e.g., a short chain fatty acid or a vitamin selected from the group consisting of: acetate, propionate, butyrate, vitamin B12, K2, folate, an indole derivative, a neurotransmitter, and combinations thereof.

### Prebiotics

Prebiotics, in accordance with the teachings of this disclosure, comprise compositions that promote the growth of beneficial bacteria in the intestines. Prebiotic substances can be consumed by a relevant probiotic microorganism, or otherwise assist in keeping the relevant probiotic microorganism alive or stimulate its growth. When consumed in an effective amount, prebiotics also beneficially affect a subject's naturally-occurring gastrointestinal microflora and thereby impart health benefits apart from just nutrition. Prebiotic foods enter the colon and serve as substrate for the endogenous bacteria, thereby indirectly providing the host with energy, metabolic substrates, and essential micronutrients. The body's digestion and absorption of prebiotic foods is dependent upon bacterial metabolic activity, which salvages energy for the host from nutrients that escaped digestion and absorption in the small intestine.

Prebiotics help probiotic microorganisms flourish in the gastrointestinal tract, and accordingly, their health benefits are largely indirect. Metabolites generated by colonic fermentation by intestinal microflora, such as short-chain fatty acids, can play important functional roles in the health of the host. Prebiotics can be useful agents for enhancing the ability of intestinal microflora to provide benefits to their host.

Prebiotics, in accordance with the embodiments of this disclosure, include, without limitation, mucopolysaccharides, oligosaccharides, polysaccharides, amino acids, vitamins, nutrient precursors, proteins, and combinations thereof.

According to particular embodiments, compositions comprise a prebiotic comprising a dietary fiber, including, without limitation, polysaccharides and oligosaccharides. These compounds have the ability to increase the number of probiotic microorganisms, and augment their associated benefits. For example, an increase of beneficial Bifidobacteria likely changes the intestinal pH to support the increase of Bifidobacteria, thereby decreasing pathogenic microorganisms.

Non-limiting examples of oligosaccharides that are categorized as prebiotics in accordance with particular embodiments include galactooligosaccharides, fructooligosaccharides, inulins, isomalto-oligosaccharides, lactilol, lactosucrose, lactulose, pyrodextrins, soy oligosaccharides, transgalacto-oligosaccharides, and xylo-oligosaccharides.

According to other particular embodiments, compositions comprise a prebiotic comprising an amino acid.

Prebiotics are found naturally in a variety of foods including, without limitation, cabbage, bananas, berries, asparagus, garlic, wheat, oats, barley (and other whole grains), flaxseed, tomatoes, Jerusalem artichoke, onions and chicory, greens (e.g., dandelion greens, spinach, collard greens, chard, kale, mustard greens, turnip greens), and legumes (e.g., lentils, kidney beans, chickpeas, navy beans, white beans, black beans). Generally, according to particular embodiments, compositions comprise a prebiotic present in a sweetener composition or functional sweetened composition in an amount sufficient to promote health and wellness.

In particular embodiments, prebiotics also can be added to high-potency sweeteners or sweetened compositions. Non-limiting examples of prebiotics that can be used in this manner include fructooligosaccharides, xylooligosaccharides, galactooligosaccharides, and combinations thereof.

Many prebiotics have been discovered from dietary intake including, but not limited to: antimicrobial peptides, polyphenols, Okara (soybean pulp by product from the manufacturing of tofu), polydextrose, lactosucrose, malto-oligosaccharides, gluco-oligosaccharides (GOS), fructo-oligosaccharides (FOS), xantho-oligosaccharides, soluble dietary fiber in general. Types of soluble dietary fiber include, but are not limited to, psyllium, pectin, or inulin. Phytoestrogens (plant-derived isoflavone compounds that have estrogenic effects) have been found to have beneficial growth effects of intestinal microbiota through increasing microbial activity and microbial metabolism by increasing the blood testosterone levels, in humans and farm animals. Phytoestrogen compounds include but are not limited to: Oestradiol, Daidzein, Formononetin, Biochainin A, Genistein, and Equol.

Dosage for the compositions described herein are deemed to be “effective doses,” indicating that the probiotic or prebiotic composition is administered in a sufficient quantity to alter the physiology of a subject in a desired manner. In some embodiments, the desired alterations include reducing obesity, and or metabolic syndrome, and sequelae associated with these conditions. In some embodiments, the desired alterations are promoting rapid weight gain in livestock. In some embodiments, the prebiotic and probiotic microorganism compositions are given in addition to an anti-diabetic regimen.

In certain embodiments, the prebiotic contains a fiber and said fiber is oligofructose, or derived from a fiber source rich in oligofructose. In certain embodiments, the prebiotic is a dried fruit or vegetable powder.

In certain embodiments, the prebiotic compound is capable of being metabolized by at least one of the viable microbial entities present in the probiotic composition. In certain embodiments, the prebiotic compound is capable of being metabolized by the first or the second microbial entity. In certain embodiments, the prebiotic compound is capable of being metabolized to produce acetate, propionate, butyrate, or combinations thereof.

In certain embodiments, the composition further comprises a prebiotic polysaccharide or a prebiotic fiber. The prebiotic polysaccharide can be, for example, oligofructose or fructooligosaccharide. In certain embodiments, the prebiotic polysaccharide comprises a plant or plant extract. In certain embodiments, the prebiotic polysaccharide or the prebiotic fiber is capable of being metabolized by at least one of the microbial entities present in the probiotic composition, optionally by the first and/or the second microbial entities. In certain embodiments, the prebiotic polysaccharide or the prebiotic fiber is capable of being metabolized to produce acetate, propionate, butyrate or combinations thereof.

In certain embodiments, the prebiotic polysaccharide or the prebiotic fiber is in powdered form, and optionally provides flavorings, and wherein the prebiotic polysaccharide or the prebiotic fiber is capable of being mixed in a liquid to flavor the liquid, optionally wherein the flavor is selected from the group consisting of grape, strawberry, cranberry, blueberry, lime, lemon, and chocolate. In certain embodiments, the prebiotic polysaccharide or the prebiotic fiber comprises a plant or plant extract in the form of a juice, smoothie, shake, dry powder, tablet, granules, pellet, emulsion, paste, jelly, ferment, syrup, or brine. In some aspects, the prebiotic polysaccharide or the prebiotic fiber is derived from an agricultural product farmed using organic practices.

### Methods of Use

Combinations with Additional Probiotic Microorganisms

The disclosure relates, inter alia, to rationally-designed probiotic compositions for enhancement of probiotic microorganisms, or formulated with additional probiotic microorganisms to produce upgraded probiotic assemblages. The use of probiotics in general is additional in the art, and one of skill in the art would understand that the methods described herein could be used to improve the efficacy of diverse probiotic compositions. Additional bacteria used as probiotic microorganisms are strains of Lactobacillus and Bifidobacterium. Lactobacillus species of interest include, but are not limited to, Lactobacillus reuteri, Lactobacillus paracasei, Lactobacillus salivarius Lactobacillus plantarum, Lactobacillus acidophilus, Lactobacillus reuteri protectis, Lactobacillus bulgaricus, Lactobacillus rhamnosus, Lactobacillus casei, and Lactobacillus delbreukeii. Bifidobacterial species of interest include, but are not limited to, Bifidobacterium infantis, Bifidobacterium longum, Bifidobacterium bifidum and Bifidobacterium breve. Other probiotic microorganisms of interest include Streptococcus thermophilus, Escherichia coli Nissle, Lactococcus lactis, Bacillus subtilis, Bacillus clausii, Bacillus coagulans, Clostridium butyricum, Akkermansia muciniphila, Hafnia alvei, Saccharomyces cerevisiae and Saccharomyces cerevisiae var. boulardii (alternatively referred to as Saccharomyces boulardii).

In some embodiments, the probiotic compositions described herein are formulated with one or more of the strains above, or strains substantially similar thereto. In some embodiments, the probiotic composition described herein is formulated for consumption along with a regimen comprising one or more of the strains above, or strains substantially similar thereto.

### Timing and Dose of Probiotic Microorganisms and Prebiotics

In certain embodiments, probiotic microorganisms, such as Lactobacillus, Leuconostoc, Pediococcus, Saccharomyces, Bacillus, Pichia, Lacticaseibacillus, or Lactiplantibacillus are given prior to beginning treatment with a prebiotic compound. In an embodiment, probiotic microorganisms, such as L. mesenteroides, B. subtilis, S. cerevisiae, L. casei, P. ethanolidurans, P. membranifaciens, and/or L. plantarum are given in conjunction with treatment with a prebiotic (e.g., comprising or consisting essentially of FOS, GOS, or other appropriate polysaccharide), for part or all of the duration of treatment with the prebiotic. Thus, in an embodiment, some or all doses of a prebiotic compound (e.g., comprising or consisting essentially of FOS, GOS, or other appropriate polysaccharide) are accompanied by a dose of bacteria or fungi, e.g., live cultured bacteria or fungi, e.g., L. mesenteroides, B. subtilis, S. cerevisiae, L. casei, P. ethanolidurans, P. membranifaciens, and/or L. plantarum. In an embodiment, bacteria or fungi, e.g., L. mesenteroides, B. subtilis, S. cerevisiae, L. casei, P. ethanolidurans, P. membranifaciens, and/or L. plantarum are given initially with a prebiotic (e.g., comprising or consisting essentially of FOS, GOS, or other appropriate polysaccharide), but then use of the probiotic microorganism is discontinued. For example, the initial one, two, three, four, five, six, seven, eight, nine, ten, or more than ten days of treatment with a prebiotic (e.g., comprising or consisting essentially of FOS, GOS, or other appropriate polysaccharide) further comprises doses of bacteria or fungi, with the use of bacteria or fungi discontinued after that time. In an embodiment, bacteria or fungi, (e.g., bacteria in yogurt), or bacteria or fungi by themselves, can be given for the first two days of treatment; then the administration of bacteria or fungi is discontinued. In another embodiment, probiotic microorganisms, either alone or in combination with other substances or treatments, are used after the treatment with a prebiotic compound (comprising or consisting essentially of FOS, GOS, or other appropriate polysaccharide) is terminated. The bacteria or fungi can be taken for any suitable period after the termination of treatment with prebiotic and can be taken daily or at regular or irregular intervals. Doses can be as described below.

Any suitable amount of a probiotic microorganism per serving can be used that allows an effective microbiota in the GI as demonstrated by a reduction in weight or amelioration of other signs of metabolic syndrome measured by insulin resistance, HbA1c, body mass index (BMI), visceral adiposity, dyslipidemia, or bone mineral density. Typically, probiotic microorganisms are given as live cultured microorganisms. Herein measurement is mg indicate dry weight of purified bacteria or fungi. The dose can be about 0.001 mg to about 1 mg, or about 0.5 mg to about 5 mg, or about 1 mg to about 1000 mg, or about 2 mg to about 200 mg, or about 2 mg to about 100 mg, or about 2 mg to about 50 mg, or about 4 mg to about 25 mg, or about 5 mg to about 20 mg, or about 10 mg to about 15 mg, or about 50 mg to about 200 mg, or about 200 mg to about 1000 mg, or about 10, 11, 12, 12.5, 13, 14, or 15 mg per serving. The probiotic microorganisms can also be about 0.5% w/w to about 20% w/w of the final composition. The dose of probiotic microorganisms can be given in combination with one or more prebiotics. Another common way of specifying the amount of probiotic microorganisms is as a colony forming unit (cfu). In an embodiment, one or more strains of probiotic microorganism are ingested in an amount of about 1×10\hat{ }6 to about 1×10\hat{ }9 cfu's, or about 1×10\hat{ }6 cfu's to about 1×10\hat{ }9 cfu's, or about 10×10\hat{ }6 cfu's to about 0.5×10\hat{ }9 cfu's, or about 113×10\hat{ }5 cfu's to about 113×10\hat{ }6 cfu's, or about 240×10\hat{ }5 cfu's to about 240×10\hat{ }6 cfu's, or about 0.3×10\hat{ }9 cfu's per serving. In another embodiment, one or more strains of probiotic microorganism are administered as part of a dairy product. In an embodiment, a typical serving size for a dairy product such as fluid milk is about 240 g. In other embodiments, a serving size is about 245 g, or about 240 g to about 245 g, or about 227 to about 300 g. In an embodiment the dairy product is yogurt. Yogurt can have a serving size of about 4 oz, or about 6 oz, or about 8 oz, or about 4 oz to 10 oz, or about half cup, or about 1 cup, or about 113 g, or about 170 g, or about 227 g, or about 245 g or about 277 g, or about 100 g to about 350 g.

In an embodiment, probiotic microorganisms are given as live cultured bacteria or fungi, e.g., in combination with a prebiotic (e.g., comprising or consisting essentially of FOS, GOS, or other appropriate polysaccharide) and, optionally, other substances. The dose can be about 1 mg to about 1000 mg, or about 2 mg to about 200 mg, or about 2 mg to about 100 mg, or about 2 mg to about 50 mg, or about 4 mg to about 25 mg, or about 5 mg to about 20 mg, or about 10 mg to about 15 mg, or about 10, 11, 12, 12.5, 13, 14, or 15 mg of probiotic microorganismal cell culture dry weight. In an embodiment, as the administration of a prebiotic (e.g., comprising or consisting essentially of FOS, GOS, or other appropriate polysaccharide) dose to a subject increases, the dose of bacteria or fungi increases as well. For example, an initial dose of a prebiotic (e.g., comprising or consisting essentially of FOS, GOS, or other appropriate polysaccharides) can be about 0.6 g to 1.0 g, e.g., 0.8 g, given in combination with about 10-15 mg of probiotic microorganismal cell culture dry weight. The dose of a prebiotic (e.g., comprising or consisting essentially of FOS, GOS, or other appropriate polysaccharide) can be increased incrementally by about 0.6 g to 1.0 g, e.g., 0.8 g, and the accompanying dose of probiotic microorganismal cell culture dry weight can be increased by about 10-15 mg, e.g., about 12.5 mg.

### FOS, GOS, and Other Appropriate Polysaccharide Formulations

### A. Formulations Introduction

In an embodiment a prebiotic composition comprises inulin, FOS, lactulose, GOS, raffinose, stachyose, or a combination thereof. In addition, other plant-derived polysaccharides such as xylan, pectin, isomalto-oligosaccharides, gentio-oligosaccharides, 4-O-methyl glucuronoxylan (GX), neutral arabinoxylan (AX), heteroxylan (HX) can be combined with the probiotic microorganisms to enhance bacterial and fungal metabolic function. Some of these can be derived from plant material found in the plant host from which the probiotic microorganisms were isolated (i.e., the “cognate” plant). In some embodiments the prebiotics are thus adapted to be assimilated and digested by the accompanying probiotics in a manner that recapitulates the rich complexity and variety of polysaccharides present in the cognate plant and which play a role during digestion following its consumption of an animal.

In an embodiment a prebiotic composition comprises or consists of FOS, GOS, or other appropriate polysaccharide. In another embodiment a prebiotic composition comprises FOS, GOS, or other appropriate polysaccharide, in combination with one or more digestible saccharides. Digestible saccharides are saccharides that are digestible by humans and include, but are not limited to lactose, glucose, and galactose. In an embodiment a prebiotic composition comprises FOS, GOS, or other appropriate polysaccharide, and less than 20% weight/weight of one or more digestible saccharides (e.g. lactose, glucose, or galactose). In an embodiment a prebiotic composition comprises FOS, GOS, or other appropriate polysaccharide, and less than 10% of one or more digestible saccharides. In an embodiment a prebiotic composition comprises FOS, GOS, or other appropriate polysaccharide, and less than 5% of one or more digestible saccharides. In another embodiment a prebiotic composition contains less than 5% lactose. In another embodiment a prebiotic composition contains less than 4% lactose. In another embodiment a prebiotic composition contains less than 3% lactose. In another embodiment a prebiotic composition contains less than 2% lactose. In another embodiment a prebiotic composition contains less than 1% lactose. In another embodiment a prebiotic composition contains less than 0.5% lactose. In another embodiment a prebiotic composition contains less than 0.4% lactose. In another embodiment a prebiotic composition contains less than 0.3% lactose. In another embodiment a prebiotic composition contains less than 0.2% lactose. In another embodiment a prebiotic composition contains less than 0.1% lactose. In another embodiment a prebiotic composition contains less than 0.05% lactose. In another embodiment a prebiotic composition contains less than 0.01% lactose. In another embodiment a prebiotic composition contains less than 0.005% lactose. In an embodiment a prebiotic composition comprises FOS, GOS, or other appropriate polysaccharide, and essentially no lactose. In an embodiment a prebiotic composition does not contain any lactose. In another embodiment a prebiotic composition contains FOS, GOS, or other appropriate polysaccharide, and at least one probiotic microorganism. In another embodiment a prebiotic composition comprises FOS, GOS, or other appropriate polysaccharide, and optionally one or more of lactose, at least one probiotic microorganism, or a buffer. Additional ingredients include ingredients to improve handling, preservatives, antioxidants, flavorings and the like.

In an embodiment, a prebiotic composition comprises FOS, GOS, or other appropriate polysaccharide, or a probiotic. In other embodiment, a prebiotic composition is in the form of a powder, tablet, capsule, syrup, or liquid. In an embodiment, a prebiotic composition can be administered with a dairy product and is in the form of milk or other common dairy product such as a yogurt, shake, smoothie, cheese, and the like.

In embodiments where a prebiotic composition comprises less than 100% by weight of FOS, GOS, or other appropriate polysaccharide, the remaining ingredients can be any suitable ingredients intended for the consumption of the subject in need thereof, e.g., human, including, but not limited to, other prebiotics (e.g., FOS), a buffer, one or more digestible saccharides (e.g. lactose, glucose, or galactose), ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings, and the like.

### B. Buffer Components

One or more buffers, optionally with a calcium counter ion, can also be administered in methods and compositions described herein. Any buffer suitable for consumption by the subject being treated, e.g., human, are useful for the compositions herein. The buffer can partially or wholly neutralize stomach acidity, which can, e.g., allow live bacteria or fungi to reach the gut. Buffers include citrates, phosphates, and the like. One embodiment utilizes a buffer with a calcium counter ion, such as Calcium Phosphate Tribasic. The calcium can serve to restore the calcium that many lactose intolerant subjects are missing in their diet. Calcium phosphate can protect Lactobacillus spp. from bile.

In an embodiment, a buffer such as calcium phosphate is given prior to beginning treatment with a prebiotic composition (such as a composition comprising or consisting essentially of FOS, GOS, or other appropriate polysaccharide), optionally in conjunction with administration of bacteria or fungi. In an embodiment, a buffer such as calcium phosphate is given in conjunction with treatment with a prebiotic composition (e.g., a composition comprising or consisting essentially of FOS, GOS, or other appropriate polysaccharide), for part or all of the treatment with lactose. Thus, in an embodiment, some or all doses of a prebiotic composition are accompanied by a dose of a buffer such as calcium phosphate. In an embodiment, a buffer such as calcium phosphate is given initially with a prebiotic composition (such as a composition comprising or consisting essentially of FOS, GOS, or other appropriate polysaccharide), but then the buffer use is discontinued. For example, the initial one, two, three, four, five, six, seven, eight, nine, ten, or more than ten days of treatment with a prebiotic composition can include doses of a buffer such as calcium phosphate, with the use of the buffer discontinued after that time. In an embodiment, a buffer such as calcium phosphate can be given for the first two days of treatment, and then the administration of buffer is discontinued. In an embodiment, a buffer such as calcium phosphate, either alone or in combination with other substances or treatments is used after the treatment with a prebiotic composition is terminated. A buffer such as calcium phosphate can be taken for any suitable period after the termination of treatment with lactose, and can be taken daily or at regular or irregular intervals. Doses can be as described below.

Numerous buffers suitable for human consumption are known in the art, and any suitable buffer can be used in the methods and compositions described herein. Calcium triphosphate is an exemplary buffer, and its counterion supplies a nutrient that is often lacking in lactose-intolerant subjects, i.e., calcium. In an embodiment a buffer can be used in a dose from about 2 mg to about 2000 mg, or about 4 mg to about 400 mg, or about 4 mg to about 200 mg, or about 4 mg to about 100 mg, or about 8 mg to about 50 mg, or about 10 mg to about 40 mg, or about 20 mg to about 30 mg, or about 20, 21, 22, 23, 24, 25, 26, 27, 28, 29, or 30 mg. In another embodiment a prebiotic composition further comprises an amount of a buffer from 1-50 mg, such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg. In an embodiment, buffer is used in a dose of about 25 mg. In an embodiment, calcium phosphate is used in a dose of about 25 mg. The dose can be given in combination with a prebiotic composition (e.g., a composition comprising or consisting essentially of FOS, GOS, or other appropriate polysaccharide). In an embodiment, as a prebiotic composition dose increases, the dose of buffer increases as well. For example, an initial dose of a prebiotic composition can be about 0.6 g to 1.0 g, e.g., 0.8 g, given in combination with about 20-30 mg, e.g., about 25 mg, of buffer, e.g., calcium phosphate. The dose of a prebiotic composition can be increased incrementally by about 0.6 g to 1.0 g, e.g., 0.8 g, and the accompanying dose of buffer, e.g., calcium phosphate, can be increased by about 20-30 mg, e.g., about 25 mg, of buffer, e.g., calcium phosphate.

### C. Compositions Comprising GOS and at Least One Probiotic Microorganism

In an embodiment, a prebiotic composition comprises FOS, GOS, or other appropriate polysaccharide, and at least one probiotic microorganism. The FOS, GOS, or other appropriate polysaccharide can comprise more than 1% of the weight of the composition while the at least one probiotic microorganism will typically comprise less than about 10%, 5%, 4%, 3%, or 2% by weight of the compositions. For example, the FOS, GOS, or other appropriate polysaccharide can be present at about 1-99.75% by weight and the at least one probiotic microorganism at about 0.25-2% by weight, or the FOS, GOS, or other appropriate polysaccharide can be present at about 89-96% by weight and the bacteria or fungi at about 1.2-3.7% by weight. In an embodiment, FOS, GOS, or other appropriate polysaccharide are present at about 92% by weight and at least one probiotic microorganism, (e.g., members from Table 3 or Table 4), is present at about 1.5% by weight. In an embodiment, FOS, GOS, or other appropriate polysaccharide are present at about 92% by weight and at least one probiotic microorganism, (e.g., members from Table 3 or Table 4), is present at about 1.5% by weight. In another embodiment, FOS, GOS, or other appropriate polysaccharide are present at about 93% by weight and at least one probiotic microorganism, (e.g., members from Table 3 or Table 4), is present at about 1.5% by weight. In another embodiment, FOS, GOS, or other appropriate polysaccharide are present at about 94% by weight and at least one probiotic microorganism, (e.g., members from Table 3 or Table 4), is present at about 1.5% by weight. In another embodiment, FOS, GOS, or other appropriate polysaccharide are present at about 95% by weight and at least one probiotic microorganism, (e.g., members from Table 3 or Table 4), is present at about 1.5% by weight. In another embodiment, FOS, GOS, or other appropriate polysaccharide are present at about 96% by weight and at least one probiotic microorganism, (e.g., members from Table 3 or Table 4), is present at about 1.5% by weight. In another embodiment, FOS, GOS, or other appropriate polysaccharide are present at about 97% by weight and at least one probiotic microorganism, (e.g., members from Table 3 or Table 4), is present at about 1.5% by weight. In another embodiment, FOS, GOS, or other appropriate polysaccharide are present at about 98% by weight and at least one probiotic microorganism, (e.g., members from Table 3 or Table 4), is present at about 1.5% by weight. In another embodiment, FOS, GOS, or other appropriate polysaccharide are present at about 98.5% by weight and at least one probiotic microorganism, (e.g., members from Table 3 or Table 4), is present at about 1.5% by weight. If the at least one probiotic microorganism and FOS, GOS, or other appropriate polysaccharide do not make up 100% by weight of the prebiotic composition, the remaining ingredients can be any suitable ingredients intended for consumption by the subject in need thereof, e.g., human, including, but not limited to, other prebiotics (e.g., FOS), one or more buffers, digestible saccharides (e.g. lactose, glucose, or galactose), ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

### D. Compositions Comprising FOS, GOS, or Other Appropriate Polysaccharide and a Buffer

In another embodiment, a prebiotic composition comprises FOS, GOS, or other appropriate polysaccharide and a buffer (e.g., calcium phosphate tribasic). For example, FOS, GOS, or other appropriate polysaccharide can be present at about 1-100% by weight and the buffer at about 0.50-4% by weight, or FOS, GOS, or other appropriate polysaccharide can be present at about 1-96% by weight and the buffer at about 1 to about 3.75% by weight. In an embodiment, FOS, GOS, or other appropriate polysaccharide are present at about 1% by weight and buffer is present at about 3% by weight. In an embodiment, FOS, GOS, or other appropriate polysaccharide are present at about 5% by weight and buffer is present at about 3% by weight. In an embodiment, FOS, GOS, or other appropriate polysaccharide are present at about 10% by weight and buffer is present at about 3% by weight. In an embodiment, FOS, GOS, or other appropriate polysaccharide are present at about 15% by weight and buffer is present at about 15% by weight. In an embodiment, FOS, GOS, or other appropriate polysaccharide are present at about 20% by weight and buffer is present at about 3% by weight. In an embodiment, FOS, GOS, or other appropriate polysaccharide are present at about 25% by weight and buffer is present at about 3% by weight. In an embodiment, FOS, GOS, or other appropriate polysaccharide are present at about 30% by weight and buffer is present at about 3% by weight. In an embodiment, FOS, GOS, or other appropriate polysaccharide are present at about 35% by weight and buffer is present at about 3% by weight. In an embodiment, FOS, GOS, or other appropriate polysaccharide are present at about 40% by weight and buffer is present at about 3% by weight. In an embodiment, FOS, GOS, or other appropriate polysaccharide are present at about 50% by weight and buffer is present at about 3% by weight. In an embodiment, FOS, GOS, or other appropriate polysaccharide are present at about 60% by weight and buffer is present at about 3% by weight. In an embodiment, FOS, GOS, or other appropriate polysaccharide are present at about 70% by weight and buffer is present at about 3% by weight. In another embodiment, FOS, GOS, or other appropriate polysaccharide are present at about 90% by weight and buffer is present at about 3% by weight. In another embodiment, FOS, GOS, or other appropriate polysaccharide are present at about 92% by weight and buffer is present at about 3% by weight. In another embodiment, FOS, GOS, or other appropriate polysaccharide are present at about 93% by weight and buffer is present at about 3% by weight. In another embodiment, FOS, GOS, or other appropriate polysaccharide are present at about 94% by weight and buffer is present at about 3% by weight. In another embodiment, FOS, GOS, or other appropriate polysaccharide are present at about 95% by weight and buffer is present at about 3% by weight. In another embodiment, FOS, GOS, or other appropriate polysaccharide are present at about 96% by weight and buffer is present at about 3% by weight. In another embodiment, FOS, GOS, or other appropriate polysaccharide are present at about 97% by weight and buffer is present at about 2% by weight. In another embodiment, FOS, GOS, or other appropriate polysaccharide are present at about 98% by weight and buffer is present at about 1% by weight. In another embodiment, FOS, GOS, or other appropriate polysaccharide are present at about 99% by weight and buffer is present at about 1% by weight. In another embodiment, FOS, GOS, or other appropriate polysaccharide are present at about 100% by weight and buffer is present at less than about 1% by weight. If the buffer and FOS, GOS, or other appropriate polysaccharide do not make up 100% by weight of the composition, the remaining ingredients can be any suitable ingredients intended for consumption by the subject (e.g., a human) including, but not limited to, probiotic microorganisms (e.g., beneficial bacteria) or other prebiotics (e.g., FOS), but also including ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

E. Compositions Comprising a Digestible Saccharide, a Probiotic Microorganism, and FOS, GOS, or Other Appropriate Polysaccharide

In an embodiment, a prebiotic composition comprises a digestible saccharide (e.g. lactose, glucose, or galactose), a probiotic microorganism (e.g., members from Table 3 or Table 4), and FOS, GOS, or other appropriate polysaccharide. In an embodiment, lactose can be present at about 1-20% by weight, bacteria or fungi at about 0.25-20.10% by weight, and FOS, GOS, or other appropriate polysaccharide at about 1-98.75% by weight. In another embodiment lactose can be present at about 5-20% by weight, bacteria or fungi at about 0.91-1.95% by weight, and FOS, GOS, or other appropriate polysaccharide at about 1 to about 96% by weight. In another embodiment, lactose is present at about 20% by weight, bacteria or fungi at about 1.5% by weight, and FOS, GOS, or other appropriate polysaccharide are present at about 1% by weight. In another embodiment, lactose is present at about 20% by weight, bacteria or fungi at about 1.5% by weight, and FOS, GOS, or other appropriate polysaccharide are present at about 50% by weight. In another embodiment, lactose is present at about 20% by weight, bacteria or fungi at about 1.5% by weight, and FOS, GOS, or other appropriate polysaccharide are present at about 60% by weight. In another embodiment, lactose is present at about 20% by weight, bacteria or fungi at about 1.5% by weight, and FOS, GOS, or other appropriate polysaccharide are present at about 70% by weight. In another embodiment, lactose is present at about 5% by weight, bacteria or fungi at about 1.5% by weight, and FOS, GOS, or other appropriate polysaccharide are present at about 90% by weight. In another embodiment, lactose is present at about 5% by weight, bacteria or fungi at about 1.5% by weight, and FOS, GOS, or other appropriate polysaccharide are present at about 92% by weight. In another embodiment, lactose is present at about 5% by weight, bacteria or fungi at about 1.5% by weight, and FOS, GOS, or other appropriate polysaccharide are present at about 93% by weight. In another embodiment, lactose is present at about 5% by weight, bacteria or fungi at about 1% by weight, and FOS, GOS, or other appropriate polysaccharide are present at about 94% by weight. In another embodiment, lactose is present at about 4.5% by weight, bacteria or fungi at about 1.5% by weight, and FOS, GOS, or other appropriate polysaccharide are present at about 94% by weight. In another embodiment, lactose is present at about 4.5% by weight, bacteria or fungi at about 0.5% by weight, and FOS, GOS, or other appropriate polysaccharide are present at about 95% by weight. In another embodiment, lactose is present at about 3.5% by weight, bacteria or fungi at about 0.5% by weight, and FOS, GOS, or other appropriate polysaccharide are present at about 96% by weight. In another embodiment, lactose is present at about 2.5% by weight, bacteria or fungi at about 0.5% by weight, and FOS, GOS, or other appropriate polysaccharides are present at about 97% by weight. In another embodiment, lactose is present at about 1.5% by weight, bacteria or fungi at about 0.5% by weight, and FOS, GOS, or other appropriate polysaccharide are present at about 98% by weight. In another embodiment, lactose is present at about 0.5% by weight, bacteria or fungi at about 0.5% by weight, and FOS, GOS, or other appropriate polysaccharide are present at about 99% by weight. If the bacteria or fungi, FOS, GOS, or other appropriate polysaccharide and lactose do not make up 100% of the composition, the remaining ingredients can be any suitable ingredients intended for consumption by the subject, e.g., a human, including, but not limited to a buffer, digestible saccharides (e.g., lactose, glucose, or galactose), ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

### F. Compositions Comprising FOS, GOS, or Other Appropriate Polysaccharide, a Probiotic Microorganism, and Buffer

In an embodiment, a prebiotic composition comprises FOS, GOS, or other appropriate polysaccharide, a probiotic microorganism, and buffer. In an embodiment, FOS, GOS, or other appropriate polysaccharide can be present at about 1-100% by weight, a probiotic microorganism at about 0.25-2% by weight, and the buffer at about 0.50-4% by weight. In another embodiment, FOS, GOS, or other appropriate polysaccharide can be present at about 1-95% by weight, a probiotic microorganism at about 0.91-1.95% by weight, and the buffer at about 1.2-30.75% by weight. In another embodiment, FOS, GOS, or other appropriate polysaccharide are present at about 1% by weight, a probiotic microorganism at about 1.5% by weight, and buffer is present at about 3% by weight. In another embodiment, FOS, GOS, or other appropriate polysaccharide are present at about 5% by weight, a probiotic microorganism at about 1.5% by weight, and buffer is present at about 3% by weight. In another embodiment, FOS, GOS, or other appropriate polysaccharide are present at about 10% by weight, a probiotic microorganism at about 1.5% by weight, and buffer is present at about 3% by weight. In another embodiment, FOS, GOS, or other appropriate polysaccharide are present at about 15% by weight, a probiotic microorganism at about 1.5% by weight, and buffer is present at about 3% by weight. In another embodiment, FOS, GOS, or other appropriate polysaccharide are present at about 20% by weight, a probiotic microorganism at about 1.5% by weight, and buffer is present at about 3% by weight. In another embodiment, FOS, GOS, or other appropriate polysaccharide are present at about 25% by weight, a probiotic microorganism at about 1.5% by weight, and buffer is present at about 3% by weight. In another embodiment, FOS, GOS, or other appropriate polysaccharide are present at about 30% by weight, a probiotic microorganism at about 1.5% by weight, and buffer is present at about 3% by weight. In another embodiment, FOS, GOS, or other appropriate polysaccharide are present at about 35% by weight, a probiotic microorganism at about 1.5% by weight, and buffer is present at about 3% by weight. In another embodiment, FOS, GOS, or other appropriate polysaccharide are present at about 40% by weight, a probiotic microorganism at about 1.5% by weight, and buffer is present at about 3% by weight. In another embodiment, FOS, GOS, or other appropriate polysaccharide are present at about 50% by weight, a probiotic microorganism at about 1.5% by weight, and buffer is present at about 3% by weight. In another embodiment, FOS, GOS, or other appropriate polysaccharide are present at about 60% by weight, a probiotic microorganism at about 1.5% by weight, and buffer is present at about 3% by weight. In another embodiment, FOS, GOS, or other appropriate polysaccharide are present at about 70% by weight, a probiotic microorganism at about 1.5% by weight, and buffer is present at about 3% by weight. In another embodiment, FOS, GOS, or other appropriate polysaccharide are present at about 90% by weight, a probiotic microorganism at about 1.5% by weight, and buffer is present at about 3% by weight. In another embodiment, FOS, GOS, or other appropriate polysaccharide are present at about 92% by weight, a probiotic microorganism at about 1.5% by weight, and buffer is present at about 3% by weight. In another embodiment, FOS, GOS, or other appropriate polysaccharide are present at about 93% by weight, a probiotic microorganism at about 1.5% by weight, and buffer is present at about 3% by weight. In another embodiment, FOS, GOS, or other appropriate polysaccharide are present at about 94% by weight, a probiotic microorganism at about 1.5% by weight, and buffer is present at about 3% by weight. In another embodiment, FOS, GOS, or other appropriate polysaccharide are present at about 95% by weight, a probiotic microorganism at about 1.5% by weight, and buffer is present at about 3% by weight. In another embodiment, FOS, GOS, or other appropriate polysaccharide are present at about 96% by weight, a probiotic microorganism at about 1.5% by weight, and buffer is present at about 2% by weight. In another embodiment, FOS, GOS, or other appropriate polysaccharide are present at about 97% by weight, a probiotic microorganism at about 1.5% by weight, and buffer is present at about 1.5% by weight. In another embodiment, FOS, GOS, or other appropriate polysaccharide are present at about 99% by weight, a probiotic microorganism at about 0.5% by weight, and buffer is present at about 0.5% by weight. In another embodiment, FOS, GOS, or other appropriate polysaccharide are present at about 100% by weight, a probiotic microorganism at less than about 0.5% by weight, and buffer is present at less than about 0.5% by weight. If the probiotic microorganism, buffer, and FOS, GOS, or other appropriate polysaccharide do not make up 100% of the composition, the remaining ingredients can be any suitable ingredients intended for the consumption of a subject (e.g., human) including, but not limited to, other prebiotics (e.g., FOS), digestible saccharides (e.g., lactose, glucose or galactose), ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

### G. Compositions Comprising a Digestible Saccharide, FOS, GOS, or Other Appropriate Polysaccharide, and a Buffer

In an embodiment, a prebiotic composition comprises a digestible saccharide (e.g. lactose, glucose, or galactose), FOS, GOS, or other appropriate polysaccharide, and a buffer. For example, lactose can be present at about 1-20% by weight, FOS, GOS, or other appropriate polysaccharide at about 1-100% by weight, and the buffer at about 0.50-4% by weight, or the lactose can be present at about 5-20% by weight, FOS, GOS, or other appropriate polysaccharide at about 1-96% by weight, and the buffer at about 1.2-30.75% by weight. In an embodiment, lactose is present at about 20% by weight, FOS, GOS, or other appropriate polysaccharide at about 1% by weight, and buffer is present at about 3% by weight. In an embodiment, lactose is present at about 5% by weight, FOS, GOS, or other appropriate polysaccharide at about 1% by weight, and buffer is present at about 3% by weight. In an embodiment, lactose is present at about 20% by weight, FOS, GOS, or other appropriate polysaccharide at about 10% by weight, and buffer is present at about 3% by weight. In an embodiment, lactose is present at about 20% by weight, FOS, GOS, or other appropriate polysaccharide at about 15% by weight, and buffer is present at about 3% by weight. In an embodiment, lactose is present at about 20% by weight, FOS, GOS, or other appropriate polysaccharide at about 20% by weight, and buffer is present at about 3% by weight. In an embodiment, lactose is present at about 20% by weight, FOS, GOS, or other appropriate polysaccharide at about 25% by weight, and buffer is present at about 3% by weight. In an embodiment, lactose is present at about 20% by weight, FOS, GOS, or other appropriate polysaccharide at about 30% by weight, and buffer is present at about 3% by weight. In an embodiment, lactose is present at about 20% by weight, FOS, GOS, or other appropriate polysaccharide at about 35% by weight, and buffer is present at about 3% by weight. In an embodiment, lactose is present at about 20% by weight, FOS, GOS, or other appropriate polysaccharide at about 40% by weight, and buffer is present at about 3% by weight. In an embodiment, lactose is present at about 20% by weight, FOS, GOS, or other appropriate polysaccharide at about 50% by weight, and buffer is present at about 3% by weight. In an embodiment, lactose is present at about 20% by weight, FOS, GOS, or other appropriate polysaccharide at about 60% by weight, and buffer is present at about 3% by weight. In an embodiment, lactose is present at about 20% by weight, FOS, GOS, or other appropriate polysaccharide at about 70% by weight, and buffer is present at about 3% by weight. In another embodiment, lactose is present at about 5% by weight, FOS, GOS, or other appropriate polysaccharide at about 90% by weight, and buffer is present at about 3% by weight. In another embodiment, lactose is present at about 5% by weight, FOS, GOS, or other appropriate polysaccharide at about 92% by weight, and buffer is present at about 3% by weight. In another embodiment, lactose is present at about 4% by weight, FOS, GOS, or other appropriate polysaccharide at about 93% by weight, and buffer is present at about 3% by weight. In another embodiment, lactose is present at about 3% by weight, FOS, GOS, or other appropriate polysaccharide at about 94% by weight, and buffer is present at about 3% by weight. In another embodiment, lactose is present at about 2% by weight, FOS, GOS, or other appropriate polysaccharide at about 95% by weight, and buffer is present at about 3% by weight. In another embodiment, lactose is present at about 1% by weight, FOS, GOS, or other appropriate polysaccharide at about 96% by weight, and buffer is present at about 3% by weight. If a suitable prebiotic, buffer and lactose do not make up 100% of the composition by weight, the remaining ingredients can be any suitable ingredients intended for consumption by a subject (e.g., human) including, but not limited to, bacteria or fungi, ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

### H. Compositions Comprising a Digestible Saccharide, Bacteria, Fungi, GOS, and a Buffer

In an embodiment, a composition comprises a digestible saccharide (e.g. lactose, glucose, or galactose), bacteria or fungi, FOS, GOS, or other appropriate polysaccharide, and buffer. For example, lactose can be present at about 1-20% by weight, bacteria or fungi at about 0.25-2.10% by weight, FOS, GOS, or other appropriate polysaccharide at about 1-100% by weight, and the buffer at about 0.50-4% by weight, or the lactose can be present at about 5-20% by weight, bacteria or fungi at about 0.91-1.95% by weight, FOS, GOS, or other appropriate polysaccharide at about 70-95% by weight, and the buffer at about 1.2-30.75% by weight. In an embodiment, lactose is present at about 20% by weight, bacteria or fungi at about 1.47% by weight, FOS, GOS, or other appropriate polysaccharide at about 1% by weight, and buffer is present at about 3% by weight. In an embodiment, lactose is present at about 20% by weight, bacteria or fungi at about 1.47% by weight, FOS, GOS, or other appropriate polysaccharide at about 10% by weight, and buffer is present at about 3% by weight. In an embodiment, lactose is present at about 20% by weight, bacteria or fungi at about 1.47% by weight, FOS, GOS, or other appropriate polysaccharide at about 15% by weight, and buffer is present at about 3% by weight. In an embodiment, lactose is present at about 20% by weight, bacteria or fungi at about 1.47% by weight, FOS, GOS, or other appropriate polysaccharide at about 20% by weight, and buffer is present at about 3% by weight. In an embodiment, lactose is present at about 20% by weight, bacteria or fungi at about 1.47% by weight, FOS, GOS, or other appropriate polysaccharide at about 25% by weight, and buffer is present at about 3% by weight. In an embodiment, lactose is present at about 20% by weight, bacteria or fungi at about 1.47% by weight, FOS, GOS, or other appropriate polysaccharide at about 30% by weight, and buffer is present at about 3% by weight. In an embodiment, lactose is present at about 20% by weight, bacteria or fungi at about 1.47% by weight, FOS, GOS, or other appropriate polysaccharide at about 35% by weight, and buffer is present at about 3% by weight. In an embodiment, lactose is present at about 20% by weight, bacteria or fungi at about 1.47% by weight, FOS, GOS, or other appropriate polysaccharide at about 40% by weight, and buffer is present at about 3% by weight. In an embodiment, lactose is present at about 20% by weight, bacteria or fungi at about 1.47% by weight, FOS, GOS, or other appropriate polysaccharide at about 50% by weight, and buffer is present at about 3% by weight. In an embodiment, lactose is present at about 20% by weight, bacteria or fungi at about 1.47% by weight, FOS, GOS, or other appropriate polysaccharide at about 60% by weight, and buffer is present at about 3% by weight. In an embodiment, lactose is present at about 20% by weight, bacteria or fungi at about 1.47% by weight, FOS, GOS, or other appropriate polysaccharide at about 70% by weight, and buffer is present at about 3% by weight. In an embodiment, lactose is present at about 5% by weight, bacteria or fungi at about 1.47% by weight, FOS, GOS, or other appropriate polysaccharide at about 90% by weight, and buffer is present at about 3% by weight. In an embodiment, lactose is present at about 3% by weight, bacteria or fungi at about 1.47% by weight, FOS, GOS, or other appropriate polysaccharide at about 92% by weight, and buffer is present at about 3% by weight. In an embodiment, lactose is present at about 2% by weight, bacteria or fungi at about 1.47% by weight, FOS, GOS, or other appropriate polysaccharide at about 93% by weight, and buffer is present at about 3% by weight. In an embodiment, lactose is present at about 1% by weight, bacteria or fungi at about 1.47% by weight, FOS, GOS, or other appropriate polysaccharide at about 94% by weight, and buffer is present at about 3% by weight. In an embodiment, lactose is present at about 0.5% by weight, bacteria or fungi at about 1.47% by weight, FOS, GOS, or other appropriate polysaccharide at about 95% by weight, and buffer is present at about 3% by weight. If the bacteria, fungi, FOS, GOS, or other, buffer and lactose do not make up 100% of the composition by weight, the remaining ingredients can be any suitable ingredients intended for consumption by a subject, e.g., human, including, but not limited to, ingredients intended to inhibit clumping and increase pourability, such as silicone dioxide and microcrystalline cellulose, or similar ingredients as are well-additional in the art. Remaining ingredients can also include ingredients to improve handling, preservatives, antioxidants, flavorings and the like.

### I. Additional Ingredients

Additional ingredients include ingredients to improve handling, preservatives, antioxidants, flavorings and the like. For example, in an embodiment, a prebiotic composition in powdered form can include flavorings such that when mixed in a liquid (e.g., water), the powder can flavor the liquid with various flavors such as grape, strawberry, lime, lemon, chocolate, and the like. In an embodiment, the compositions include microcrystalline cellulose or silicone dioxide. Preservatives can include, for example, benzoic acid, alcohols, for example, ethyl alcohol, and hydroxybenzoates. Antioxidants can include, for example, butylated hydroxyanisole (BHA), butylated hydroxytoluene (BHT), tocopherols (e.g., Vitamin E), and ascorbic acid (Vitamin C).

### Dosage Forms

### A. General

Compositions described herein include any suitable form, including liquid or powder. Powdered compositions can be as pure powder, or can be in the form of capsules, tablets, or the like. Powder can be packaged in bulk (e.g., in a container containing sufficient prebiotic or other substances for a subject to follow for an entire course of treatment with increasing doses of prebiotic, or a portion of a course of treatment), or as individual packets (e.g., packets containing a single dose of prebiotic plus other components, or packets containing the dose of prebiotic and other components needed for a particular day of a prebiotic treatment regimen). If packaged in bulk, the powder can be in any suitable container, such as a packet, sachet, canister, ampoule, ramekin, or bottle. The container can also include one or more scoops or similar serving devices of a size or sizes appropriate to measure and serve one or more doses of prebiotic and, optionally, other ingredients included in the powder. Liquid compositions contain prebiotic and, optionally, other ingredients, in a suitable liquid, e.g., water or buffer. Liquid compositions can be provided in bulk (e.g., in a container containing sufficient prebiotic or other substances for one subject in need thereof to follow an entire course of treatment with increasing doses of prebiotic, or a portion of a course of treatment), or as individual containers, such as cans, bottles, soft packs, and the like (e.g., containers containing a single dose of prebiotic plus other components in suitable liquid, or containers containing the dose of prebiotic and other components needed for a particular day of a prebiotic treatment regimen). The container can also include one or more measuring cups or similar serving devices of a size or sizes appropriate to measure and serve one or more doses of prebiotic and, optionally, other ingredients included in the liquid.

In an embodiment, compositions described herein comprise one or more excipients. In an embodiment, the one or more excipients comprise one or more anti-adherents, one or more binders, one or more coatings, one or more disintegrants, one or more fillers, one or more flavors, one or more colors, one or more lubricants, one or more glidants, one or more sorbents, one or more preservatives, one or more sweeteners, or a combination thereof. In an embodiment, the antiadherent is magnesium stearate. In an embodiment, the one or more binders are cellulose, microcrystalline cellulose, hydroxypropyl cellulose, xylitol, sorbitol, maltitol, gelatin, polyvinylpyrrolidone, polyethylene glycol, methyl cellulose, hydroxypropyl methylcellulose, or a combination thereof. In an embodiment, the one or more coatings are a hydroxypropyl methylcellulose film, shellac, corn protein zein, gelatin, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, stearic acid, or a combination thereof. In an embodiment, the one or more disintegrants are crosslinked polyvinylpyrrolidone (crospovidone), crosslinked sodium carboxymethyl cellulose (croscarmellose sodium), sodium starch glycolate, or a combination thereof. In an embodiment, the one or more fillers are calcium carbonate, magnesium stearate, dibasic calcium phosphate, cellulose, vegetable oil, vegetable fat, or a combination thereof. In an embodiment, the one or more flavors are mint, cherry, anise, peach, apricot, licorice, raspberry, vanilla, or a combination thereof. In an embodiment, the one or more lubricants are talc, silica, vegetable stearin, magnesium stearate, stearic acid, or a combination thereof. In an embodiment, the one or more glidants are fumed silica, talc, magnesium carbonate, or a combination thereof. In an embodiment, the one or more sorbents are fatty acids, waxes, shellac, plastics, plant fibers, or a combination thereof. In an embodiment, the one or more preservatives are vitamin A, vitamin E, vitamin C, retinyl palmitate, selenium, cysteine, methionine, citric acid, sodium citrate, methyl paraben, propyl paraben, or a combination thereof. In an embodiment, the one or more sweeteners are stevia, sparame, sucralose, neotame, acesulfame potassium, saccharin or a combination thereof.

### B. Oral Dosage Forms and Components

In one aspect provided herein are methods and compositions formulated for oral delivery to a subject in need thereof. In an embodiment a composition is formulated to deliver a composition comprising a prebiotic to a subject in need thereof. In another embodiment, a pharmaceutical composition is formulated to deliver a composition comprising a prebiotic to a subject in need thereof. In another embodiment a composition is formulated to deliver a composition comprising prebiotic and a probiotic microorganism to a subject in need thereof.

### 1. Forms

In an embodiment, a composition is administered in solid, semi-solid, micro-emulsion, gel, or liquid form. Examples of such dosage forms include tablet forms disclosed in U.S. Pat. Nos. 3,048,526, 3,108,046, 4,786,505, 4,919,939, and 4,950,484; gel forms disclosed in U.S. Pat. Nos. 4,904,479, 6,482,435, 6,572,871, and 5,013,726; capsule forms disclosed in U.S. Pat. Nos. 4,800,083, 4,532,126, 4,935,243, and 6,258,380; or liquid forms disclosed in U.S. Pat. Nos. 4,625,494, 4,478,822, and 5,610,184; each of which is incorporated herein by reference in its entirety.

Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients including freeze-dried plant material serving both as prebiotic and as a filler. Compressed tablets can be prepared by compressing in a suitable machine the active ingredient in a free-flowing form such as a powder or granules, optionally mixed with binders (e.g., povidone, gelatin, hydroxypropylmethyl cellulose), inert diluents, preservative, antioxidant, disintegrant (e.g., sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g., colon, lower intestine) other than the stomach. All formulations for oral administration can be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds (prebiotics or probiotics) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil.

Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel, fruit dry extracts, plant extracts, or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydoxybenzoate or sorbic acid, and, if desired, conventional flavoring or coloring agents.

In an embodiment, a provided composition includes a softgel formulation. A softgel can contain a gelatin-based shell that surrounds a liquid fill. The shell can be made of gelatin, plasticiser (e.g., glycerin and/or sorbitol), modifier, water, color, antioxidant, or flavor. The shell can be made with starch or carrageenan. The outer layer can be enteric coated. In an embodiment, a softgel formulation can include a water or oil soluble fill solution, or suspension of a composition, for example, a prebiotic composition, covered by a layer of gelatin.

An enteric coating can control the location of where a prebiotic composition is absorbed in the digestive system. For example, an enteric coating can be designed such that a prebiotic composition does not dissolve in the stomach, but rather, travels to the small intestine, where it dissolves. An enteric coating can be stable at low pH (such as in the stomach) and can dissolve at higher pH (for example, in the small intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade name aquacoat CPD®, Sepifilm™LP, Klucel®Aquacoat®ECD, and Metolose®); polyvinylacetate phthalate (trade name Sureteric®); and methacrylic acid (trade name Eudragit®).

In an embodiment, an enteric coated prebiotic composition is administered to a subject. In another embodiment, an enteric coated probiotic composition is administered to a subject. In another embodiment, an enteric coated probiotic microorganism and prebiotic composition is administered to a subject. In an embodiment, probiotic microorganisms can be administered to a subject using an enteric coating. The stomach has an acidic environment that can kill probiotics. An enteric coating can protect probiotics as they pass through the stomach and small intestine.

Enteric coatings can be used to (1) prevent the gastric juice from reacting with or destroying the active substance, (2) prevent dilution of the active substance before it reaches the intestine, (3) ensure that the active substance is not released until after the preparation has passed the stomach, and (4) prevent live bacteria contained in the preparation from being killed because of the low pH-value in the stomach.

Enteric coatings can also be used for avoiding irritation of or damage to the mucous membrane of the stomach caused by substances contained in the oral preparation, and for counteracting or preventing formation or release of substances having an unpleasant odor or taste in the stomach. Finally, such coatings can be used for preventing nausea or vomiting on intake of oral preparations.

In an embodiment a prebiotic composition is provided as a tablet, capsule, or caplet with an enteric coating. In an embodiment the enteric coating is designed to hold the tablet, capsule, or caplet together when in the stomach. The enteric coating is designed to hold together in acid conditions of the stomach and break down in non-acid conditions and therefore release the drug in the intestines.

Softgel delivery systems can also incorporate phospholipids or polymers or natural gums to entrap a composition, for example, a prebiotic composition, in the gelatin layer with an outer coating to give desired delayed/control release effects, such as an enteric coating.

Formulations of softgel fills can be at pH 2.5-7.5.

A softgel formulation can be sealed tightly in an automatic manner. A softgel formulation can easily be swallowed, allow for product identification using colors and several shapes, allow uniformity, precision and accuracy between dosages, be safe against adulteration, provide good availability and rapid absorption, and offer protection against contamination, light and oxidation. Furthermore, softgel formulations can avoid unpleasant flavors due to content encapsulation.

A composition comprising a softgel formulation can be in any of number of different sizes, including, for example, round, oblong, oval, tube, droplet, or suppositories.

In an embodiment a composition is provided in a dosage form which comprises an effective amount of prebiotic and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In an embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule.

In an embodiment, the dosage form is a gelatin capsule having a size indicated in Table 1.

TABLE 1
Empty Gelatin Capsule Physical
Specifications Allowable For Human Consumption

| Outer | Height or | Actual |
|---|---|---|
| Diameter | Locked | Volume |
| Size (mm) | Length (mm) | (mL) |
| 9.97 | 26.14 | 1.37 |
| 8.53 | 23.30 | 0.95 |
| 7.65 | 21.70 | 0.68 |
| 6.91 | 19.40 | 0.50 |
| 6.35 | 18.00 | 0.37 |
| 5.82 | 15.90 | 0.30 |
| 5.31 | 14.30 | 0.21 |
| 4.91 | 11.10 | 0.13 |

Note:
sizes and volumes are approximate.

In another embodiment a composition comprising a prebiotic is provided in effervescent dosage forms. The compositions can also comprise non-release controlling excipients.

In another embodiment, a composition comprising a prebiotic is provided in a dosage form that has at least one component that can facilitate release of the prebiotic. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment the prebiotic mixture is a plant or plant extract, either in solid or liquid form.

In another embodiment a composition comprising a prebiotic is provided in an enteric coated dosage form. The composition can also comprise non-release controlling excipients.

In another embodiment a composition comprising a prebiotic is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In an embodiment a composition comprising a prebiotic is provided in the form of enteric-coated granules, for oral administration. The compositions can further comprise cellulose, disodium hydrogen phosphate, hydroxypropyl cellulose, hypromellose, lactose, mannitol, and sodium lauryl sulfate.

In another embodiment a composition comprising a prebiotic is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate.

In an embodiment a composition comprising a prebiotic is provided in the form of enteric-coated granules, for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In another embodiment a composition comprising a prebiotic can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The compositions provided herein can be in unit-dosage forms or multiple-dosage forms. Unit-dosage forms, as used herein, refer to physically discrete units suitable for administration to human or non-human animal subject in need thereof and packaged individually. Each unit-dose can contain a predetermined quantity of an active ingredient(s) sufficient to produce the desired therapeutic effect, in association with other pharmaceutical carriers or excipients. Examples of unit-dosage forms include, but are not limited to, ampoules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents. For example, a multiple dosage form can be provided which comprises a first dosage element comprising a composition comprising a prebiotic and a second dosage element comprising lactose or a probiotic, which can be in a modified release form.

In this example a pair of dosage elements can make a single unit dosage. In an embodiment a kit is provided comprising multiple unit dosages, wherein each unit comprises a first dosage element comprising a composition comprising a prebiotic and a second dosage element comprising probiotic, lactose or both, which can be in a modified release form. In another embodiment the kit further comprises a set of instructions.

In an embodiment, compositions can be formulated in various dosage forms for oral administration. The compositions can also be formulated as a modified release dosage form, including immediate-, delayed-, extended-, prolonged-, sustained-, pulsatile-, controlled-, extended, accelerated-, fast-, targeted-, programmed-release, and gastric retention dosage forms. These dosage forms can be prepared according to additional methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which is herein incorporated by reference in its entirety).

In an embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition comprising a prebiotic. Furthermore, dosage forms of the disclosure can comprise acceptable carriers or salts additional in the art, such as those described in the Handbook of Pharmaceutical Excipients, American Pharmaceutical Association (1986), incorporated by reference herein in its entirety.

In an embodiment, an effective amount of a composition comprising a prebiotic is mixed with a pharmaceutical excipient to form a solid preformulation composition comprising a homogeneous mixture of compounds described herein. When referring to these compositions as “homogeneous,” it is meant that the agents are dispersed evenly throughout the composition so that the composition can be subdivided into unit dosage forms such as tablets, caplets, or capsules. This solid preformulation composition can then be subdivided into unit dosage forms of the type described above comprising from, for example, about 1 g to about 20 mg of a prebiotic composition. A prebiotic composition can be formulated, in the case of caplets, capsules or tablets, to be swallowed whole, for example with water.

The compositions described herein can be in liquid form. The liquid formulations can comprise, for example, an agent in water-in-solution and/or suspension form; and a vehicle comprising polyethoxylated castor oil, alcohol, and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents, and any other inactive agents that can be included in pharmaceutical dosage forms for oral administration. Examples of such vehicles and additives can be found in Remington's Pharmaceutical Sciences, 17th edition (1985).

### 2. Manufacturing

The dosage forms described herein can be manufactured using processes that are well additional to those of skill in the art. For example, for the manufacture of tablets, an effective amount of a prebiotic can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers,” can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc, and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

In an embodiment, a softgel formulation is made with a gelatin mass for the outer shell, and a composition including one or more substances, for example prebiotics and/or probiotic microorganisms, for the capsule fill can be prepared. To make the gelatin mass, gelatin powder can be mixed with water and glycerin, heated, and stirred under vacuum. Additives, for example, flavors or colors, can be added to molten gelatin using a turbine mixer and transferred to mobile vessels. The gelatin mass can be kept in a steam-jacketed storage vessel at a constant temperature.

The encapsulation process can begin when the molten gel is pumped to a machine and two thin ribbons of gel are formed on either side of machine. These ribbons can then pass over a series of rollers and over a set of die that determine the size and shapes of capsules. A fill composition, for example a prebiotic and/or probiotic microorganism fill composition, can be fed to a positive displacement pump, which can dose the fill and inject it between two gelatin ribbons prior to sealing them together through the application of heat and pressure. To remove excess water, the capsules can pass through a conveyer into tumble dryers where a portion of the water can be removed. The capsules can then be placed on, for example, trays, which can be stacked and transferred into drying rooms. In the drying rooms, dry air can be forced over capsules to remove any excess moisture.

### 3. Release Formulations

Immediate-release formulations of an effective amount of a prebiotic composition can comprise one or more combinations of excipients that allow for a rapid release of a pharmaceutically active agent (such as from 1 minute to 1 hour after administration). In an embodiment an excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients.

“Controlled-release” formulations (also referred to as sustained release (SR), extended-release (ER, XR, or XL), time-release or timed-release, controlled-release (CR), or continuous-release) refer to the release of a prebiotic composition from a dosage form at a particular desired point in time after the dosage form is administered to a subject. Controlled-release formulations can include one or more excipients, including but not limited to microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, or Avicel PH200. Generally, controlled-release includes sustained but otherwise complete release. A sudden and total release in the large intestine at a desired and appointed time or a release in the intestines such as through the use of an enteric coating are both considered controlled-release. Controlled-release can occur at a predetermined time or in a predetermined place within the digestive tract. It is not meant to include a passive, uncontrolled process as in swallowing a normal tablet. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284 each of which is incorporated herein by reference in its entirety.

In an embodiment a controlled release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled-release dosage form will exceed the amount of time of release of the drug taken as a normal, passive release tablet. Thus, for example, while all of at least one pharmaceutically active agent of an uncoated aspirin tablet should be released within, for example, four hours, a controlled-release dosage form could release a smaller amount of aspirin over a period of six hours, 12 hours, or even longer. Controlled-release in accordance with the compositions and methods described herein generally means that the release occurs for a period of six hours or more, such as 12 hours or more.

In another embodiment a controlled release dosage refers to the release of an agent, from a composition or dosage form in which the agent is released according to a desired profile over an extended period of time. In an embodiment, controlled-release results in dissolution of an agent within 20-720 minutes after entering the stomach. In another embodiment, controlled-release occurs when there is dissolution of an agent within 20-720 minutes after being swallowed. In another embodiment, controlled-release occurs when there is dissolution of an agent within 20-720 minutes after entering the intestine. In another embodiment, controlled-release results in substantially complete dissolution after at least 1 hour following administration. In another embodiment, controlled-release results in substantially complete dissolution after at least 1 hour following oral administration. For example, controlled-release compositions allow delivery of an agent to a subject in need thereof over an extended period of time according to a predetermined profile. Such release rates can provide therapeutically effective levels of agent for an extended period of time and thereby provide a longer period of pharmacologic or diagnostic response as compared with conventional rapid release dosage forms. Such longer periods of response provide for many inherent benefits that are not achieved with immediate-release dosages. When used in connection with the dissolution profiles discussed herein, the term “controlled-release” refers to wherein all or less than all of the total amount of a dosage form, made according to methods and compositions described herein, delivers an active agent over a period of time greater than 1 hour.

When present in a controlled-release oral dosage form, the compositions described herein can be administered at a substantially lower daily dosage level than immediate-release forms.

In an embodiment, the controlled-release layer is capable of releasing about 30 to about 40% of the one or more active agents (e.g., prebiotic and/or probiotic) contained therein in the stomach of a subject in need thereof in about 5 to about 10 minutes following oral administration. In another embodiment, the controlled-release layer is capable of releasing about 90% of the one or more active agents (e.g., prebiotic and/or probiotic) is released in about 40 minutes after oral administration.

In some embodiments, the controlled-release layer comprises one or more excipients, including but not limited to silicified microcrystalline cellulose (e.g., HD90), croscarmellose sodium (AC-Di-Sol), hydroxyl methyl propyl cellulose, magnesium stearate, or stearic acid. In an embodiment, a controlled release formulation weighs between about 100 mg to 3 g.

Pharmaceutical carriers or vehicles suitable for administration of the compounds provided herein include all such carriers additional to those skilled in the art to be suitable for the particular mode of administration. In addition, the compositions can one or more components that do not impair the desired action, or with components that supplement the desired action, or have another action.

In another embodiment, an effective amount of the prebiotic is formulated in an immediate release form. In this embodiment the immediate-release form can be included in an amount that is effective to shorten the time to its maximum concentration in the blood. By way of example, certain immediate-release pharmaceutical preparations are taught in United States Patent Publication US 2005/0147710A1 entitled, “Powder Compaction and Enrobing,” which is incorporated herein in its entirety by reference.

The dosage forms described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In a further aspect the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a prebiotic). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included. In an embodiment citric acid and sodium bicarbonate are used.

In another aspect the dosage form can be in a candy form (e.g., matrix), such as a lollipop or lozenge. In an embodiment an effective amount of a prebiotic is dispersed within a candy matrix. In an embodiment the candy matrix comprises one or more sugars (such as dextrose or sucrose). In another embodiment the candy matrix is a sugar-free matrix. The choice of a particular candy matrix is subject to wide variation. Conventional sweeteners such as sucrose can be utilized, or sugar alcohols suitable for use with diabetic patients, such as sorbitol or mannitol can be employed. Other sweeteners, such as the aspartames, can also be easily incorporated into a composition in accordance with compositions described herein. The candy base can be very soft and fast dissolving, or can be hard and slower dissolving. Various forms will have advantages in different situations.

A candy mass composition comprising an effective amount of the prebiotic can be orally administered to a subject in need thereof so that an effective amount of the prebiotic will be released into the subject's mouth as the candy mass dissolves and is swallowed. A subject in need thereof includes a human adult or child.

In an embodiment a candy mass is prepared that comprises one or more layers which can comprise different amounts or rates of dissolution of the prebiotic. In an embodiment a multilayer candy mass (such as a lollipop) comprises an outer layer with a concentration of the prebiotic differing from that of one or more inner layers. Such a drug delivery system has a variety of applications.

The choices of matrix and the concentration of the drug in the matrix can be important factors with respect to the rate of drug uptake. A matrix that dissolves quickly can deliver drug into the subject's mouth for absorption more quickly than a matrix that is slow to dissolve. Similarly, a candy matrix that contains the prebiotic in a high concentration can release more of the prebiotic in a given period of time than a candy having a low concentration. In an embodiment a candy matrix such as one disclosed in U.S. Pat. No. 4,671,953 or US Application Publication No. 2004/0213828 (which are herein incorporated by reference in their entirety) is used to deliver the prebiotic.

The dosage forms described herein can also take the form of pharmaceutical particles manufactured by a variety of methods, including but not limited to high-pressure homogenization, wet or dry ball milling, or small particle precipitation (e.g., nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In an embodiment the pharmaceutical particles have a final size of 3-1000 μm, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μm. In another embodiment the pharmaceutical particles have a final size of 10-500 μm. In another embodiment the pharmaceutical particles have a final size of 50-600 μm. In another embodiment the pharmaceutical particles have a final size of 100-800 μm.

In an embodiment an oral dosage form (such as a powder, tablet, or capsule) is provided comprising a prebiotic composition comprising about 0.7 g of FOS, GOS, or other FOS, GOS, or other appropriate polysaccharide, about 0.2 g of lactose, about 0.01 g of glucose, about 0.01 g of galactose, about 0.1-0.2 g of a binder, about 0.1-0.2 g of a dispersant, about 0.1-0.2 g of a solubilizer, wherein the FOS, GOS, or other FOS, GOS, or other appropriate polysaccharide are composed of about 1-25% disaccharides, about 1-25% trisaccharides, about 1-25% tetrasaccharides, and about 1-25% pentasaccharides. The oral dosage form can be in the form of a powder, capsule, or tablet. Suitable amounts of binders, dispersants, and solubilizers are additional in the art for preparation of oral tablets or capsules.

In another embodiment an oral dosage form (such as a powder, tablet or capsule) is provided comprising a prebiotic composition comprising about 1-99.9% by weight of FOS, GOS, or other FOS, GOS, or other appropriate polysaccharide about 0.5-20% by weight of lactose, about 0.1-2% by weight of glucose, about 0.1-2% by weight of galactose, about 0.05-2% by weight of a binder, about 0.05-2% by weight of a dispersant, about 0.05-2% by weight of a solubilizer, wherein the FOS, GOS, or other FOS, GOS, or other appropriate polysaccharide are composed of about 1-25% by weight disaccharides, about 1-25% by weight trisaccharides, about 1-25% by weight tetrasaccharides, and about 1-25% by weight pentasaccharides.

In another embodiment an oral dosage form (such as a powder, tablet, or capsule) is provided comprising a prebiotic composition comprising about 1, 10, 20, 30, 40, 50, 60, 70, 80, 90, 95, 99.5, 100% by weight of FOS, GOS, or other FOS, GOS, or other appropriate polysaccharide about 0, 5, 10, 15, or 20% by weight of lactose, about 0.1, 0.5, 1, or 2% by weight of glucose, about 0.1, 0.5, 1, or 2% by weight of galactose, about 0.05, 0.1, 0.5, 1, or 2% by weight of a binder, about 0.05, 0.1, 0.5, 1, or 2% by weight of a dispersant, about 0.05, 0.1, 0.5, 1, or 2% by weight of a solubilizer, wherein the FOS, GOS, or other FOS, GOS, or other appropriate polysaccharide are composed of about 1, 5, 10, 15, 20, or 25% by weight disaccharides, about 1, 5, 10, 15, 20, or 25% by weight trisaccharides, about 1, 5, 10, 15, 20, or 25% by weight tetrasaccharides, and about 1, 5, 10, 15, 20, or 25% by weight pentasaccharides.

In another embodiment, an oral dosage form is provided comprising a prebiotic composition, wherein the oral dosage form is a syrup. The syrup can comprise about 1%, 5%, 10%, 15%, 20%, 25%, 30%, 35%, 40%, 45%, 50%, 55%, 60%, 65%, 70%, 75%, 80%, or 85% solid. The syrup can comprise about 15%, 20%, 25%, 30%, 35%, 40%, 45%, or 50% liquid, for example, water. The solid can comprise a prebiotic composition. The solid can be, for example, about 1-96%, 10-96%, 20-96%, 30-96%, 40-96%, 50-96%, 60-96%, 70-96%, 80-96%, or 90-96% prebiotic composition. The solid can be, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96% prebiotic composition. In an embodiment a prebiotic composition comprises FOS, GOS, or other FOS, GOS, or other appropriate polysaccharide. In another embodiment a prebiotic composition comprises FOS, GOS, or other FOS, GOS, or other appropriate polysaccharide and another prebiotic. In another embodiment a prebiotic composition comprises FOS, GOS or other and inulin or GOS and FOS.

In an embodiment, the softgel capsule is about 0.25 mL, 0.5 mL, 1.0 mL, 1.25 mL, 1.5 mL, 1.75 mL, or 2.0 mL. In another embodiment, a softgel capsule comprises about 0.1 g to 2.0 g of prebiotic composition. In another embodiment, a softgel capsule comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 g of a prebiotic composition. In an embodiment the prebiotic composition comprises FOS, GOS, or other FOS, GOS, or other appropriate polysaccharide. In another embodiment the prebiotic composition consists essentially of FOS, GOS, or other FOS, GOS, or other appropriate polysaccharide. In another embodiment, a softgel capsule comprises FOS, GOS, or other FOS, GOS, or other appropriate polysaccharide and inulin or FOS.

In another embodiment, the prebiotic composition is delivered in a gelatin capsule containing an amount of FOS, GOS, or other FOS, GOS, or other appropriate polysaccharide within the ranges listed in Table 2. In another embodiment, the number of pills taken per day is within the ranges listed in Table 2.

TABLE 2
Exemplary GOS Dosing
Units in Gel Capsules

| Size | GOS/Pill (g) | # pills per day |
|---|---|---|
| 000 | 1-2 | 1-15 |
| 00 | 0.6-1.5 | 1-25 |
| 0 | 0.4-1.1 | 1-38 |
| 1 | 0.3-0.8 | 1-50 |
| 2 | 0.25-0.6 | 1-60 |
| 3 | 0.2-0.5 | 1-75 |
| 4 | 0.14-0.3 | 1-107 |

In another embodiment, a prebiotic composition is provided that does not contain a preservative. In another embodiment, a prebiotic composition is provided that does not contain an antioxidant. In another embodiment, a prebiotic composition is provided that does not contain a preservative or an antioxidant. In an embodiment a prebiotic composition comprising FOS, GOS, or other FOS, GOS, or other appropriate polysaccharide does not contain a preservative or an antioxidant.

In another embodiment, a prebiotic composition is formulated as a viscous fluid. In another embodiment, a prebiotic composition is formulated such that its water content is low enough that it does not support microbial growth. In an embodiment, this composition is an intermediate-moisture food, with a water activity between 0.6 and 0.85; in another embodiment this composition is a low-moisture food, with a water activity less than 0.6. Low-moisture foods limit microbial growth significantly and can be produced by one of ordinary skill in the art. For example, these products could be produced similarly to a liquid-centered cough drop. In another embodiment, a prebiotic composition is formulated as a viscous fluid without a preservative in a gel capsule. In another embodiment, a prebiotic composition comprising FOS, GOS, or other FOS, GOS, or other appropriate polysaccharide is a viscous fluid. In another embodiment, a prebiotic composition comprises a high percentage of FOS, GOS, or other FOS, GOS, or other appropriate polysaccharide that does not support microbial growth. In another embodiment, the prebiotic composition comprises FOS, GOS, or other FOS, GOS, or other appropriate polysaccharide and inulin or FOS.

In another embodiment, an oral dosage form is provided comprising a prebiotic composition, wherein the oral dosage form is a softgel. In an embodiment the softgel comprises a syrup. In an embodiment the syrup comprises a prebiotic composition. In an embodiment the prebiotic composition comprises FOS, GOS, or other FOS, GOS, or other appropriate polysaccharide. In another embodiment the prebiotic composition comprises more than 80% FOS, GOS, or other FOS, GOS, or other appropriate polysaccharide. In another embodiment the prebiotic composition comprises between 80-99.9% FOS, GOS, or other. In another embodiment the prebiotic composition comprises more than 80% FOS, GOS, or other FOS, GOS, or other appropriate polysaccharide. In another embodiment the prebiotic composition comprises about 80, 81, 82, 83, 84, 85, 86, 87, 88, 89, 90, 91, 92, 93, 94, 95, 96, 97, 98, 99, or 99.9% FOS, GOS, or other FOS, GOS, or other appropriate polysaccharide.

In an embodiment a FOS, GOS, or other FOS, GOS, or other appropriate polysaccharide composition is formulated for delivery in a soft gel capsule. In an embodiment a FOS, GOS, or other FOS, GOS, or other appropriate polysaccharide composition formulated for delivery in a soft gel capsule is a high percentage FOS, GOS, or other FOS, GOS, or other appropriate polysaccharide composition, such as a 90-100% FOS, GOS, or other FOS, GOS, or other appropriate polysaccharide composition (e.g., 90, 91, 92, 93, 94, 95, 96, 97, 98, 99 or 100% FOS, GOS, or other FOS, GOS, or other appropriate polysaccharide composition by weight). In another embodiment a FOS, GOS, or other FOS, GOS, or other appropriate polysaccharide composition formulated for delivery in a soft gel capsule comprises about 95% FOS, GOS, or other FOS, GOS, or other appropriate polysaccharide. In another embodiment a FOS, GOS, or other FOS, GOS, or other appropriate polysaccharide composition formulated for delivery in a soft gel capsule comprises about 96% FOS, GOS, or other FOS, GOS, or other appropriate polysaccharide. In another embodiment, the FOS, GOS, or other FOS, GOS, or other appropriate polysaccharide composition is formulated such that its water content is low enough that it does not support microbial growth. In another embodiment, the FOS, GOS, or other FOS, GOS, or other appropriate polysaccharide composition is formulated as a viscous fluid without a preservative in a gel capsule. In another embodiment, the FOS, GOS, or other FOS, GOS, or other appropriate polysaccharide composition is formulated as a viscous fluid without an antioxidant in a gel capsule. In another embodiment the soft gel capsule comprises about 0.1-2 g of a FOS, GOS, or other FOS, GOS, or other appropriate polysaccharide composition.

In another embodiment a prebiotic composition can be formulated as described, in U.S. Pat. No. 6,750,331, which is herein incorporated by reference in its entirety. A prebiotic composition can be formulated to comprise an oligosaccharide, a foaming component, a water-insoluble dietary fiber (e.g., cellulose or lignin), or a neutralizing component. In an embodiment a prebiotic composition can be in the form of a chewable tablet.

In an embodiment a foaming component can be at least one member selected from the group consisting of sodium hydrogencarbonate, sodium carbonate, and calcium carbonate. In an embodiment a neutralizing component can be at least one member selected from the group consisting of citric acid, L-tartaric acid, fumaric acid, L-ascorbic acid, DL-malic acid, acetic acid, lactic acid, and anhydrous citric acid. In an embodiment a water-insoluble dietary fiber can be at least one member selected from the group consisting of crystalline cellulose, wheat bran, oat bran, cone fiber, soy fiber, and beet fiber. The formulation can contain a sucrose fatty acid ester, powder sugar, fruit juice powder, and/or flavoring material.

Formulations of the provided disclosure can include additive components selected from various additional additives. Such additives include, for example, saccharides (excluding oligosaccharides), sugar alcohols, sweeteners and like excipients, binders, disintegrators, lubricants, thickeners, surfactants, electrolytes, flavorings, coloring agents, pH modifiers, fluidity improvers, and the like. Specific examples of the additives include wheat starch, potato starch, corn starch, dextrin and like starches; sucrose, glucose, fructose, maltose, xylose, lactose and like saccharides (excluding oligosaccharides); sorbitol, mannitol, maltitol, xylitol and like sugar alcohols; calcium phosphate, calcium sulfate and like excipients; starch, saccharides, gelatine, gum arabic, dextrin, methyl cellulose, polyvinylpyrrolidone, polyvinyl alcohol, hydroxypropylcellulose, xanthan gum, pectin, gum tragacanth, casein, alginic acid and like binders and thickeners; leucine, isoleucine, L-valine, sugar esters, hardened oils, stearic acid, magnesium stearate, talc, macrogols and like lubricants; CMC, CMC-Na, CMC-Ca and like disintegrators; polysorbate, lecithin and like surfactants; aspartame, alitame and like dipeptides; silicon dioxide and like fluidity improvers; and stevia, saccharin, and like sweeteners. The amounts of these additives can be properly selected based on their relation to other components and properties of the preparation, production method, etc.

In an embodiment, a FOS, GOS, or other FOS, GOS, or other appropriate polysaccharide composition is a chewable oral dosage formulation. In an embodiment the chewable formulation can comprises between about 1-99.9% FOS, GOS, or other FOS, GOS, or other appropriate polysaccharide. In an embodiment, a FOS, GOS, or other FOS, GOS, or other appropriate polysaccharide composition comprises about 80% FOS, GOS, or other FOS, GOS, or other appropriate polysaccharide about 5% L-ascorbic acid, about 2% anhydrous citric acid, about 3% sodium hydrogencarbonate, about 3% calcium carbonate, about 2% sucrose fatty acid, about 3% fruit juice powder, and about 2% potassium carbonate.

In another embodiment, a FOS, GOS, or other FOS, GOS, or other appropriate polysaccharide composition comprises about 85% FOS, GOS, or other FOS, GOS, or other appropriate polysaccharide, about 5% L-ascorbic acid, about 3% sodium hydrogencarbonate, about 2% sodium carbonate, about 2% sucrose fatty acid ester, about 2% fruit juice powder, and about 1% potassium carbonate.

In another embodiment, a FOS, GOS, or other FOS, GOS, or other appropriate polysaccharide composition comprises about 90% FOS, GOS, or other FOS, GOS, or other appropriate polysaccharide, about 2% L-ascorbic acid, about 1% anhydrous citric acid, about 2% sodium hydrogencarbonate, about 2% sodium carbonate, about 2% sucrose fatty acid ester, and about 1% potassium carbonate.

In another embodiment, a FOS, GOS, or other FOS, GOS, or other appropriate polysaccharide composition comprises about 95% FOS, GOS, or other FOS, GOS, or other appropriate polysaccharide, about 2% L-ascorbic acid, about 1% sodium hydrogencarbonate, and about 2% fruit juice powder. In another embodiment, a FOS, GOS, or other FOS, GOS, or other appropriate polysaccharide composition comprises about 95% FOS, GOS, or other FOS, GOS, or other appropriate polysaccharide and about 5% of L-ascorbic acid, anhydrous citric acid, sodium hydrogencarbonate, calcium carbonate, sucrose fatty acid, fruit juice powder, or potassium carbonate.

In another embodiment, a FOS, GOS, or other FOS, GOS, or other appropriate polysaccharide composition comprises about 95% FOS, GOS, or other FOS, GOS, or other appropriate polysaccharide and about 5% of L-ascorbic acid, anhydrous citric acid, sodium hydrogencarbonate, calcium carbonate, sucrose fatty acid, fruit juice powder, and potassium carbonate.

### Medical Foods

An alternate embodiment of the present disclosure is a formulation as a medical food.

The consuming public has come to understand that foods possess more than basic nutrition (protein, carbohydrate, fat, etc). For example, 95% of consumers agree that “certain foods have health benefits that go beyond basic nutrition and may reduce the risk of disease or other health concerns.” More than 50% of consumers believe that foods can replace the use of drugs. Replacing the use of drugs may have the benefit of reducing the incidence of adverse side effects suffered by patients following a pharmaceutical drug treatment regimen. In fact, medical foods are assumed to be generally safe, as people have historically consumed these foods safely in non-medical contexts.

The compositions of the disclosure may be administered under the supervision of a medical specialist, or may be self-administered. Medical foods could take the form of nutritional shakes or other liquids or meal replacements or delivery by nasogastric enteral feeding tube. Medical foods of the present disclosure could also take the form of a powder capable of being consumed upon addition to suitable food or liquid.

A medical food formulation of the present disclosure could confer benefits of a synthetic composition of probiotics, microbes isolated from nutritionally beneficial plants, prebiotics, and/or other nutritionally beneficial inclusions. The medical food formulation could be consumed to provide a metabolic function different than a foodstuff rather than to obtain nutrition alone. For example, medical foods of the disclosure may also include at least one vitamin or vitamin precursor. Preferred vitamins possess antioxidant properties and include vitamins A, C, and E, and/or their biochemical precursors. Another embodiment of the medical foods of the disclosure also includes at least one trace element, preferably selected from the group consisting of zinc, manganese and selenium. Medical foods of the disclosure also may include at least one additional antioxidant selected from the group consisting of carotenoids, N-acetylcysteine and L-glutamine. It is additional to those of skill in the art how to construct medical foods containing these elements.

Medical foods of the present disclosure would include effective doses of microbes deemed useful for the indication and effective doses of any vitamin, prebiotic, or other beneficial additive not consumed to obtain nutrition but to add a therapeutic benefit mediated by the production of SCFA or other immuno-stimulant molecules when passing through the GI tract.

Typically, the dietary supplements and medical foods of the present disclosure are consumed at least once daily. In certain embodiments, the dietary supplements and medical foods of the disclosure are administered two times per day, optionally once in the morning and once in the afternoon. In certain embodiments, a treatment regime for the dietary supplements or medical foods will continue for four to eight weeks. Depending on such factors as the medical condition being treated and the response of the patient, the treatment regime may be extended. In certain embodiments, a medical food of the present disclosure will be consumed in two servings per day as either a meal replacement or as a snack between meals.

A subject perceived to be at risk from an immune system disorder, metabolic syndrome, obesity, T2D, digestive distress, bone loss, low bone density, chronic inflammation or already suffering from these or associated disorders, can potentially benefit from ingesting the compositions of the disclosure. In certain embodiments, the compositions of the disclosure can effectively ameliorate symptoms and conditions associated with an immune system disorder, T2D, metabolic syndrome, obesity, digestive distress, bone loss, low bone density, or chronic inflammation with natural compounds, which do not show any severe side effects. Furthermore, the present methods are expected to be well-tolerated, for example without causing any discomfort or nausea, and simple to apply.

### Co-Administration

One or more of the microbial entities described herein and/or additional compounds can be co-administered together. The term “co-administration” refers to two or more microbial entities and/or compounds administered in such a way that they exert their pharmacological effects for the same period of time. Such co-administration may be accomplished by simultaneous, contemporaneous or sequential administration of the two or more microbial entities and/or compounds. Compounds can include a therapeutic agent or a drug. A therapeutic agent or drug can be any agent used in the treatment of a human disease. For example in type 2 diabetes or metabolic syndrome, the compound can be an anti-diabetic medication including, but not limited to, metformin, Acarbose, Miglitol, Voglibose, Sitagliptin, Saxagliptin, Liraglutide, Semaglutide, Tirzepatide, Pioglitazone, dipeptidyl peptidase-4 (DPP4)-inhibitors, glucagon-like peptide-1 (GLP-1) receptor analogs, alpha glucosidase inhibitors, thiazolidinedione, and sodium/glucose cotransporter 2 (SGLT2) inhibitors. In another example, the compound can be an anti-osteoporosis medication including, but not limited to, bisphosphonates (e.g., alendronate, risedronate, ibandronate, zolendronate), biologics (e.g., denosumab, romosozumab), selective estrogen receptor mediators (e.g., Raloxifene), or anabolic agents (e.g., teriparatide, abaloparatide).

In some embodiments, the probiotic compositions described herein are co-administered with an effective amount of an additional therapeutic agent or along with an effective drug regimen to improve the efficacy of said therapeutic agent or drug regimen.

In some embodiments, the probiotic compositions described herein are co-administered with an effective amount of an additional therapeutic agent or along with an effective drug regimen to reduce the side effects of said therapeutic agent or drug regimen.

Co-administration encompasses methods given or given in any combination of simultaneously, sequentially, intermittently and/or continuously. In one aspect, the present disclosure provides a method wherein of the two or more microbial entities and/or compounds is administered intermittently. In one aspect, the present disclosure provides a subject with the two or more microbial entities and/or compounds at least ten (10) minutes, fifteen (15) minutes, twenty (20) minutes between administrations. Minutes, thirty (30) minutes, forty (40) minutes, sixty (60) minutes, two (2) hours, three (3) hours, four (4) hours, six (6) hours, eight (8) hours, Ten (10) hours, twelve (12) hours, fourteen (14) hours, eighteen (18) hours, twenty-four (24) hours, thirty-six (36) hours, forty-eight (48) hours, three (3) days, Four (4), five (5), six (6), seven (7), eight (8), nine (9), ten (10), ten (11), twelve (12) days, thirteen (13) days, fourteen (14) days, three (3) weeks, or four (4) weeks of delayed administration. In such embodiments, delayed administration is continued wherein the two or more microbial entities and/or compounds is continued for a given period of time from about ten (10) minutes to about three hundred and sixty-five (365) days. Followed by a pattern that is not administered for a given period of time from about ten (10) minutes to about thirty (30) days. In one aspect, the present disclosure provides a method wherein of the two or more microbial entities and/or compounds is administered intermittently while the remainder is continuously given. The two or more microbial entities and/or compounds can be administered at different intervals.

In one aspect, the present disclosure provides a method of the two or more microbial entities and/or compounds is administered sequentially. In one aspect, the present disclosure provides a method of simultaneously administering of the two or more microbial entities and/or compounds.

In some embodiments, the present disclosure provides a method of administering the two or more microbial entities and/or compounds in one formulation. In some embodiments, the present disclosure provides a method of administering a combination in two (2) compositions, such as wherein the two or more microbial entities are separate from the formulation of a compound (e.g., a drug or therapeutic agent), or wherein the two or more microbial entities are in separate formulations (e.g., a probiotic microorganism, such as a GRAS probiotic microorganism, is separate from the one or more microbial entities presented in Tables 4-8).

The two or more microbial entities and/or compounds can be co-administered via different routes, e.g., a probiotic composition can be administered orally and a drug or therapeutic agent can be administered intravenously, subcutaneously, or any other route appropriate for administration of the drug or therapeutic agent, as will be appreciated by one skilled in the art.

### Methods for Improving Immune Health

In certain aspects, described herein are methods of improving immune health in a subject, the method comprising administering to the subject an effective amount of the pharmaceutical composition, medical food, dietary supplement or solid food stuff described herein. In certain embodiments, the method modulates the level and/or activity of an inflammatory cytokine in a subject. In certain embodiments, the modulating the level and or activity of an inflammatory cytokine, comprises reducing the level and/or activity of at least one inflammatory cytokine selected from the group consisting of IFNγ, IL-12, TNF-α, IL-17, IL-6, IL-1β, IL-10, and combinations thereof. In certain embodiments, the level and/or activity of the at least one inflammatory cytokine is reduced in the serum or select tissue of subject after administration of the pharmaceutical composition, medical food, dietary supplement or solid food stuff compared to a level and/or activity of the at least one inflammatory cytokine prior to administration of the pharmaceutical composition, medical food, dietary supplement or solid food stuff. In certain embodiments, the level and/or activity of the at least one inflammatory cytokine in the serum or select tissue of a human subject after the administration of the effective amount of the pharmaceutical composition, medical food, dietary supplement or solid food stuff. In certain embodiments, the method causes an anti-inflammatory effect in the subject. In certain embodiments, the anti-inflammatory effect is caused by the production of at least one anti-inflammatory metabolite by either the first microbial entity, the second microbial entity or both the first and the second microbial entities. In certain embodiments, the method prevents, reduces the severity of, and/or enables the dietary management of an immune system disorder. In certain embodiments, the immune system disorder is selected from the group consisting of allergic rhinitis, allergic conjunctivitis, allergic bronchial asthma, atopic eczema, ulcerative colitis, Crohn's disease, celiac disease, multiple sclerosis, anaphylaxis, insect sting, drug allergy, food allergy, asthma, eczema, a disorder or condition associated with a pathological Th17 activity, and combinations thereof.

In certain embodiments, the first microbial entity and the second microbial entity synergize to produce an anti-inflammatory effect in a mammalian host. In certain embodiments, the anti-inflammatory effect in a mammalian host is caused by the production at least one anti-inflammatory metabolite by either the first microbial entity, the second microbial entity or both the first and the second microbial entities. In certain embodiments, administering an effective dose of the pharmaceutical composition to a human subject reduces the level and/or activity of at least one inflammatory cytokine selected from the group consisting of IFNγ, IL-12, TNF-α, IL-17, IL-6, IL-1β, IL-10, and combinations thereof relative to a level and/or activity of the inflammatory cytokine in the serum of the human subject; or a tissue of the subject, prior to administering the pharmaceutical composition to the subject.

### EXAMPLES

Below are examples of specific embodiments for carrying out the present disclosure. The examples are offered for illustrative purposes only and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Microbe Isolation, Bacteria and Fungi

Plant-based and fermented foods are rich sources of diverse microbes. A microbial library was developed that contains microbes from these sources as they represent an untapped potential source of novel beneficial microbes. Vegetables typically eaten raw and fermented foods were selected for isolation of microbes of interest. The materials were sourced at the point of distribution in supermarkets selling both conventional and organic farmed vegetables, either washed and ready to eat or without washing. The samples were divided into 50 g portions, thoroughly rinsed with tap water and blended for 30 seconds on high speed. Large particulate matter in the mixtures was then removed with the use of a coarse and then a fine sieve followed by filtration through a 40 μm sieve. The sieved samples from each food source were stored with a cryoprotectant, for example 10% DMSO.

For DNA extraction, the cell suspension containing the plant microbiota, chloroplasts and plant cell debris was centrifuged at slow speed for removing plant material and the resulting supernatant was centrifuged at high speed to pellet microbial cells. The pellet was resuspended in a buffer containing a proprietary plant cell lysis buffer consisting of chelating agents such as EDTA or Versetene EDTA-based chelating agents to remove divalent ions and a suitable non-ionic detergent such as Tween-20, Tween 80, Triton X, and then washed with PBS. DNA was extracted. DNA quality and concentration were measured. DNA libraries were built, and DNA sequencing was performed. Raw paired-end reads were processed for quality control. Taxonomic annotation at the species level of the microbial community for each sample was metagenome using k-mer analysis with kraken2. Plant samples were also inoculated into media that would facilitate the growth of certain types of organisms to generate Solarea Bio enrichments (SBEs). As examples, cultivation with plant filtrates or acetate enriched broth can enrich for microbes capable of growth on plant substrates or low pH-tolerant microbes.

The sieved samples were also diluted and plated onto media that is non-selective, such as tryptic soy agar, or plated on media that is selective for a given microbial type. For example, fungi can be isolated from a sample by plating on a medium such as potato dextrose agar with added chlorotetracycline to prevent bacterial growth. Likewise, bacteria can be isolated away from yeast by added selective agents such as cycloheximide to the medium. Single colonies were then selected and purified by sequential streak isolations or single cell sorting by FACS. These isolates were then assigned a preliminary identification by 16S rDNA or ITS sequencing for bacteria or fungi respectively, before in depth sequencing analysis.

### Example 2: Genome Sequencing

The highest throughput method of determining microbial functional potential begins with bioinformatic analyses. Through sequencing of isolated microbial candidates, it is possible to identify microbes with potentially beneficial phenotypes.

Whole-genome sequencing: whole-genome sequencing was performed. Microbes grown in pure culture were centrifuged at 4000×rpm for 10 min to remove supernatant. Genomic DNA was isolated from microbial pellets via column-based commercial genomic isolation kits. DNA libraries were built. DNA sequencing was performed using either Illumina or Oxford Nanopore sequencing platforms. Raw reads were processed for quality control, and quality-filtered reads were de novo assembled. Nanopore raw sequencing data was converted into a nucleic acid sequence through the “guppy_basecaller” command line software. Library barcodes were removed, and individual reads were separated by source through the “porechop” demultiplexing tool. Following demultiplexing, assembly of contigs was preformed through the “flye” assembly tool.

### Example 3: Annotation of Genomes

Once a microbial genome has been sequenced it is possible to determine its capacity to produce potentially therapeutic metabolites and compounds. Genome annotation was performed to determine the microbe's taxonomy and gene content. To determine the genes, present within each individual genome, the command line software tool, prokka, was used. The assembled contig information derived from genomic sequencing is input into prokka, which initially identifies the locations of all protein coding sequences, after which coding sequences are annotated as specific genes based on a database of all non-fragment Uniprot entries that have transcript evidence (Apeweiler et al., 2004).

To identify specific genes-of-interest that may not be annotated due to low homology, the BLAST+ command line application was used. A genes-of-interest database was constructed, which contains orthologous genes-of-interest from different species. A non-comprehensive list of genes within this database is included in SEQ ID NO: 1557-1727. Genes-of-interest have included the gene pathways involved in short chain fatty acid (propionate and butyrate) biogenesis, indole (indole-3-acetic acid and indole propionic acid), Gamma-aminobutyric acid (GABA), surfactants (surfactin, nisin, fengycin, and iturin), dopamine, secondary bile acids, exopolysaccharide proteins (EPS), and omega 3 fatty acids biosynthesis.

### Example 4. Gut Simulation Experiments

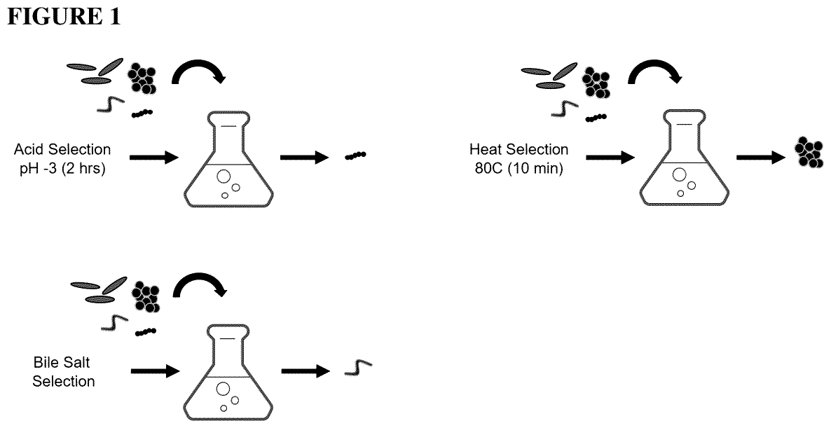

An in vitro system that mimics various sections of the gastrointestinal tract was used to identify probiotic strains of interest. Isolates of interest were incubated in the presence of conditions that mimic particular stresses in the gastro-intestinal tract (such as low pH or bile salts), heat shock, or metformin. After incubation, surviving populations were recovered. Representative isolates identified by this methodology are shown in Table 3 (strains DPT-DP102, corresponding to 16S/ITS SEQ ID NOs: 1-102, respectively). The design layout for this system is depicted in FIG. 1.

TABLE 3
Strains resistant to gut stress, listed with heat shock tolerance,
acid shock tolerance, and isolation temperature.

| | | | | Acid | | |
|---|---|---|---|---|---|---|
| Strain | Strain | | | shock | | |
| Number | No. | Heat | Isolation | (pH 3) | | |
| (SBI) | (DP) | shock | temp | 2 hr | Genus | species |
| SBI189 | DP1 | No | 25 | No | Pseudomonas | fluorescens |
| SBI366 | DP10 | Yes | 25 | No | Bacillus | velezensis |
| SBI3108 | DP100 | No | 30 | No | Lactobacillus | plantarum |
| SBI3220 | DP101 | No | 30 | No | Pediococcus | pentosaceus |
| SBI3298 | DP102 | No | 30 | No | Pichia | kudriavzevii |
| SBI3299 | DP11 | No | 25 | No | Pseudomonas | putida |
| SBI3300 | DP12 | No | 25 | Yes | Microbacterium | sp. |
| SBI3301 | DP13 | No | 25 | Yes | Bacillus | mycoides |
| SBI3302 | DP14 | No | 25 | Yes | Arthrobacter | luteolus |
| SBI3303 | DP15 | No | 25 | No | Curtobacterium | sp. |
| SBI3304 | DP16 | No | 25 | No | Cryptococcus | laurentii |
| SBI3306 | DP17 | No | 25 | No | Rahnella | aquatilis |
| SBI3307 | DP18 | No | 25 | No | Pseudomonas | sp. |
| SBI4260 | DP19 | No | 25 | No | Curtobacterium | pusillum |
| SBI4261 | DP2 | No | 37 | No | Hanseniaspora | occidentalis |
| SBI4263 | DP20 | No | 25 | No | Stenotrophomonas | rhizophila |
| SBI3353 | DP21 | No | 25 | No | Candida | santamariae |
| SBI3316 | DP22 | No | 25 | No | Rahnella | sp. |
| SBI3317 | DP23 | No | 25 | No | Erwinia | billingiae |
| SBI3327 | DP24 | No | 25 | No | Filobasidium | globisporum |
| SBI3329 | DP25 | No | 25 | No | Penicillium | solitum |
| SBI3330 | DP26 | No | 25 | No | Methylobacterium | sp. |
| SBI3332 | DP27 | No | 25 | No | Sphingomonas | sp. |
| SBI3355 | DP28 | No | 25 | Yes | Aureobasidium | pullulans |
| SBI3359 | DP29 | No | 25 | Yes | Pseudoclavibacter | helvolus |
| SBI534 | DP3 | No | 25 | No | Leuconostoc | mesenteroides |
| SBI3405 | DP30 | No | 25 | Yes | Microbacterium | testaceum |
| SBI3406 | DP31 | No | 25 | Yes | Sporisorium | reilianum |
| SBI3438 | DP32 | No | 25 | No | Hafnia | paralvei |
| SBI3440 | DP33 | No | 25 | No | Erwinia | persicinus |
| SBI3473 | DP34 | No | 25 | Yes | Plantibacter | flavus |
| SBI3477 | DP35 | No | 25 | Yes | Pantoea | ananatis |
| SBI3478 | DP36 | No | 25 | Yes | Pantoea | vagans |
| SBI3497 | DP37 | No | 25 | No | Pseudomonas | rhodesiae |
| SBI3498 | DP38 | No | 25 | No | Rhodococcus | sp. |
| SBI3499 | DP39 | No | 25 | No | Agrobacterium | tumefaciens |
| SBI289 | DP4 | No | 25 | No | Aureobasidium | pullulans |
| SBI3528 | DP40 | No | 37 | No | Pantoea | sp. |
| SBI3529 | DP41 | Yes | 37 | No | Corynebacterium | mucifaciens |
| SBI3530 | DP42 | No | 37 | No | Pseudomonas | lundensis |
| SBI3534 | DP43 | No | 25 | No | Janthinobacterium | sp. |
| SBI3535 | DP44 | No | 25 | No | Herbaspirillum | sp. |
| SBI3540 | DP45 | No | 25 | No | Sanguibacter | keddieii |
| SBI3553 | DP46 | No | 25 | Yes | Pantoea | agglomerans |
| SBI3555 | DP47 | No | 25 | Yes | Cronobacter | dublinensis |
| SBI3606 | DP48 | Yes | 25 | No | Bacillus | paralicheniformis |
| SBI3609 | DP49 | Yes | 25 | No | Bacillus | gibsonii |
| SBI319 | DP5 | No | 37 | No | Debaromyces | hansenii |
| SBI3639 | DP50 | No | 25 | No | Enterobacter | sp. |
| SBI3640 | DP51 | No | 25 | No | Klebsiella | aerogenes |
| SBI3641 | DP52 | No | 25 | No | Arthrobacter | sp. |
| SBI3643 | DP53 | No | 25 | No | Pseudomonas | fragi |
| SBI3659 | DP54 | No | 25 | No | Methylobacterium | adhaesivum |
| SBI3670 | DP55 | Yes | 25 | No | Bacillus | megaterium |
| SBI3672 | DP56 | Yes | 25 | No | Paenibacillus | lautus |
| SBI3758 | DP57 | Yes | 25 | No | Bacillus | mycoides |
| SBI3770 | DP58 | No | 25 | No | Janthinobacterium | svalbardensis |
| SBI3772 | DP59 | No | 25 | No | Kosakonia | cowanii |
| SBI548 | DP6 | Yes | 25 | No | Bacillus | cereus |
| SBI3819 | DP60 | Yes | 25 | No | Bacillus | simplex |
| SBI4018 | DP61 | No | 25 | No | Lelliottia | sp. |
| SBI4020 | DP62 | No | 25 | No | Erwinia | sp. |
| SBI4059 | DP63 | No | 25 | Yes | Pseudomonas | azotoformans |
| SBI108 | DP64 | No | 25 | No | Hanseniaspora | uvarum |
| SBI182 | DP65 | No | 25 | No | Bacillus | sp. |
| SBI272 | DP66 | No | 25 | No | Hanseniaspora | occidentalis |
| SBI545 | DP67 | Yes | 25 | No | Bacillus | sp. |
| SBI550 | DP68 | Yes | 25 | No | Bacillus | atrophaeus |
| SBI552 | DP69 | Yes | 25 | No | Bacillus | sp. |
| SBI817 | DP7 | No | 25 | No | Pichia | fermentans |
| SBI564 | DP70 | No | 25 | No | Bacillus | tequilensis |
| SBI1194 | DP71 | No | 25 | No | Rhodosporidium | babjevae |
| SBI1838 | DP72 | Yes | 25 | No | Bacillus | sp. |
| SBI2802 | DP73 | Yes | 37 | No | Bacillus | amyloliquefaciens |
| SBI3025 | DP74 | Yes | 25 | No | Bacillus | coagulans |
| SBI3106 | DP75 | No | 37 | No | Bacillus | valezensis |
| SBI3482 | DP76 | No | 25 | No | Ochrobactrum | sp. |
| SBI3608 | DP77 | Yes | 25 | No | Bacillus | megaterium |
| SBI3625 | DP78 | No | 25 | No | Erwinia | rhapontici |
| SBI3643 | DP79 | No | 25 | No | Pseudomonas | fragi |
| SBI1485 | DP8 | No | 25 | No | Hanseniaspora | opuntiae |
| SBI3659 | DP80 | No | 25 | No | Methylobacterium | adhaesivum |
| SBI3805 | DP81 | Yes | 37 | No | Bacillus | sp. |
| SBI3882 | DP82 | Yes | 37 | No | Bacillus | velezensis |
| SBI3886 | DP83 | Yes | 37 | No | Bacillus | amyloliquefaciens |
| SBI3916 | DP84 | No | 25 | No | Microbacterium | sp. |
| SBI4245 | DP85 | No | 30 | No | Enterococcus | faecium |
| SBI4246 | DP86 | No | 30 | No | Bacillus | velezensis |
| SBI4247 | DP87 | No | 30 | No | Lactobacillus | plantarum |
| SBI4248 | DP88 | No | 30 | No | Bacillus | velezensis |
| SBI4249 | DP89 | No | 30 | No | Bacillus | subtilis |
| SBI2335 | DP9 | No | 25 | No | Pediococcus | pentosaceus |
| SBI4250 | DP90 | No | 30 | No | Lactobacillus | plantarum |
| SBI4251 | DP91 | No | 30 | No | Bacillus | sp. |
| SBI4252 | DP92 | No | 30 | No | Bacillus | tequilensis |
| SBI4255 | DP93 | No | 30 | No | Leuconostoc | mesenteroides |
| SBI4254 | DP94 | No | 30 | No | Lactobacillus | brevis |
| SBI4253 | DP95 | No | 30 | No | Lactobacillus | paracasei |
| SBI4256 | DP96 | No | 30 | No | Lactobacillus | casei |
| SBI4257 | DP97 | No | 30 | No | Lactococcus | garvieae |
| SBI4258 | DP98 | No | 30 | No | Lactococcus | garvieae |
| SBI4259 | DP99 | No | 30 | No | Weissella | cibaria |

### Example 5: Identification of Enzymes Involved in Production of Immunomodulatory Compounds

Microbes including bacteria and fungi are known to produce compounds with immunomodulatory and anti-inflammatory properties including but not limited to short chain fatty acids (SCFA), indoles and indole derivatives, anti-microbial compounds, neurotransmitters such as GABA, serotonin, and dopamine, extracellular polymeric substances (EPS), biosurfactants, secondary bile acids, and polyunsaturated fatty acids. To screen for these compounds in silico, enzyme commission (EC) numbers and amino acid reference sequences were identified for each potential biosynthetic pathway for the production of compounds of interest (SEQ ID NO: 1557-1727). The genes-of-interest database was blasted against the amino acid sequences from the genomes with a 60% identity and 60% query aligned region threshold to identify potential homologs (SEQ ID NO: 1769-1556).

### Example 6: Measurement of Microbially Produced Compounds

The levels of microbially produced metabolites were examined in vitro. Individual microbes were grown in pure culture, after which the microbially conditioned supernatants were examined for metabolites including short chain fatty acids and indole derivatives.

Indole derivatives: In the intestine, tryptophan (Trp) can be metabolized into indole derivatives by the intestinal microbiota that can act as ligands for the aryl hydrocarbon receptor (AhR) in host cells to impact the immune response (Caffaratti et al., 2021, Postler et al., 2017). Indole derivatives including but not limited to indole, indole acetic acid (IAA), and indole propionic acid (IPA) can modulate the production of IL-22, an important mediator of intestinal homeostasis, as well as suppress the activation of NF-κB while simultaneously increasing the production of or anti-inflammatory cytokines to reduce inflammation in the host (Gao et al., 2018). For example, in vitro studies have demonstrated the ability of indole to reduce TNF-α mediated activation of NF-κB, expression of the proinflammatory cytokine IL-8, and induce the production of the anti-inflammatory cytokine IL-10 in HCT-8 cells (Bansal et al., 2010). Thus, screening for microbes that produce indole metabolites will lead to the discovery of microbes with probiotic potential. To detect the presence of indole derivatives from microbes, conditioned supernatant was examined by Salkowski assay, a standard biochemical test that is commonly used to identify the presence of indole-containing compounds with a functional range of detection between 1-100 μM (Sethi et al., 2021). Results for indole derivative production from a selection of examined organisms and consortia can be found in Table 6.

Short chain fatty acid quantification: Short chain fatty acids (SCFA), including acetate, propionate, and butyrate, are produced as a result of anaerobic bacterial fermentation of dietary fibers within the intestine and especially within the colon (Macfarlane et al., 2003). SCFAs have different modes of action on both local and systemic regulation of the immune system. SCFAs regulate and improve the intestinal barrier function by upregulation of the expression of tight junctions (Caffaratti et al., 2021). SCFAs play an important role in T-cell functioning via regulation of G-protein-coupled receptors (GPCRs) and inhibition of histone deacetylase (HDAC) (Caffaratti et al., 2021). One of the most well described and potent anti-inflammatory properties of SCFAs is their capacity to promote regulatory T cells (Tregs) which suppress the activity of effector T cells (Postler et al., 2017). SCFA also inhibit the production of proinflammatory cytokines, including TNF-α, IL-6, and IL-1β, from intestinal macrophages to reduce local and systemic inflammation (Caffaratti et al., 2021). To measure short chain fatty acid production, microbes were grown in pure culture under aerobic or anaerobic conditions. The microbially conditioned supernatant was then examined by gas chromatography (GC) for the presence of acetate, butyrate, and propionate, as previously described (Scortichini et al., 2020). Results for short chain fatty acid production from a selection of examined organisms and consortia can be found in Tables 7-8.

### Example 7: DMA Formulation and In Vitro Testing of DMA Functionality

Microbes in nature generally interact with multiple other groups and form consortia that work in synergy, exchanging metabolic products and substrates resulting in thermodynamically favorable reactions as compared to the individual metabolism. For example, in the human colon, the process for plant fiber depolymerization, digestion and fermentation into butyrate is achieved by multiple metabolic groups working in concert. This type of synergy is reproduced in the DMA concept where strains are selected to be combined based on their ability to synergize to produce anti-inflammatory compounds when exposed to substrates such as plant fibers, tryptophan, or sucrose.

To experimentally describe the process of DMA validation the following method is applied to find candidates applicable for specific products:

Define a suitable habitat where microbes are with desirable attributes are abundant based on ecological hypotheses. For example, fresh vegetables are known to have anti-inflammatory effects when consumed in a whole-food plant-based diet, and therefore, it is likely they harbor microbes that can colonize the human gut.

Apply a selection filter to isolate and characterize only those microbes capable of a relevant function. For example, SCFA production or indole production. In addition, strains need to be compatible with relevant therapeutic drugs.

Selected strains are then cultivated in vitro and their genomes sequenced at 10× coverage to assemble, annotate and use in predictive computational models to predict microbial functions in silico and then validate experimentally using the phenotypic methods described in Example 6.

Microbes with complementary or predicted synergistic functions are then combined. As a non-limiting example, DMA could be assembled from microbes with complementary functions such as Paraclostridium benzoelyticum that produces abundant SCFAs but does not produce any other anti-inflammatory targets and Exiguobacterium sp., which produces IAA, inhibits pathogens, and produces EPS.

Predicted synergistic combinations are then tested in the laboratory to validate functional synergies. Single strains are grown to produce a biomass and the spent growth media is removed after reaching late log or stationary phase. The washed cells are then combined in Defined Microbial Assemblages with about 2-10 different strains per DMA and incubated using a culture media with prebiotic substances and precursors including but not limited to tryptophan, mono or oligosaccharides, or fruit or vegetable powders that promote anti-inflammatory product formation.

DMAs are analyzed for their ability to produce anti-inflammatory compounds in a synergistic manner (i.e., greater production of anti-inflammatory compounds by the combined assemblage as compared to the amount produced by the individual component strains, using the same initial number of cells in the same medium), by applying the range of assays described in Example 6.

### Example 8: Identification of Functional Additivity and Synergy Between DMA Microbes

This example describes an in vitro analysis of the ability of microbes to enhance the growth or metabolite production of another microbe.

Microbes often exist in communities where competition and/or mutualism are present between two or more organisms. This concept of microbial mutualism can be harnessed for the development of DMAs where two or more microbes work in together to grow and/or produce compounds to modulate host immunity. Thus, DMA strain selection can involve identification of microbes that synergize in growth or production of host-beneficial metabolites such as indole derivatives, acetate, or propionate. To identify growth and functional synergies between probiotics and plant-derived microbes, a set of two-member DMAs consisting of one probiotic species and one plant derived species were designed (Table 4).

Microbes are well-known to enhance the growth of one another, often through metabolic cross-feeding, as is seen in cheese production where Lactobacilli spp. produce lactic acid during milk fermentation that is used by propionic acid bacteria to grow (Chamba and Perreard, 2002).

Combinations consisting of one probiotic species and one additional plant-derived species were evaluated for their ability to grow in a commensal (one microbe's growth is enhanced) or synergistic (enhancement of both microbes' growth) manner in vitro. Briefly, individual microbial strains were grown for 24-48 hours to achieve a high OD in tryptic soy broth (TSB) for bacteria and potato dextrose broth (PDB) for yeast. Cultures were normalized to achieve a uniform density and inoculated into BHI and grown for 48 hours anaerobically. Individual and combination microbes were plated at the beginning and end of the experiment on the appropriate media to enumerate lactic acid bacteria (using MRS medium), Bacillus spp. (using TSA medium with cycloheximide), or yeast (using PDA medium with chlorotetracycline). Commensal interactions were defined as less than 70% change in growth of one organism and a 2-fold or greater enhancement of colony forming units for the other organism. Synergistic interactions were identified when both organisms' growth was enhanced by 2-fold increased colony forming units or more. Table 5 provides examples of commensal and synergistic growth between probiotic species and plant-derived microbial species. For example, note DMA 280, where the first organism's growth increased by a factor of one million and the second organism's growth by tenfold, resulting in a combined growth enhancement of 63,000-fold in the liquid culture, greatly surpassing the simple additive effects of individual growth.

The production of indole derivatives such as IAA is important for microbe-host interactions and instances of synergistic enhancement of IAA production between microbes has been documented (Cheng et al., 2024). This synergy can be harnessed in DMAs to suppress inflammation.

Combinations consisting of one probiotic species and one additional plant-derived species were evaluated for their ability to produce indole in an additive or synergistic manner in vitro. Briefly, individual microbial strains were grown for 24-48 hours to achieve a high OD in tryptic soy broth (TSB) for bacteria and potato dextrose broth (PDB) for yeast. Cultures were normalized to achieve a uniform density and inoculated into MTGE supplemented with 0.1% tryptophan and grown for 48 hours aerobically. Cultures were pelleted by centrifugation and supernatants were removed. Indole derivative production was determined by Salkowski assay. Additive production was defined as DMA indole derivative production equivalent to the sum production by each constituent microbe. Synergistic indole derivative production was defined as production by the DMA that exceeded the sum production of the constituent microbes (Table 6). For several DMAs that were synergistic for indole derivatives, only one organism had measurable indole production, implying that co-cultivation enhanced production by the indole-producing partner.

Synergy in SCFA production is a cornerstone of DMA development. This enhanced SCFA production can have more potent immunomodulatory effects. For example, synergistic acetate production been key in the development of a DMA for bone health (Lawenius et al., 2021).

Combinations consisting of one probiotic species and one additional plant-derived species were evaluated for their ability to produce acetate or propionate in an additive or synergistic manner in vitro. Briefly, the microbially conditioned supernatant from the growth synergy experiments described above were examined by gas chromatography (GC) for the presence of acetate and propionate, as previously described (Scortichini et al., 2020). Additive production was defined as DMA SCFA production equivalent to the sum production by each constituent microbe. Synergistic SCFA production occurred when DMA production exceeded the sum production of the constituent microbes (Tables 7 and 8). For several DMAs that were synergistic for SCFAs only one organism had measurable SCFA production, implying that co-cultivation enhanced production by the SCFA-producing partner.

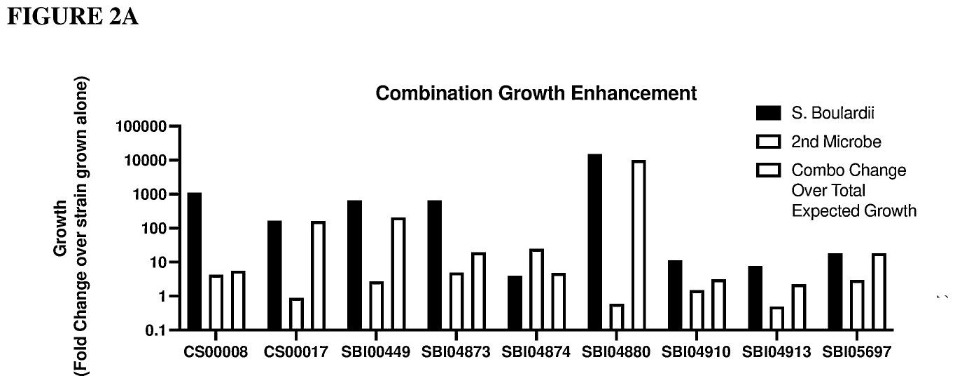

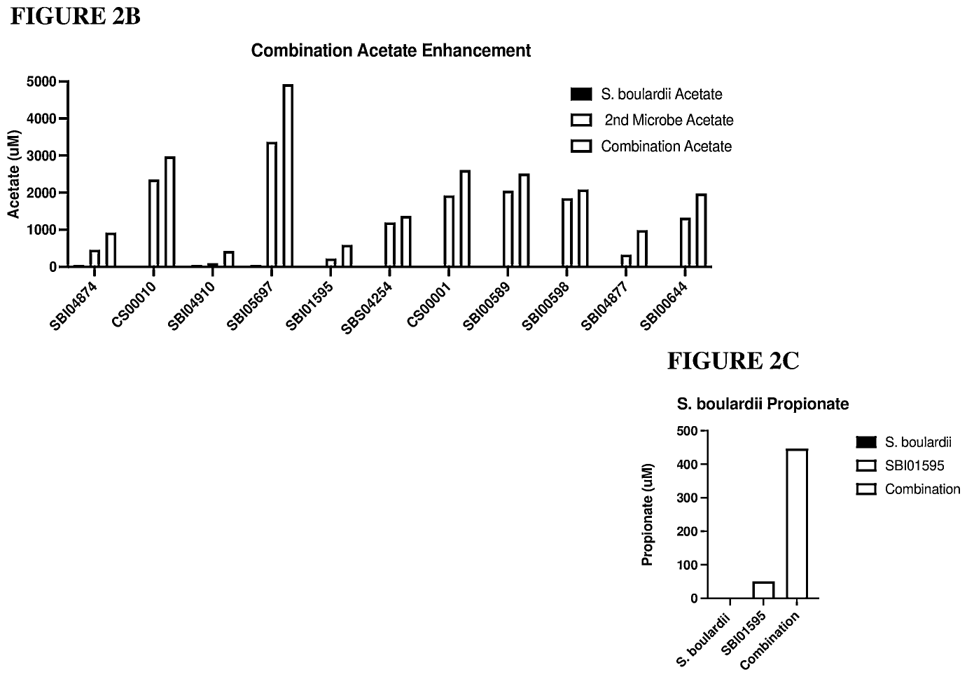

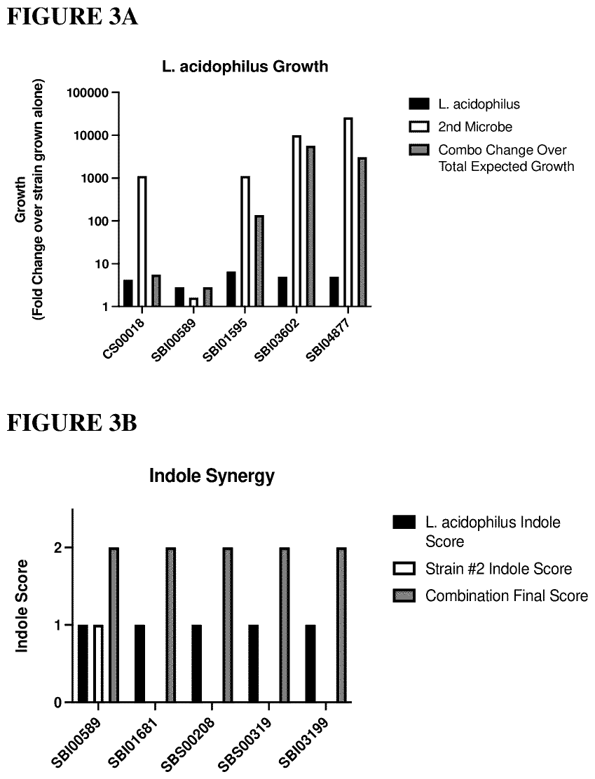

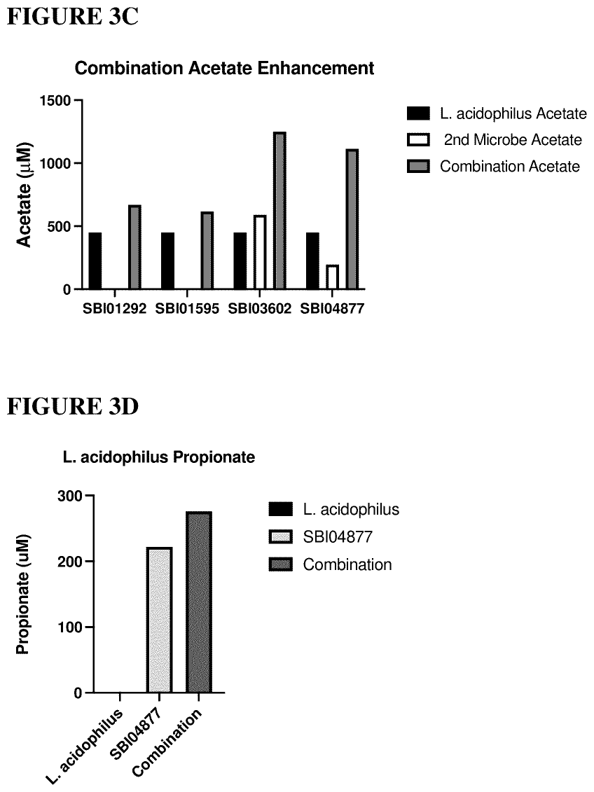

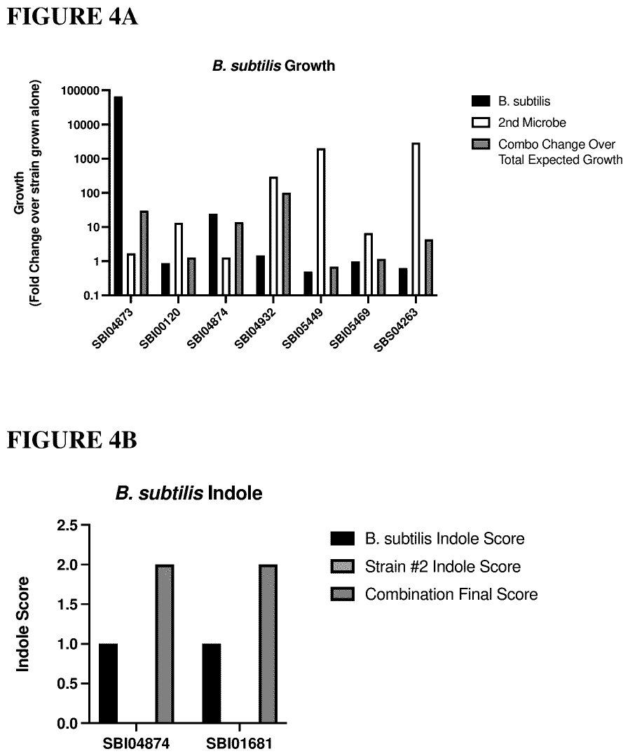

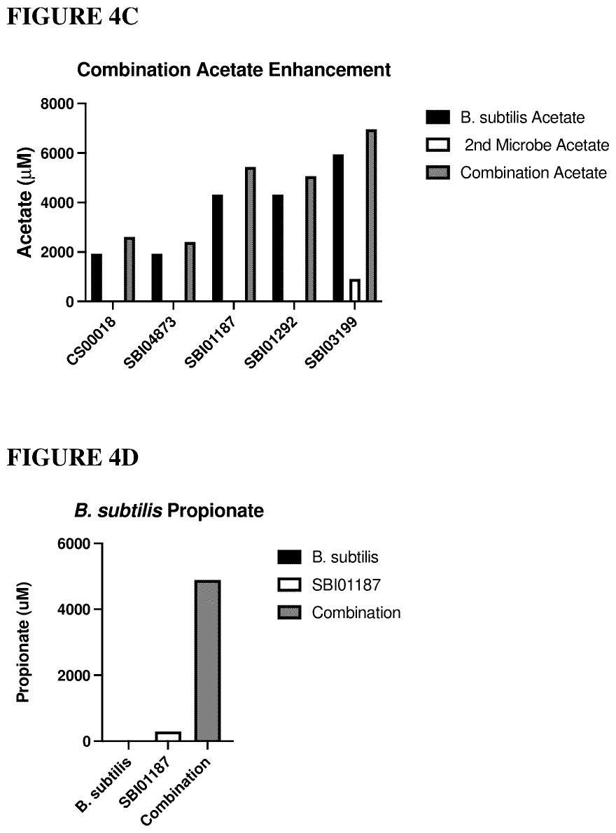

Non-limiting examples of functional synergies demonstrated for microbial growth, acetate production, propionate production, and indole derivative productions are depicted for combinations with Saccharomyces cerevisiae var. boulardii in FIG. 2, for Lactobacillus acidophilus La-14 in FIG. 3, and for Bacillus subtilis DE111 in FIG. 4.

TABLE 4
Strain Composition of DMAs

| DMA | | | | Strain | | | | Strain |
|---|---|---|---|---|---|---|---|---|
| Number | Strain #1 | Genus | Species | ID | Strain #2 | Genus | Species | ID |
| DMA 23 | SBS02335 | Pediococcus | pentosaceus | | SBI00461 | Bacillus | safensis | |
| DMA 24 | SBS02335 | Pediococcus | pentosaceus | | SBI00589 | Bacillus | axarquiensis | |
| DMA 26 | SBS04255 | Leuconostoc | mesenteroides | | SBI00461 | Bacillus | safensis | |
| DMA 27 | SBS04255 | Leuconostoc | mesenteroides | | SBI00589 | Bacillus | axarquiensis | |
| DMA 28 | SBS04255 | Leuconostoc | mesenteroides | | SBI00598 | Bacillus | subtilis | |
| DMA 29 | SBS04255 | Leuconostoc | mesenteroides | | SBI05473 | Saccharomyces | cerevisiae | |
| DMA 30 | SBI04919 | Pediococcus | parvulus | | SBI00461 | Bacillus | safensis | |
| DMA 31 | SBI05399 | Lactiplantibacillus | plantarum | | SBI00461 | Bacillus | safensis | |
| DMA 32 | SBI05399 | Lactiplantibacillus | plantarum | | SBI00598 | Bacillus | subtilis | |
| DMA 33 | SBI05399 | Lactiplantibacillus | plantarum | | SBI05473 | Saccharomyces | cerevisiae | |
| DMA 34 | SBI04256 | Lacticaseibacillus | casei | | SBI00817 | Pichia | fermentans | |
| DMA 37 | SBI04887 | Lacticaseibacillus | casei | | SBI00817 | Pichia | fermentans | |
| DMA 38 | SBI04895 | Lacticaseibacillus | casei | | SBI00817 | Pichia | fermentans | |
| DMA 40 | SBI04887 | Lacticaseibacillus | casei | | SBI01683 | Pichia | fermentans | |
| DMA 43 | SBI04887 | Lacticaseibacillus | casei | | SBS04263 | Pichia | kudriavzevii | |
| DMA 44 | SBI04889 | Lactiplantibacillus | plantarum | | SBS04263 | Pichia | kudriavzevii | |
| DMA 45 | SBI04895 | Lacticaseibacillus | casei | | SBS04263 | Pichia | kudriavzevii | |
| DMA 46 | SBI04256 | Lacticaseibacillus | casei | | SBI00550 | Bacillus | atrophaeus | |
| DMA 48 | SBI04887 | Lacticaseibacillus | casei | | SBI00997 | Bacillus | safensis | |
| DMA 49 | SBI04887 | Lacticaseibacillus | casei | | SBI01819 | Bacillus | pumilus | |
| DMA 50 | SBI04256 | Lacticaseibacillus | casei | | SBI02185 | Bacillus | licheniformis | |
| DMA 51 | SBI04887 | Lacticaseibacillus | casei | | SBI02185 | Bacillus | licheniformis | |
| DMA 52 | SBI04889 | Lactiplantibacillus | plantarum | | SBI02185 | Bacillus | licheniformis | |
| DMA 53 | SBI04887 | Lacticaseibacillus | casei | | SBI04173 | Bacillus | paralicheniformis | |
| DMA 54 | SBI04889 | Lactiplantibacillus | plantarum | | SBI04173 | Bacillus | paralicheniformis | |
| DMA 55 | SBI04895 | Lacticaseibacillus | caset | | SBI04173 | Bacillus | paralicheniformis | |
| DMA 56 | SBI00550 | Bacillus | atrophaeus | | SBI00817 | Pichia | fermentans | |
| DMA 57 | SBI04173 | Bacillus | paralicheniformis | | SBI01187 | Pichia | membranifaciens | |
| DMA 58 | SBI02185 | Bacillus | licheniformis | | SBI01683 | Pichia | fermentans | |
| DMA 59 | SBI04173 | Bacillus | paralicheniformis | | SBI02945 | Meyerozyma | guilliermondii | |
| DMA 61 | SBI02185 | Bacillus | licheniformis | | SBS04263 | Pichia | kudriavzevii | |
| DMA 62 | SBI04173 | Bacillus | paralicheniformis | | SBS04263 | Pichia | kudriavzevii | |
| DMA 64 | SBI01819 | Bacillus | pumilus | | SBI04910 | Pediococcus | ethanolidurans | |
| DMA 65 | SBI02185 | Bacillus | licheniformis | | SBI04910 | Pediococcus | ethanolidurans | |
| DMA 66 | SBI04173 | Bacillus | paralicheniformis | | SBI04910 | Pediococcus | ethanolidurans | |
| DMA 68 | SBI04877 | Bacillus | amyloliquefaciens | | SBI04910 | Pediococcus | ethanolidurans | |
| DMA 69 | SBI00550 | Bacillus | atrophaeus | | SBI04913 | Schleiferilactobacillus | harbinensis | |
| DMA 71 | SBI01819 | Bacillus | pumilus | | SBI04913 | Schleiferilactobacillus | harbinensis | |
| DMA 72 | SBI02185 | Bacillus | licheniformis | | SBI04913 | Schleiferilactobacillus | harbinensis | |
| DMA 73 | SBI04173 | Bacillus | paralicheniformis | | SBI04913 | Schleiferilactobacillus | harbinensis | |
| DMA 75 | SBI04877 | Bacillus | amyloliquefaciens | | SBI04913 | Schleiferilactobacillus | harbinensis | |
| DMA 76 | SBI00997 | Bacillus | safensis | | SBI04916 | Lactococcus | lactis | |
| DMA 77 | SBI03005 | Bacillus | altitudinis | | SBI04918 | Lactococcus | lactis | |
| DMA 78 | SBI00550 | Bacillus | atrophaeus | | SBI05697 | Lactiplantibacillus | plantarum | |
| DMA 79 | SBI00997 | Bacillus | safensis | | SBI05697 | Lactiplantibacillus | plantarum | |
| DMA 80 | SBI01819 | Bacillus | pumilus | | SBI05697 | Lactiplantibacillus | plantarum | |
| DMA 81 | SBI02185 | Bacillus | licheniformis | | SBI05697 | Lactiplantibacillus | plantarum | |
| DMA 82 | SBI04173 | Bacillus | paralicheniformis | | SBI05697 | Lactiplantibacillus | plantarum | |
| DMA 83 | SBI03005 | Bacillus | altitudinis | | SBI05697 | Lactiplantibacillus | plantarum | |
| DMA 84 | SBI04877 | Bacillus | amyloliquefaciens | | SBI05697 | Lactiplantibacillus | plantarum | |
| DMA 85 | SBI04874 | Lentilactobacillus | buchneri | | CS00018 | Saccharomyces | boulardii | |
| DMA 86 | SBI04874 | Lentilactobacillus | buchneri | | SBI05419 | Kazachstania | servazzii | |
| DMA 87 | SBI04874 | Lentilactobacillus | buchneri | | SBI05449 | Kluyveromyces | marxianus | |
| DMA 88 | SBI04874 | Lentilactobacillus | buchneri | | SBI00553 | Bacillus | subtilis | |
| DMA 89 | SBI04874 | Lentilactobacillus | buchneri | | SBI00449 | Exiguobacterium | sp. | |
| DMA 90 | SBI04874 | Lentilactobacillus | buchneri | | SBI00556 | Paenibacillus | sp. | |
| DMA 91 | SBI04874 | Lentilactobacillus | buchneri | | SBI00564 | Bacillus | subtilis | |
| DMA 92 | SBI04873 | Pediococcus | ethanolidurans | | CS00018 | Saccharomyces | boulardii | |
| DMA 93 | SBI04873 | Pediococcus | ethanolidurans | | SBI05449 | Kluyveromyces | marxianus | |
| DMA 94 | SBI04873 | Pediococcus | ethanolidurans | | SBI00553 | Bacillus | subtilis | |
| DMA 97 | SBI04873 | Pediococcus | ethanolidurans | | SBI00564 | Bacillus | subtilis | |
| DMA 98 | CS00010 | Lacticaseibacillus | rhamnosus | Lr-32 | CS00018 | Saccharomyces | boulardii | |
| DMA 99 | CS00010 | Lacticaseibacillus | rhamnosus | Lr-32 | SBI05449 | Kluyveromyces | marxianus | |
| DMA 100 | SBI04902 | Pediococcus | pentosaceus | | SBI00553 | Bacillus | subtilis | |
| DMA 101 | SBI04910 | Pediococcus | ethanolidurans | | CS00018 | Saccharomyces | boulardii | |
| DMA 102 | SBI04910 | Pediococcus | ethanolidurans | | SBI05419 | Kazachstania | servazzii | |
| DMA 103 | SBI04910 | Pediococcus | ethanolidurans | | SBI05449 | Kluyveromyces | marxianus | |
| DMA 104 | SBI04910 | Pediococcus | ethanolidurans | | SBI00553 | Bacillus | subtilis | |
| DMA 107 | SBI04910 | Pediococcus | ethanolidurans | | SBI00564 | Bacillus | subtilis | |
| DMA 108 | SBI04913 | Lactobacillus | harbinensis | | CS00018 | Saccharomyces | boulardii | |
| DMA 109 | SBI04913 | Schleiferilactobacillus | harbinensis | | SBI05449 | Kluyveromyces | marxianus | |
| DMA 110 | SBI04913 | Schleiferilactobacillus | harbinensis | | SBI00553 | Bacillus | subtilis | |
| DMA 114 | SBI05697 | Lactiplantibacillus | plantarum | | CS00018 | Saccharomyces | boulardii | |
| DMA 115 | SBI05697 | Lactiplantibacillus | plantarum | | SBI01679 | Candida | sp. | |
| DMA 116 | SBI05697 | Lactiplantibacillus | plantarum | | SBI05419 | Kazachstania | servazzii | |
| DMA 117 | SBI05697 | Lactiplantibacillus | plantarum | | SBI05449 | Kluyveromyces | marxianus | |
| DMA 118 | SBI05697 | Lactiplantibacillus | plantarum | | SBI05469 | Nakazawaea | ishiwadae | |
| DMA 119 | SBI05697 | Lactiplantibacillus | plantarum | | SBI00553 | Bacillus | subtilis | |
| DMA 120 | SBI05697 | Lactiplantibacillus | plantarum | | SBI00449 | Exiguobacterium | sp. | |
| DMA 121 | SBI05697 | Lactiplantibacillus | plantarum | | SBI00556 | Paenibacillus | sp. | |
| DMA 122 | SBI05697 | Lactiplantibacillus | plantarum | | SBI00564 | Bacillus | subtilis | |
| DMA 123 | SBI05697 | Lactiplantibacillus | plantarum | | SBI00568 | Psychrobacillus | sp. | |
| DMA 124 | SBI00553 | Bacillus | subtilis | | SBI04881 | Lentilactobacillus | buchneri | |
| DMA 125 | SBI00553 | Bacillus | subtilis | | SBI05399 | Lactiplantibacillus | plantarum | |
| DMA 128 | SBI05449 | Kluyveromyces | marxianus | | SBI03599 | Bacillus | endophyticus | |
| DMA 129 | SBI00449 | Exiguobacterium | sp. | | CS00018 | Saccharomyces | boulardii | |
| DMA 130 | SBI00449 | Exiguobacterium | sp. | | SBI05449 | Kluyveromyces | marxianus | |
| DMA 131 | SBI00449 | Exiguobacterium | sp. | | SBS04255 | Leuconostoc | mesenteroides | |
| DMA 132 | SBI00449 | Exiguobacterium | sp. | | SBI04881 | Lentilactobacillus | buchneri | |
| DMA 133 | SBI00449 | Exiguobacterium | sp. | | SBI04919 | Pediococcus | parvulus | |
| DMA 134 | SBI00556 | Paenibacillus | sp. | | CS00018 | Saccharomyces | boulardii | |
| DMA 135 | SBI00556 | Paenibacillus | sp. | | SBI05449 | Kluyveromyces | marxianus | |
| DMA 137 | SBI00556 | Paenibacillus | sp. | | SBI04881 | Lentilactobacillus | buchneri | |
| DMA 138 | SBI00564 | Bacillus | subtilis | | SBI05419 | Kazachstania | servazzii | |
| DMA 139 | SBI00564 | Bacillus | subtilis | | SBI05449 | Kluyveromyces | marxianus | |
| DMA 140 | SBI00564 | Bacillus | subtilis | | SBS04255 | Leuconostoc | mesenteroides | |
| DMA 141 | SBI00564 | Bacillus | subtilis | | SBI04881 | Lentilactobacillus | buchneri | |
| DMA 142 | SBI00564 | Bacillus | subtilis | | SBI04919 | Pediococcus | parvulus | |
| DMA 143 | SBI00564 | Bacillus | subtilis | | SBI04900 | Leuconostoc | pseudomesenteroides | |
| DMA 379 | SBI00564 | Bacillus | subtilis | | SBI05399 | Lactiplantibacillus | plantarum | |
| DMA 145 | SBI00568 | Psychrobacillus | sp. | | SBI05399 | Lactiplantibacillus | plantarum | |
| DMA 146 | SBI01595 | Bacillus | amyloliquefaciens | | CS00018 | Saccharomyces | boulardii | |
| DMA 147 | SBI01595 | Bacillus | amyloliquefaciens | | SBI05419 | Kazachstania | servazzii | |
| DMA 148 | SBI01595 | Bacillus | amyloliquefaciens | | SBI05449 | Kluyveromyces | marxianus | |
| DMA 149 | SBI01595 | Bacillus | amyloliquefaciens | | SBI05469 | Nakazawaea | ishiwadae | |
| DMA 150 | SBI01595 | Bacillus | amyloliquefaciens | | SBS02335 | Pediococcus | pentosaceus | |
| DMA 151 | SBI01595 | Bacillus | amyloliquefaciens | | SBI04881 | Lentilactobacillus | buchneri | |
| DMA 152 | SBI01595 | Bacillus | amyloliquefaciens | | SBI04919 | Pediococcus | parvulus | |
| DMA 153 | SBI01595 | Bacillus | amyloliquefaciens | | SBI04900 | Leuconostoc | pseudomesenteroides | |
| DMA 154 | SBI01595 | Bacillus | amyloliquefaciens | | SBI05399 | Lactiplantibacillus | plantarum | |
| DMA 155 | SBI01679 | Candida | sp. | | SBI00592 | Bacillus | mojavensis | |
| DMA 156 | SBI01683 | Pichia | fermentans | | SBI00592 | Bacillus | mojavensis | |
| DMA 157 | SBS04263 | Pichia | kudriavzevii | | SBI00592 | Bacillus | mojavensis | |
| DMA 158 | SBI01679 | Candida | sp. | | SBI00693 | Bacillus | subtilis | |
| DMA 159 | SBI01683 | Pichia | fermentans | | SBI00693 | Bacillus | subtilis | |
| DMA 160 | SBS04263 | Pichia | kudriavzevii | | SBI00693 | Bacillus | subtilis | |
| DMA 161 | SBI01187 | Pichia | membranifaciens | | SBI00714 | Bacillus | subtilis | |
| DMA 162 | SBI01679 | Candida | sp. | | SBI00714 | Bacillus | subtilis | |
| DMA 163 | SBI01683 | Pichia | fermentans | | SBI00714 | Bacillus | subtilis | |
| DMA 164 | SBI02945 | Meyerozyma | guilliermondii | | SBI00714 | Bacillus | subtilis | |
| DMA 165 | SBS04263 | Pichia | kudriavzevii | | SBI00714 | Bacillus | subtilis | |
| DMA 166 | SBI01187 | Pichia | membranifaciens | | SBI02181 | Bacillus | amyloliquefaciens | |
| DMA 167 | SBS04263 | Pichia | kudriavzevii | | SBI02181 | Bacillus | amyloliquefaciens | |
| DMA 168 | SBI01187 | Pichia | membranifaciens | | SBI02354 | Bacillus | pumilus | |
| DMA 169 | SBI01292 | Talaromyces | atroroseus | | SBI02354 | Bacillus | pumilus | |
| DMA 170 | SBI01679 | Candida | sp. | | SBI02354 | Bacillus | pumilus | |
| DMA 171 | SBI01683 | Pichia | fermentans | | SBI02354 | Bacillus | pumilus | |
| DMA 172 | SBS04263 | Pichia | kudriavzevii | | SBI02354 | Bacillus | pumilus | |
| DMA 178 | SBI03602 | Bacillus | subtilis | | SBI01187 | Pichia | membranifaciens | |
| DMA 179 | SBI01187 | Pichia | membranifaciens | | SBI04006 | Bacillus | subtilis | |
| DMA 180 | SBI01679 | Candida | sp. | | SBI04006 | Bacillus | subtilis | |
| DMA 181 | SBI01187 | Pichia | membranifaciens | | CS00001 | Bacillus | subtilis | DE111 |
| DMA 182 | SBS04263 | Pichia | kudriavzevii | | CS00001 | Bacillus | subtilis | DE111 |
| DMA 183 | SBI01679 | Candida | sp. | | SBS04260 | Lactiplantibacillus | plantarum | |
| DMA 184 | SBI04932 | Galactomyces | geotrichum | | SBI00592 | Bacillus | mojavensis | |
| DMA 185 | SBI05419 | Kazachstania | servazzii | | SBI00592 | Bacillus | mojavensis | |
| DMA 186 | SBI05449 | Kluyveromyces | marxianus | | SBI00592 | Bacillus | mojavensis | |
| DMA 187 | SBI00264 | Clavispora | sp. | | SBI00592 | Bacillus | mojavensis | |
| DMA 188 | SBS00319 | Debaryomyces | hansenii | | SBI00592 | Bacillus | mojavensis | |
| DMA 189 | SBI03199 | Candida | dosseyi | | SBI00592 | Bacillus | mojavensis | |
| DMA 190 | SBI05473 | Saccharomyces | cerevisiae | | SBI00592 | Bacillus | mojavensis | |
| DMA 191 | SBI04932 | Galactomyces | geotrichum | | SBI00693 | Bacillus | subtilis | |
| DMA 192 | SBI05419 | Kazachstania | servazzii | | SBI00693 | Bacillus | subtilis | |
| DMA 193 | SBI05449 | Kluyveromyces | marxianus | | SBI00693 | Bacillus | subtilis | |
| DMA 194 | SBI00264 | Clavispora | sp. | | SBI00693 | Bacillus | subtilis | |
| DMA 195 | SBS04254 | Levilactobacillus | brevis | | SBI00120 | Holtermanniella | takashimae | |
| DMA 196 | SBI04916 | Lactococcus | lactis | | SBI00120 | Holtermanniella | takashimae | |
| DMA 197 | SBS04260 | Lactiplantibacillus | plantarum | | SBI00120 | Holtermanniella | takashimae | |
| DMA 198 | SBI01292 | Talaromyces | atroroseus | | SBI04918 | Lactococcus | lactis | |
| DMA 199 | SBI01292 | Talaromyces | atroroseus | | SBS04260 | Lactiplantibacillus | plantarum | |
| DMA 200 | SBI03599 | Bacillus | endophyticus | | SBI04916 | Lactococcus | lactis | |
| DMA 201 | SBS04260 | Lactiplantibacillus | plantarum | | SBI03599 | Bacillus | endophyticus | |
| DMA 202 | SBS04260 | Lactiplantibacillus | plantarum | | SBI00592 | Bacillus | mojavensis | |
| DMA 203 | SBS04260 | Lactiplantibacillus | plantarum | | SBI00693 | Bacillus | subtilis | |
| DMA 204 | SBS04260 | Lactiplantibacillus | plantarum | | SBI00714 | Bacillus | subtilis | |
| DMA 205 | SBS04260 | Lactiplantibacillus | plantarum | | SBI02181 | Bacillus | amyloliquefaciens | |
| DMA 206 | SBS04260 | Lactiplantibacillus | plantarum | | SBI04006 | Bacillus | subtilis | |
| DMA 207 | SBI00592 | Bacillus | mojavensis | | SBI00120 | Kwoniella | shandongens | |
| DMA 208 | SBI00120 | Kwoniella | shandongens | | SBI00693 | Bacillus | subtilis | |
| DMA 209 | SBI00714 | Bacillus | subtilis | | SBI00120 | Kwoniella | shandongens | |
| DMA 210 | SBI00120 | Kwoniella | shandongens | | SBI02181 | Bacillus | amyloliquefaciens | |
| DMA 211 | SBI00120 | Kwoniella | shandongens | | SBI04006 | Bacillus | subtilis | |
| DMA 214 | SBI01292 | Talaromyces | atroroseus | | SBI00592 | Bacillus | mojavensis | |
| DMA 215 | SBI01292 | Talaromyces | atroroseus | | SBI00693 | Bacillus | subtilis | |
| DMA 216 | SBI01292 | Talaromyces | atroroseus | | SBI00714 | Bacillus | subtilis | |
| DMA 217 | SBI01292 | Talaromyces | atroroseus | | SBI04006 | Bacillus | subtilis | |
| DMA 219 | SBI00693 | Bacillus | subtilis | | SBI04256 | Lacticaseibacillus | casei | |
| DMA 220 | SBI00714 | Bacillus | subtilis | | SBI04256 | Lacticaseibacillus | casei | |
| DMA 221 | SBI04006 | Bacillus | subtilis | | SBI04256 | Lacticaseibacillus | casei | |
| DMA 222 | SBI00461 | Bacillus | safensis | | SBI04256 | Lacticaseibacillus | casei | |
| DMA 223 | SBI04259 | Weisella | cibaria | | SBI00592 | Bacillus | mojavensis | |
| DMA 224 | SBI04259 | Weisella | cibaria | | SBI00714 | Bacillus | subtilis | |
| DMA 225 | SBI00714 | Bacillus | subtilis | | SBI04881 | Lentilactobacillus | buchneri | |
| DMA 226 | SBI04006 | Bacillus | subtilis | | SBI04881 | Lentilactobacillus | buchneri | |
| DMA 227 | SBI04881 | Lentilactobacillus | buchneri | | SBI00461 | Bacillus | safensis | |
| DMA 228 | SBI04006 | Bacillus | subtilis | | SBI04884 | Enterococcus | gilvus | |
| DMA 230 | SBI03599 | Bacillus | endophyticus | | SBI04887 | Lacticaseibacillus | casei | |
| DMA 231 | SBI00592 | Bacillus | mojavensis | | SBI04887 | Lacticaseibacillus | casei | |
| DMA 232 | SBI00693 | Bacillus | subtilis | | SBI04887 | Lacticaseibacillus | casei | |
| DMA 233 | SBI00714 | Bacillus | subtilis | | SBI04887 | Lacticaseibacillus | casei | |
| DMA 235 | SBI02181 | Bacillus | amyloliquefaciens | | SBI04887 | Lacticaseibacillus | casei | |
| DMA 236 | SBI04006 | Bacillus | subtilis | | SBI04887 | Lacticaseibacillus | casei | |
| DMA 237 | SBI00461 | Bacillus | safensis | | SBI04887 | Lacticaseibacillus | casei | |
| DMA 238 | SBI04874 | Lentilactobacillus | buchneri | | SBI00225 | Clavispora | sp. | |
| DMA 239 | CS00008 | Lactobacillus | acidophilus | La-14 | SBI01292 | Talaromyces | atroroseus | |
| DMA 240 | SBI04874 | Lentilactobacillus | buchneri | | SBI01292 | Talaromyces | atroroseus | |
| DMA 241 | CS00010 | Lacticaseibacillus | rhamnosus | Lr-32 | SBI01292 | Talaromyces | atroroseus | |
| DMA 242 | CS00008 | Lactobacillus | acidophilus | La-14 | CS00018 | Saccharomyces | boulardii | |
| DMA 243 | SBS04254 | Levilactobacillus | brevis | | CS00018 | Saccharomyces | boulardii | |
| DMA 244 | SBI05579 | Streptococcus | thermophilus | | SBI03599 | Bacillus | endophyticus | |
| DMA 245 | CS00001 | Bacillus | subtilis | DE111 | SBI04874 | Lentilactobacillus | buchneri | |
| DMA 246 | CS00008 | Lactobacillus | acidophilus | La-14 | SBI00589 | Bacillus | axarquiensis | |
| DMA 247 | SBS04254 | Levilactobacillus | brevis | | SBI00589 | Bacillus | axarquiensis | |
| DMA 248 | SBI04874 | Lentilactobacillus | buchneri | | SBI00589 | Bacillus | axarquiensis | |
| DMA 249 | SBI00598 | Bacillus | subtilis | | SBI04874 | Lentilactobacillus | buchneri | |
| DMA 250 | CS00008 | Lactobacillus | acidophilus | La-14 | SBI01595 | Bacillus | amyloliquefaciens | |
| DMA 251 | SBI01595 | Bacillus | amyloliquefaciens | | SBI04874 | Lentilactobacillus | buchneri | |
| DMA 252 | CS00008 | Lactobacillus | acidophilus | La-14 | SBI03602 | Bacillus | subtilis | |
| DMA 253 | CS00008 | Lactobacillus | acidophilus | La-14 | SBI04877 | Bacillus | amyloliquefaciens | |
| DMA 254 | SBI04877 | Bacillus | amyloliquefaciens | | SBI04874 | Lentilactobacillus | buchneri | |
| DMA 255 | CS00007 | Lactiplantibacillus | plantarum | 299V | SBI00461 | Bacillus | safensis | |
| DMA 256 | SBI01292 | Talaromyces | atroroseus | | CS00001 | Bacillus | subtilis | DE111 |
| DMA 258 | SBI04877 | Bacillus | amyloliquefaciens | | SBI01292 | Talaromyces | atroroseus | |
| DMA 260 | CS00001 | Bacillus | subtilis | DE111 | CS00018 | Saccharomyces | boulardii | |
| DMA 261 | SBI00589 | Bacillus | axarquiensis | | CS00018 | Saccharomyces | boulardii | |
| DMA 262 | SBI00598 | Bacillus | subtilis | | CS00018 | Saccharomyces | boulardii | |
| DMA 263 | SBI04877 | Bacillus | amyloliquefaciens | | CS00018 | Saccharomyces | boulardii | |
| DMA 264 | SBI00644 | Bacillus | aryabhattai | | CS00018 | Saccharomyces | boulardii | |
| DMA 265 | SBI05473 | Saccharomyces | cerevisiae | | SBI04916 | Lactococcus | lactis | |
| DMA 266 | SBS00319 | Debaryomyces | hansenii | | SBI00693 | Bacillus | subtilis | |
| DMA 267 | SBI03199 | Candida | dosseyi | | SBI00693 | Bacillus | subtilis | |
| DMA 268 | SBI05473 | Saccharomyces | cerevisiae | | SBI00693 | Bacillus | subtilis | |
| DMA 269 | SBI04932 | Galactomyces | geotrichum | | SBI00714 | Bacillus | subtilis | |
| DMA 270 | SBI05419 | Kazachstania | servazzii | | SBI00714 | Bacillus | subtilis | |
| DMA 271 | SBI05449 | Kluyveromyces | marxianus | | SBI00714 | Bacillus | subtilis | |
| DMA 272 | SBI00264 | Clavispora | sp. | | SBI00714 | Bacillus | subtilis | |
| DMA 273 | SBS00319 | Debaryomyces | hansenii | | SBI00714 | Bacillus | subtilis | |
| DMA 274 | SBI03199 | Candida | dosseyi | | SBI00714 | Bacillus | subtilis | |
| DMA 275 | CS00001 | Bacillus | subtilis | DE111 | SBI04873 | Pediococcus | ethanolidurans | |
| DMA 276 | SBI00589 | Bacillus | axarquiensis | | SBI04873 | Pediococcus | ethanolidurans | |
| DMA 277 | SBI00598 | Bacillus | subtilis | | SBI04873 | Pediococcus | ethanolidurans | |
| DMA 278 | SBI01595 | Bacillus | amyloliquefaciens | | SBI04873 | Pediococcus | ethanolidurans | |
| DMA 279 | SBI03602 | Bacillus | subtilis | | SBI04873 | Pediococcus | ethanolidurans | |
| DMA 280 | SBI04877 | Bacillus | amyloliquefaciens | | SBI04873 | Pediococcus | ethanolidurans | |
| DMA 282 | SBI05449 | Kluyveromyces | marxianus | | SBI02181 | Bacillus | amyloliquefaciens | |
| DMA 283 | SBI03199 | Candida | dosseyi | | SBI02181 | Bacillus | amyloliquefaciens | |
| DMA 284 | SBI05473 | Saccharomyces | cerevisiae | | SBI02181 | Bacillus | amyloliquefaciens | |
| DMA 285 | SBI00644 | Bacillus | aryabhattai | | SBI04874 | Lentilactobacillus | buchneri | |
| DMA 286 | SBI04932 | Galactomyces | geotrichum | | SBI02354 | Bacillus | pumilus | |
| DMA 287 | SBI05419 | Kazachstania | servazzii | | SBI02354 | Bacillus | pumilus | |
| DMA 288 | SBI05449 | Kluyveromyces | marxianus | | SBI02354 | Bacillus | pumilus | |
| DMA 289 | SBI00264 | Clavispora | sp. | | SBI02354 | Bacillus | pumilus | |
| DMA 290 | SBS00319 | Debaryomyces | hansenii | | SBI02354 | Bacillus | pumilus | |
| DMA 291 | SBI03199 | Candida | dosseyi | | SBI02354 | Bacillus | pumilus | |
| DMA 294 | SBI01681 | Candida | akabanensis | | SBI00592 | Bacillus | mojavensis | |
| DMA 295 | SBI05473 | Saccharomyces | cerevisiae | | SBI02354 | Bacillus | pumilus | |
| DMA 296 | SBI05469 | Nakazawaea | ishiwadae | | SBI00592 | Bacillus | mojavensis | |
| DMA 297 | SBI01681 | Candida | akabanensis | | SBI00693 | Bacillus | subtilis | |
| DMA 298 | SBI05469 | Nakazawaea | ishiwadae | | SBI00693 | Bacillus | subtilis | |
| DMA 299 | SBI01681 | Candida | akabanensis | | SBI00714 | Bacillus | subtilis | |
| DMA 300 | SBI05469 | Nakazawaea | ishiwadae | | SBI00714 | Bacillus | subtilis | |
| DMA 301 | SBI05469 | Nakazawaea | ishiwadae | | SBI02181 | Bacillus | amyloliquefaciens | |
| DMA 302 | SBI01681 | Candida | akabanensis | | SBI03602 | Bacillus | subtilis | |
| DMA 304 | SBI05469 | Nakazawaea | ishiwadae | | SBI03602 | Bacillus | subtilis | |
| DMA 305 | SBI00120 | Holtermanniella | takashimae | | SBI03602 | Bacillus | subtilis | |
| DMA 306 | SBI01681 | Candida | akabanensis | | SBI04006 | Bacillus | subtilis | |
| DMA 307 | SBI05469 | Nakazawaea | ishiwadae | | SBI04006 | Bacillus | subtilis | |
| DMA 308 | SBI01681 | Candida | akabanensis | | CS00001 | Bacillus | subtilis | DE111 |
| DMA 309 | SBI00264 | Clavispora | sp. | | SBI03599 | Bacillus | endophyticus | |
| DMA 310 | SBI05469 | Nakazawaea | ishiwadae | | CS00001 | Bacillus | subtilis | DE111 |
| DMA 311 | SBI00120 | Holtermanniella | takashimae | | CS00001 | Bacillus | subtilis | DE111 |
| DMA 313 | SBS00319 | Debaryomyces | hansenii | | SBI03599 | Bacillus | endophyticus | |
| DMA 314 | SBI05469 | Nakazawaea | ishiwadae | | SBI00589 | Bacillus | axarquiensis | |
| DMA 317 | SBI01681 | Candida | akabanensis | | SBI05579 | Streptococcus | thermophilus | |
| DMA 319 | SBS00319 | Debaryomyces | hansenii | | SBI05579 | Streptococcus | thermophilus | |
| DMA 320 | SBI03199 | Candida | dosseyi | | SBI05579 | Streptococcus | thermophilus | |
| DMA 321 | SBI05473 | Saccharomyces | cerevisiae | | CS00007 | Lactiplantibacillus | plantarum | 299V |
| DMA 322 | SBI01681 | Candida | akabanensis | | CS00007 | Lactiplantibacillus | plantarum | 299V |
| DMA 323 | SBS00208 | Pichia | terricola | | CS00007 | Lactiplantibacillus | plantarum | 299V |
| DMA 324 | SBS00319 | Debaryomyces | hansenii | | CS00007 | Lactiplantibacillus | plantarum | 299V |
| DMA 325 | SBI03199 | Candida | dosseyi | | CS00007 | Lactiplantibacillus | plantarum | 299V |
| DMA 326 | SBI05473 | Saccharomyces | cerevisiae | | SBI04918 | Lactococcus | lactis | |
| DMA 327 | SBI05473 | Saccharomyces | cerevisiae | | SBI04889 | Lactiplantibacillus | plantarum | |
| DMA 328 | SBI01681 | Candida | akabanensis | | SBI04889 | Lactiplantibacillus | plantarum | |
| DMA 329 | SBI03199 | Candida | dosseyi | | SBI04889 | Lactiplantibacillus | plantarum | |
| DMA 330 | SBI05473 | Saccharomyces | cerevisiae | | SBI04895 | Lacticaseibacillus | casei | |
| DMA 331 | SBI01681 | Candida | akabanensis | | SBI04895 | Lacticaseibacillus | casei | |
| DMA 332 | SBI03199 | Candida | dosseyi | | SBI04895 | Lacticaseibacillus | casei | |
| DMA 333 | SBI05473 | Saccharomyces | cerevisiae | | SBS04260 | Lactiplantibacillus | plantarum | |
| DMA 334 | SBI01681 | Candida | akabanensis | | SBS04260 | Lactiplantibacillus | plantarum | |
| DMA 335 | SBI01681 | Candida | akabanensis | | CS00008 | Lactobacillus | acidophilus | La-14 |
| DMA 336 | SBS00208 | Pichia | terricola | | CS00008 | Lactobacillus | acidophilus | La-14 |
| DMA 337 | SBS00319 | Debaryomyces | hansenii | | CS00008 | Lactobacillus | acidophilus | La-14 |
| DMA 338 | SBI03199 | Candida | dosseyi | | CS00008 | Lactobacillus | acidophilus | La-14 |
| DMA 339 | SBI01681 | Candida | akabanensis | | SBI04916 | Lactococcus | lactis | |
| DMA 340 | SBI03199 | Candida | dosseyi | | SBI04916 | Lactococcus | lactis | |
| DMA 341 | SBI05473 | Saccharomyces | cerevisiae | | SBI00598 | Bacillus | subtilis | |
| DMA 342 | SBI01681 | Candida | akabanensis | | SBI00598 | Bacillus | subtilis | |
| DMA 343 | SBI04932 | Galactomyces | geotrichum | | SBI03602 | Bacillus | subtilis | |
| DMA 344 | SBS00319 | Debaryomyces | hansenii | | SBI00598 | Bacillus | subtilis | |
| DMA 345 | SBI03199 | Candida | dosseyi | | SBI00598 | Bacillus | subtilis | |
| DMA 346 | SBI05473 | Saccharomyces | cerevisiae | | SBI00644 | Bacillus | aryabhattai | |
| DMA 348 | SBI05419 | Kazachstania | servazzii | | SBI03602 | Bacillus | subtilis | |
| DMA 349 | SBI05449 | Kluyveromyces | marxianus | | SBI03602 | Bacillus | subtilis | |
| DMA 350 | SBI00264 | Clavispora | sp. | | SBI03602 | Bacillus | subtilis | |
| DMA 351 | SBS00319 | Debaryomyces | hansenii | | SBI03602 | Bacillus | subtilis | |
| DMA 352 | SBI03199 | Candida | dosseyi | | SBI03602 | Bacillus | subtilis | |
| DMA 353 | SBI05449 | Kluyveromyces | marxianus | | SBI04006 | Bacillus | subtilis | |
| DMA 354 | SBI03199 | Candida | dosseyi | | SBI04006 | Bacillus | subtilis | |
| DMA 355 | SBI04932 | Galactomyces | geotrichum | | CS00001 | Bacillus | subtilis | DE111 |
| DMA 356 | SBI05449 | Kluyveromyces | marxianus | | CS00001 | Bacillus | subtilis | DE111 |
| DMA 357 | SBI03199 | Candida | dosseyi | | CS00001 | Bacillus | subtilis | DE111 |
| DMA 358 | SBI04932 | Galactomyces | geotrichum | | SBI04918 | Lactococcus | lactis | |
| DMA 359 | SBI05449 | Kluyveromyces | marxianus | | SBI04918 | Lactococcus | lactis | |
| DMA 360 | SBI00449 | Exiguobacterium | sp. | | SBI05473 | Saccharomyces | cerevisiae | |
| DMA 361 | CS00017 | Lacticaseibacillus | paracasei | Shirota | SBI00564 | Bacillus | subtilis | |
| DMA 362 | CS00017 | Lacticaseibacillus | paracasei | Shirota | SBI01595 | Bacillus | amyloliquefaciens | |
| DMA 363 | SBI04880 | Pediococcus | pentosaceus | | SBI05449 | Kluyveromyces | marxianus | |
| DMA 364 | CS00017 | Lacticaseibacillus | paracasei | Shirota | SBI05449 | Kluyveromyces | marxianus | |
| DMA 365 | SBI04880 | Pediococcus | pentosaceus | | CS00018 | Saccharomyces | boulardii | |
| DMA 366 | CS00017 | Lacticaseibacillus | paracasei | Shirota | CS00018 | Saccharomyces | boulardii | |
| DMA 369 | SBI00553 | Bacillus | subtilis | | SBI00817 | Pichia | fermentans | |
| DMA 370 | SBI00564 | Bacillus | subtilis | | SBI05473 | Saccharomyces | cerevisiae | |
| DMA 371 | SBI01595 | Bacillus | amyloliquefaciens | | SBI00817 | Pichia | fermentans | |
| DMA 372 | SBI01595 | Bacillus | amyloliquefaciens | | SBS04263 | Pichia | kudriavzevii | |
| DMA 373 | SBI01595 | Bacillus | amyloliquefaciens | | SBI05473 | Saccharomyces | cerevisiae | |
| DMA 375 | SBI04011 | Bacillus | megaterium | | SBI00817 | Pichia | fermentans | |
| DMA 376 | SBI04011 | Bacillus | megaterium | | SBI05473 | Saccharomyces | cerevisiae | |
| DMA 377 | CS00020 | Weizmannia | coagulans | BC30 | SBI00817 | Pichia | membranifaciens | |
| DMA 378 | CS00020 | Weizmannia | coagulans | BC30 | SBI05473 | Saccharomyces | cerevisiae | |
| DMA 380 | SBI05579 | Streptococcus | thermophilus | | SBI03602 | Bacillus | subtilis | |
| DMA 381 | SBI04259 | Weisella | cibaria | | SBI03602 | Bacillus | subtilis | |

TABLE 5
Growth Commensalism and Synergy in DMAs

| | | | | | | | | | Combination | Combination | Fold Change |
|---|---|---|---|---|---|---|---|---|---|---|---|
| | | | | | | | | | Growth Fold | Growth Fold | Over |
| DMA | Microbe | | | Strain | Microbe | | | Strain | Change | Change | Expected |
| # | #1 | Genus | Species | ID | #2 | Genus | Species | ID | Microbe #1 | Microbe #2 | Total Growth |
| DMA | SBS02335 | Pediococcus | pentosaceus | | SBI00461 | Bacillus | safensis | | 6153.85 | 0.92 | 2.15 |
| 23 | | | | | | | | | | | |
| DMA | SBS02335 | Pediococcus | pentosaceus | | SBI00589 | Bacillus | axarquiensis | | 2000 | 0.31 | 1.54 |
| 24 | | | | | | | | | | | |
| DMA | SBS04255 | Leuconostoc | mesenteroides | | SBI00461 | Bacillus | safensis | | 3846.15 | 0.37 | 3 |
| 26 | | | | | | | | | | | |
| DMA | SBS04255 | Leuconostoc | mesenteroides | | SBI00589 | Bacillus | axarquiensis | | 1500 | 1.95 | 5.09 |
| 27 | | | | | | | | | | | |
| DMA | SBS04255 | Leuconostoc | mesenteroides | | SBI00598 | Bacillus | subtilis | | 5384.62 | 3.68 | 7.36 |
| 28 | | | | | | | | | | | |
| DMA | SBS04255 | Leuconostoc | mesenteroides | | SBI05473 | Saccharomyces | cerevisiae | | 0.69 | 2.63 | 2.62 |
| 29 | | | | | | | | | | | |
| DMA | SBI04919 | Pediococcus | parvulus | | SBI00461 | Bacillus | safensis | | 53.85 | 7.78 | 35 |
| 30 | | | | | | | | | | | |
| DMA | SBI05399 | Lactiplantibacillus | plantarum | | SBI00461 | Bacillus | safensis | | 692.31 | 1.89 | 2.88 |
| 31 | | | | | | | | | | | |
| DMA | SBI05399 | Lactiplantibacillus | plantarum | | SBI00598 | Bacillus | subtilis | | 923.08 | 6.67 | 7.99 |
| 32 | | | | | | | | | | | |
| DMA | SBI05399 | Lactiplantibacillus | plantarum | | SBI05473 | Saccharomyces | cerevisiae | | 0.44 | 7.78 | 7.65 |
| 33 | | | | | | | | | | | |
| DMA | SBI04256 | Lacticaseibacillus | casei | | SBI00817 | Pichia | fermentans | | 0.44 | 25.71 | 25.71 |
| 34 | | | | | | | | | | | |
| DMA | SBI04895 | Lacticaseibacillus | casei | | SBI00817 | Pichia | fermentans | | 0.31 | 23.81 | 23.81 |
| 38 | | | | | | | | | | | |
| DMA | SBI04887 | Lacticaseibacillus | casei | | SBS04263 | Pichia | kudriavzevii | | 0.3 | 56521.74 | 48148.19 |
| 43 | | | | | | | | | | | |
| DMA | SBI04889 | Lactiplantibacillus | plantarum | | SBS04263 | Pichia | kudriavzevii | | 2.25 | 100 | 99.98 |
| 44 | | | | | | | | | | | |
| DMA | SBI04895 | Lacticaseibacillus | casei | | SBS04263 | Pichia | kudriavzevii | | 0.33 | 2.86 | 2.86 |
| 45 | | | | | | | | | | | |
| DMA | SBI04887 | Lacticaseibacillus | casei | | SBI00997 | Bacillus | safensis | | 1.09 | 652.17 | 326.63 |
| 48 | | | | | | | | | | | |
| DMA | SBI04887 | Lacticaseibacillus | casei | | SBI01819 | Bacillus | pumilus | | 62.5 | 0.3 | 16.35 |
| 49 | | | | | | | | | | | |
| DMA | SBI04887 | Lacticaseibacillus | casei | | SBI04173 | Bacillus | paralicheniformis | | 2 | 39.13 | 21.86 |
| 53 | | | | | | | | | | | |
| DMA | SBI04895 | Lacticaseibacillus | casei | | SBI04173 | Bacillus | paralicheniformis | | 0.35 | 4.29 | 4.28 |
| 55 | | | | | | | | | | | |
| DMA | SBI01819 | Bacillus | pumilus | | SBI04910 | Pediococcus | ethanolidurans | | 0.53 | 25 | 8.36 |
| 64 | | | | | | | | | | | |
| DMA | SBI02185 | Bacillus | licheniformis | | SBI04910 | Pediococcus | ethanolidurans | | 88.24 | 0.57 | 17.7 |
| 65 | | | | | | | | | | | |
| DMA | SBI04173 | Bacillus | paralicheniformis | | SBI04910 | Pediococcus | ethanolidurans | | 29.41 | 0.5 | 13.78 |
| 66 | | | | | | | | | | | |
| DMA | SBI04877 | Bacillus | amyloliquefaciens | | SBI04910 | Pediococcus | ethanolidurans | | 1 | 571.43 | 167.38 |
| 68 | | | | | | | | | | | |
| DMA | SBI01819 | Bacillus | pumilus | | SBI04913 | Schleiferilactobacillus | harbinensis | | 0.79 | 112.5 | 33.89 |
| 71 | | | | | | | | | | | |
| DMA | SBI02185 | Bacillus | licheniformis | | SBI04913 | Schleiferilactobacillus | harbinensis | | 42.11 | 0.56 | 9.43 |
| 72 | | | | | | | | | | | |
| DMA | SBI04173 | Bacillus | paralicheniformis | | SBI04913 | Schleiferilactobacillus | harbinensis | | 5.26 | 1.5 | 3.33 |
| 73 | | | | | | | | | | | |
| DMA | SBI04877 | Bacillus | amyloliquefaciens | | SBI04913 | Schleiferilactobacillus | harbinensis | | 4.74 | 5714.29 | 1541.92 |
| 75 | | | | | | | | | | | |
| DMA | SBI00997 | Bacillus | safensis | | SBI04916 | Lactococcus | lactis | | 11.36 | 0.39 | 11.36 |
| 76 | | | | | | | | | | | |
| DMA | SBI04874 | Lentilactobacillus | buchneri | | CS00018 | Saccharomyces | boulardii | | 25 | 4 | 4.81 |
| 85 | | | | | | | | | | | |
| DMA | SBI04874 | Lentilactobacillus | buchneri | | SBI05449 | Kluyveromyces | marxianus | | 300 | 1.4 | 1.7 |
| 87 | | | | | | | | | | | |
| DMA | SBI04874 | Lentilactobacillus | buchneri | | SBI00553 | Bacillus | subtilis | | 50 | 1.4 | 7.37 |
| 88 | | | | | | | | | | | |
| DMA | SBI04873 | Pediococcus | ethanolidurans | | CS00018 | Saccharomyces | boulardii | | 5 | 666.67 | 19.56 |
| 92 | | | | | | | | | | | |
| DMA | SBI04873 | Pediococcus | ethanolidurans | | SBI05449 | Kluyveromyces | marxianus | | 800 | 1111.11 | 947.37 |
| 93 | | | | | | | | | | | |
| DMA | SBI04873 | Pediococcus | ethanolidurans | | SBI00553 | Bacillus | subtilis | | 35.71 | 122.22 | 36.27 |
| 94 | | | | | | | | | | | |
| DMA | SBI04873 | Pediococcus | ethanolidurans | | SBI00564 | Bacillus | subtilis | | 10.67 | 66.67 | 11 |
| 97 | | | | | | | | | | | |
| DMA | CS00010 | Lacticaseibacillus | rhamnosus | Lr-32 | SBI05449 | Kluyveromyces | marxianus | | 300 | 1.03 | 1.03 |
| 99 | | | | | | | | | | | |
| DMA | SBI04910 | Pediococcus | ethanolidurans | | CS00018 | Saccharomyces | boulardii | | 1.5 | 11.25 | 3.13 |
| 101 | | | | | | | | | | | |
| DMA | SBI04910 | Pediococcus | ethanolidurans | | SBI05449 | Kluyveromyces | marxianus | | 30 | 3.75 | 6.67 |
| 103 | | | | | | | | | | | |
| DMA | SBI04910 | Pediococcus | ethanolidurans | | SBI00553 | Bacillus | subtilis | | 5 | 12.5 | 5.41 |
| 104 | | | | | | | | | | | |
| DMA | SBI04910 | Pediococcus | ethanolidurans | | SBI00564 | Bacillus | subtilis | | 1.93 | 10 | 2.34 |
| 107 | | | | | | | | | | | |
| DMA | SBI04913 | Lactobacillus | harbinensis | | CS00018 | Saccharomyces | boulardii | | 0.5 | 7.69 | 2.26 |
| 108 | | | | | | | | | | | |
| DMA | SBI04913 | Schleiferilactobacillus | harbinensis | | SBI05449 | Kluyveromyces | marxianus | | 50 | 5.38 | 8.57 |
| 109 | | | | | | | | | | | |
| DMA | SBI04913 | Schleiferilactobacillus | harbinensis | | SBI00553 | Bacillus | subtilis | | 8.57 | 3.08 | 8.1 |
| 110 | | | | | | | | | | | |
| DMA | SBI05697 | Lactiplantibacillus | plantarum | | CS00018 | Saccharomyces | boulardii | | 3 | 18.46 | 18.46 |
| 114 | | | | | | | | | | | |
| DMA | SBI05697 | Lactiplantibacillus | plantarum | | SBI05449 | Kluyveromyces | marxianus | | 80 | 17.69 | 17.69 |
| 117 | | | | | | | | | | | |
| DMA | SBI00553 | Bacillus | subtilis | | SBI04881 | Lentilactobacillus | buchneri | | 183.33 | 78.57 | 110 |
| 124 | | | | | | | | | | | |
| DMA | SBI00449 | Exiguobacterium | sp. | | CS00018 | Saccharomyces | boulardii | | 2.75 | 666.67 | 208.79 |
| 129 | | | | | | | | | | | |
| DMA | SBI00449 | Exiguobacterium | sp. | | SBI05449 | Kluyveromyces | marxianus | | 300 | 500 | 489.47 |
| 130 | | | | | | | | | | | |
| DMA | SBI00449 | Exiguobacterium | sp. | | SBI04881 | Lentilactobacillus | buchneri | | 1666.67 | 55.56 | 1294.87 |
| 132 | | | | | | | | | | | |
| DMA | SBI00449 | Exiguobacterium | sp. | | SBI04919 | Pediococcus | parvulus | | 0.33 | 50 | 18.96 |
| 133 | | | | | | | | | | | |
| DMA | SBI00564 | Bacillus | subtilis | | SBI04881 | Lentilactobacillus | buchneri | | 2166.67 | 106.67 | 695.24 |
| 141 | | | | | | | | | | | |
| DMA | SBI00564 | Bacillus | subtilis | | SBI04919 | Pediococcus | parvulus | | 266.67 | 12 | 54.44 |
| 142 | | | | | | | | | | | |
| DMA | SBI01595 | Bacillus | amyloliquefaciens | | SBI05449 | Kluyveromyces | marxianus | | 13 | 10 | 10.3 |
| 148 | | | | | | | | | | | |
| DMA | SBI01595 | Bacillus | amyloliquefaciens | | SBI04919 | Pediococcus | parvulus | | 43.33 | 1.67 | 33.72 |
| 152 | | | | | | | | | | | |
| DMA | SBI01187 | Pichia | membranifaciens | | SBI00714 | Bacillus | subtilis | | 10 | 0.6 | 5.3 |
| 161 | | | | | | | | | | | |
| DMA | SBI02945 | Meyerozyma | guillermondii | | SBI00714 | Bacillus | subtilis | | 10 | 0.67 | 4.91 |
| 164 | | | | | | | | | | | |
| DMA | SBS04263 | Pichia | kudriavzevii | | SBI00714 | Bacillus | subtilis | | 8 | 0.38 | 3.31 |
| 165 | | | | | | | | | | | |
| DMA | SBS04263 | Pichia | kudriavzevii | | SBI02181 | Bacillus | amyloliquefaciens | | 10 | 0.75 | 1.19 |
| 167 | | | | | | | | | | | |
| DMA | SBI01679 | Candida | sp. | | SBI02354 | Bacillus | pumilus | | 18571.43 | 1.25 | 18570.37 |
| 170 | | | | | | | | | | | |
| DMA | SBS04263 | Pichia | kudriavzevii | | SBI02354 | Bacillus | pumilus | | 7.14 | 0.38 | 3.53 |
| 172 | | | | | | | | | | | |
| DMA | SBS04263 | Pichia | kudriavzevii | | CS00001 | Bacillus | subtilis | DE111 | 3000 | 0.63 | 4.37 |
| 182 | | | | | | | | | | | |
| DMA | SBI05449 | Kluyveromyces | marxianus | | SBI00592 | Bacillus | mojavensis | | 10 | 0.5 | 0.95 |
| 186 | | | | | | | | | | | |
| DMA | SBI05449 | Kluyveromyces | marxianus | | SBI00693 | Bacillus | subtilis | | 3.67 | 0.9 | 1.54 |
| 193 | | | | | | | | | | | |
| DMA | SBS04254 | Levilactobacillus | brevis | | SBI00120 | Holtermanniella | takashimae | | 0.75 | 200 | 196.64 |
| 195 | | | | | | | | | | | |
| DMA | SBI00120 | Kwoniella | shandongens | | SBI00693 | Bacillus | subtilis | | 6.67 | 0.56 | 6.49 |
| 208 | | | | | | | | | | | |
| DMA | SBI00120 | Kwoniella | shandongens | | SBI04006 | Bacillus | subtilis | | 60 | 0.44 | 21.71 |
| 211 | | | | | | | | | | | |
| DMA | SBI04881 | Lentilactobacillus | buchneri | | SBI00461 | Bacillus | safensis | | 16153.85 | 1.38 | 3 |
| 227 | | | | | | | | | | | |
| DMA | SBI03599 | Bacillus | endophyticus | | SBI04887 | Lacticaseibacillus | casei | | 6.11 | 300 | 48.1 |
| 230 | | | | | | | | | | | |
| DMA | SBI02181 | Bacillus | amyloliquefaciens | | SBI04887 | Lacticaseibacillus | casei | | 3.89 | 175 | 4.65 |
| 235 | | | | | | | | | | | |
| DMA | SBI04006 | Bacillus | subtilis | | SBI04887 | Lacticaseibacillus | casei | | 2.78 | 3000 | 4.44 |
| 236 | | | | | | | | | | | |
| DMA | SBI04874 | Lentilactobacillus | buchneri | | SBI01292 | Talaromyces | atroroseus | | 0.55 | 750 | 749.81 |
| 240 | | | | | | | | | | | |
| DMA | CS00008 | Lactobacillus | acidophilus | La-14 | CS00018 | Saccharomyces | boulardii | | 4.29 | 1125 | 5.57 |
| 242 | | | | | | | | | | | |
| DMA | SBI04874 | Lentilactobacillus | buchneri | | CS00001 | Bacillus | subtilis | DE111 | 1.29 | 25 | 13.93 |
| 245 | | | | | | | | | | | |
| DMA | CS00008 | Lactobacillus | acidophilus | La-14 | SBI00589 | Bacillus | axarquiensis | | 2.86 | 1.63 | 2.84 |
| 246 | | | | | | | | | | | |
| DMA | SBI04874 | Lentilactobacillus | buchneri | | SBI00589 | Bacillus | axarquiensis | | 0.57 | 12.5 | 11.54 |
| 248 | | | | | | | | | | | |
| DMA | CS00008 | Lactobacillus | acidophilus | La-14 | SBI01595 | Bacillus | amyloliquefaciens | | 6.67 | 1125 | 138.24 |
| 250 | | | | | | | | | | | |
| DMA | SBI01595 | Bacillus | amyloliquefaciens | | SBI04874 | Lentilactobacillus | buchneri | | 3625 | 0.5 | 3598.02 |
| 251 | | | | | | | | | | | |
| DMA | CS00008 | Lactobacillus | acidophilus | La-14 | SBI03602 | Bacillus | subtilis | | 5 | 10000 | 5716.43 |
| 252 | | | | | | | | | | | |
| DMA | CS00008 | Lactobacillus | acidophilus | La-14 | SBI04877 | Bacillus | amyloliquefaciens | | 5 | 26250 | 3092.65 |
| 253 | | | | | | | | | | | |
| DMA | SBI04877 | Bacillus | amyloliquefaciens | | SBI04874 | Lentilactobacillus | buchneri | | 1875 | 6.67 | 1861.09 |
| 254 | | | | | | | | | | | |
| DMA | SBI04932 | Galactomyces | geotrichum | | SBI00714 | Bacillus | subtilis | | 6 | 2 | 5.98 |
| 269 | | | | | | | | | | | |
| DMA | SBI05449 | Kluyveromyces | marxianus | | SBI00714 | Bacillus | subtilis | | 6 | 0.6 | 0.86 |
| 271 | | | | | | | | | | | |
| DMA | CS00001 | Bacillus | subtilis | DE111 | SBI04873 | Pediococcus | ethanolidurans | | 66666.67 | 1.71 | 30.27 |
| 275 | | | | | | | | | | | |
| DMA | SBI03602 | Bacillus | subtilis | | SBI04873 | Pediococcus | ethanolidurans | | 333.33 | 833.33 | 666.67 |
| 279 | | | | | | | | | | | |
| DMA | SBI04877 | Bacillus | amyloliquefaciens | | SBI04873 | Pediococcus | ethanolidurans | | 1333333.33 | 10 | 63501.59 |
| 280 | | | | | | | | | | | |
| DMA | SBI05449 | Kluyveromyces | marxianus | | SBI02181 | Bacillus | amyloliquefaciens | | 1750 | 0.9 | 7.87 |
| 282 | | | | | | | | | | | |
| DMA | SBI04932 | Galactomyces | geotrichum | | SBI02354 | Bacillus | pumilus | | 10000 | 1.5 | 9971.51 |
| 286 | | | | | | | | | | | |
| DMA | SBI05419 | Kazachstania | servazzii | | SBI02354 | Bacillus | pumilus | | 42.86 | 0.37 | 30.11 |
| 287 | | | | | | | | | | | |
| DMA | SBI05473 | Saccharomyces | cerevisiae | | SBI02354 | Bacillus | pumilus | | 42.86 | 1.67 | 9.46 |
| 295 | | | | | | | | | | | |
| DMA | SBI05469 | Nakazawaea | ishiwadae | | SBI00592 | Bacillus | mojavensis | | 8.33 | 3.33 | 7.68 |
| 296 | | | | | | | | | | | |
| DMA | SBI01681 | Candida | akabanensis | | SBI00693 | Bacillus | subtilis | | 6.67 | 0.45 | 6.45 |
| 297 | | | | | | | | | | | |
| DMA | SBI05469 | Nakazawaea | ishiwadae | | SBI02181 | Bacillus | amyloliquefaciens | | 15 | 0.89 | 5.23 |
| 301 | | | | | | | | | | | |
| DMA | SBI01681 | Candida | akabanensis | | SBI04006 | Bacillus | subtilis | | 46 | 0.36 | 14.63 |
| 306 | | | | | | | | | | | |
| DMA | SBI05469 | Nakazawaea | ishiwadae | | SBI04006 | Bacillus | subtilis | | 14 | 1.11 | 5.71 |
| 307 | | | | | | | | | | | |
| DMA | SBI05469 | Nakazawaea | ishiwadae | | CS00001 | Bacillus | subtilis | DE111 | 6.67 | 1 | 1.18 |
| 310 | | | | | | | | | | | |
| DMA | SBI00120 | Holtermanniella | takashimae | | CS00001 | Bacillus | subtilis | DE111 | 13.33 | 0.89 | 1.29 |
| 311 | | | | | | | | | | | |
| DMA | SBI05473 | Saccharomyces | cerevisiae | | CS00007 | Lactiplantibacillus | plantarum | 299V | 6.57 | 0.5 | 6.57 |
| 321 | | | | | | | | | | | |
| DMA | SBI05473 | Saccharomyces | cerevisiae | | SBI04889 | Lactiplantibacillus | plantarum | | 29 | 0.33 | 28.89 |
| 327 | | | | | | | | | | | |
| DMA | SBI05473 | Saccharomyces | cerevisiae | | SBS04260 | Lactiplantibacillus | plantarum | | 82 | 1 | 81.36 |
| 333 | | | | | | | | | | | |
| DMA | SBI05473 | Saccharomyces | cerevisiae | | SBI00598 | Bacillus | subtilis | | 71.43 | 0.5 | 11.06 |
| 341 | | | | | | | | | | | |
| DMA | SBI05449 | Kluyveromyces | marxianus | | SBI03602 | Bacillus | subtilis | | 166.67 | 0.8 | 1.3 |
| 349 | | | | | | | | | | | |
| DMA | SBI05449 | Kluyveromyces | marxianus | | SBI04006 | Bacillus | subtilis | | 26.67 | 0.8 | 1.55 |
| 353 | | | | | | | | | | | |
| DMA | SBI04932 | Galactomyces | geotrichum | | CS00001 | Bacillus | subtilis | DE111 | 300 | 1.5 | 101 |
| 355 | | | | | | | | | | | |
| DMA | SBI05449 | Kluyveromyces | marxianus | | CS00001 | Bacillus | subtilis | DE111 | 2000 | 0.5 | 0.7 |
| 356 | | | | | | | | | | | |
| DMA | SBI04932 | Galactomyces | geotrichum | | SBI04918 | Lactococcus | lactis | | 4.77 | 10 | 4.77 |
| 358 | | | | | | | | | | | |
| DMA | SBI05449 | Kluyveromyces | marxianus | | SBI04918 | Lactococcus | lactis | | 4.92 | 0.4 | 4.92 |
| 359 | | | | | | | | | | | |
| DMA | SBI04880 | Pediococcus | pentosaceus | | SBI05449 | Kluyveromyces | marxianus | | 0.5 | 27894.74 | 18276.03 |
| 363 | | | | | | | | | | | |
| DMA | CS00017 | Lacticaseibacillus | paracasei | Shirota | SBI05449 | Kluyveromyces | marxianus | | 0.6 | 266.67 | 258.08 |
| 364 | | | | | | | | | | | |
| DMA | SBI04880 | Pediococcus | pentosaceus | | CS00018 | Saccharomyces | boulardii | | 0.6 | 15263.16 | 10000.21 |
| 365 | | | | | | | | | | | |
| DMA | CS00017 | Lacticaseibacillus | paracasei | Shirota | CS00018 | Saccharomyces | boulardii | | 0.9 | 166.67 | 161.32 |
| 366 | | | | | | | | | | | |
| DMA | SBI00564 | Bacillus | subtilis | | SBI05473 | Saccharomyces | cerevisiae | | 3.33 | 0.27 | 1.29 |
| 370 | | | | | | | | | | | |
| DMA | SBI00120 | Kwoniella | shandongens | | SBI02181 | Bacillus | amyloliquefaciens | | 27.5 | 0.78 | 9 |
| 210 | | | | | | | | | | | |
| DMA | SBI01187 | Pichia | membranifaciens | | SBI02181 | Bacillus | amyloliquefaciens | | 75 | 0.6 | 6.11 |
| 166 | | | | | | | | | | | |
| DMA | SBI01595 | Bacillus | amyloliquefaciens | | SBI05473 | Saccharomyces | cerevisiae | | 4.67 | 0.6 | 1.54 |
| 373 | | | | | | | | | | | |

TABLE 6
Indole Production Additivity and Synergy
(0 = No indole production, 1 = Potential Indole production, 2 = Indole production,
3 = Robust indole production, 4 = Very Robust indole production)

| | | | | | | | | | | | Predic- | |
|---|---|---|---|---|---|---|---|---|---|---|---|---|
| | | | | | Strain | | | | | Strain | ted | Combin- |
| | | | | | #1 | | | | | #2 | Combin- | ation |
| DMA | Strain | | | Strain | Indole | Strain | | | Strain | Indole | ation | Final |
| # | #1 | Genus | Species | ID | Score | #2 | Genus | Species | ID | Score | Score | Score |
| DMA | SBI04887 | Lacticasei- | casei | | 0 | SBI02185 | Bacillus | licheniformis | | 1 | 1 | 3 |
| 51 | | bacillus | | | | | | | | | | |
| DMA | SBI04889 | Lactiplanti- | plantarum | | 0 | SBI02185 | Bacillus | licheniformis | | 1 | 1 | 3 |
| 52 | | bacillus | | | | | | | | | | |
| DMA | SBI04887 | Lacticasei- | casei | | 0 | SBI04173 | Bacillus | paralicheni- | | 1 | 1 | 3 |
| 53 | | bacillus | | | | | | formis | | | | |
| DMA | SBI04889 | Lactiplanti- | plantarum | | 0 | SBI04173 | Bacillus | paralicheni- | | 1 | 1 | 3 |
| 54 | | bacillus | | | | | | formis | | | | |
| DMA | SBI04902 | Pediococcus | pentosaceus | | 0 | SBI00553 | Bacillus | subtilis | | 1 | 1 | 3 |
| 100 | | | | | | | | | | | | |
| DMA | SBI05449 | Kluyveromyces | marxianus | | 0 | SBI03599 | Bacillus | endophyticus | | 2 | 2 | 3 |
| 128 | | | | | | | | | | | | |
| DMA | SBI00564 | Bacillus | subtilis | | 1 | SBI05419 | Kazachstania | servazzii | | 0 | 1 | 2 |
| 138 | | | | | | | | | | | | |
| DMA | SBI00564 | Bacillus | subtilis | | 1 | SBI05449 | Kluyvero- | marxianus | | 0 | 1 | 2 |
| 139 | | | | | | | myces | | | | | |
| DMA | SBI00564 | Bacillus | subtilis | | 1 | SBS04255 | Leuconostoc | mesenteroides | | 0 | 1 | 2 |
| 140 | | | | | | | | | | | | |
| DMA | SBI00564 | Bacillus | subtilis | | 1 | SBI04919 | Pediococcus | parvulus | | 0 | 1 | 2 |
| 142 | | | | | | | | | | | | |
| DMA | SBI00564 | Bacillus | subtilis | | 1 | SBI04900 | Leuconostoc | pseudo- | | 0 | 1 | 2 |
| 143 | | | | | | | | mesenteroides | | | | |
| DMA | SBI00564 | Bacillus | subtilis | | 1 | SBI05399 | Lentilacto- | plantarum | | 0 | 1 | 3 |
| 379 | | | | | | | bacillus | | | | | |
| DMA | SBI01595 | Bacillus | vallismortis | | 1 | SBI05419 | Kazachstania | servazzii | | 0 | 1 | 3 |
| 147 | | | | | | | | | | | | |
| DMA | SBI01595 | Bacillus | vallismortis | | 1 | SBI05469 | Nakazawaea | ishiwadae | | 0 | 1 | 2 |
| 149 | | | | | | | | | | | | |
| DMA | SBI01595 | Bacillus | vallismortis | | 1 | SBS02335 | Pediococcus | pentosaceus | | 0 | 1 | 2 |
| 150 | | | | | | | | | | | | |
| DMA | SBI01595 | Bacillus | vallismortis | | 1 | SBI04881 | Lentilacto- | buchneri | | 0 | 1 | 2 |
| 151 | | | | | | | bacillus | | | | | |
| DMA | SBI01595 | Bacillus | vallismortis | | 1 | SBI04919 | Pediococcus | parvulus | | 0 | 1 | 2 |
| 152 | | | | | | | | | | | | |
| DMA | SBI01595 | Bacillus | vallismortis | | 1 | SBI04900 | Leuconostoc | pseudomese | | 0 | 1 | 2 |
| 153 | | | | | | | | nteroides | | | | |
| DMA | SBI01679 | Candida | sp. | | 0 | SBI00592 | Bacillus | mojavensis | | 2 | 2 | 3 |
| 155 | | | | | | | | | | | | |
| DMA | SBI01683 | Pichia | fermentans | | 0 | SBI00592 | Bacillus | mojavensis | | 2 | 2 | 3 |
| 156 | | | | | | | | | | | | |
| DMA | SBS04263 | Pichia | kudriavzevii | | 0 | SBI00592 | Bacillus | mojavensis | | 2 | 2 | 3 |
| 157 | | | | | | | | | | | | |
| DMA | SBI01679 | Candida | sp. | | 0 | SBI00693 | Bacillus | subtilis | | 2 | 2 | 3 |
| 158 | | | | | | | | | | | | |
| DMA | SBI01683 | Pichia | fermentans | | 0 | SBI00693 | Bacillus | subtilis | | 2 | 2 | 3 |
| 159 | | | | | | | | | | | | |
| DMA | SBS04263 | Pichia | kudriavzevii | | 0 | SBI00693 | Bacillus | subtilis | | 2 | 2 | 3 |
| 160 | | | | | | | | | | | | |
| DMA | SBI01679 | Candida | sp. | | 0 | SBI00714 | Bacillus | subtilis | | 2 | 2 | 3 |
| 162 | | | | | | | | | | | | |
| DMA | SBI01683 | Pichia | fermentans | | 0 | SBI00714 | Bacillus | subtilis | | 2 | 2 | 3 |
| 163 | | | | | | | | | | | | |
| DMA | SBS04263 | Pichia | kudriavzevii | | 0 | SBI00714 | Bacillus | subtilis | | 2 | 2 | 3 |
| 165 | | | | | | | | | | | | |
| DMA | SBI01187 | Pichia | membranifaciens | | 2 | SBI02354 | Bacillus | pumilus | | 1 | 3 | 3 |
| 168 | | | | | | | | | | | | |
| DMA | SBI01292 | Talaromyces | atroroseus | | 0 | SBI02354 | Bacillus | pumilus | | 1 | 1 | 3 |
| 169 | | | | | | | | | | | | |
| DMA | SBI01679 | Candida | sp. | | 0 | SBI02354 | Bacillus | pumilus | | 1 | 1 | 2 |
| 170 | | | | | | | | | | | | |
| DMA | SBI01683 | Pichia | fermentans | | 0 | SBI02354 | Bacillus | pumilus | | 1 | 1 | 2 |
| 171 | | | | | | | | | | | | |
| DMA | SBI01187 | Pichia | membranifaciens | | 2 | SBI04006 | Bacillus | subtilis | | 2 | 4 | 3 |
| 179 | | | | | | | | | | | | |
| DMA | SBI04932 | Galactomyces | geotrichum | | 0 | SBI00592 | Bacillus | mojavensis | | 2 | 2 | 3 |
| 184 | | | | | | | | | | | | |
| DMA | SBI05419 | Kazachstania | servazzii | | 0 | SBI00592 | Bacillus | mojavensis | | 2 | 2 | 3 |
| 185 | | | | | | | | | | | | |
| DMA | SBI05449 | Kluyveromyces | marxianus | | 0 | SBI00592 | Bacillus | mojavensis | | 2 | 2 | 2 |
| 186 | | | | | | | | | | | | |
| DMA | SBI00264 | Clavispora | sp. | | 0 | SBI00592 | Bacillus | mojavensis | | 2 | 2 | 3 |
| 187 | | | | | | | | | | | | |
| DMA | SBS00319 | Debaryomyces | hansenii | | 0 | SBI00592 | Bacillus | mojavensis | | 2 | 2 | 3 |
| 188 | | | | | | | | | | | | |
| DMA | SBI03199 | Candida | dosseyi | | 0 | SBI00592 | Bacillus | mojavensis | | 2 | 2 | 3 |
| 189 | | | | | | | | | | | | |
| DMA | SBI05473 | Saccharomyces | cerevisiae | | 0 | SBI00592 | Bacillus | mojavensis | | 2 | 2 | 3 |
| 190 | | | | | | | | | | | | |
| DMA | SBI04932 | Galactomyces | geotrichum | | 0 | SBI00693 | Bacillus | subtilis | | 2 | 2 | 3 |
| 191 | | | | | | | | | | | | |
| DMA | SBI05419 | Kazachstania | servazzii | | 0 | SBI00693 | Bacillus | subtilis | | 2 | 2 | 3 |
| 192 | | | | | | | | | | | | |
| DMA | SBI05449 | Kluyveromyces | marxianus | | 0 | SBI00693 | Bacillus | subtilis | | 2 | 2 | 3 |
| 193 | | | | | | | | | | | | |
| DMA | SBI00264 | Clavispora | sp. | | 0 | SBI00693 | Bacillus | subtilis | | 2 | 2 | 3 |
| 194 | | | | | | | | | | | | |
| DMA | SBI03599 | Bacillus | endophyticus | | 2 | SBI04916 | Lactococcus | lactis | | 0 | 2 | 3 |
| 200 | | | | | | | | | | | | |
| DMA | SBS04260 | Lactiplanti- | plantarum | | 0 | SBI00592 | Bacillus | mojavensis | | 2 | 2 | 3 |
| 202 | | bacillus | | | | | | | | | | |
| DMA | SBS04260 | Lactiplanti- | plantarum | | 0 | SBI00693 | Bacillus | subtilis | | 2 | 2 | 3 |
| 203 | | bacillus | | | | | | | | | | |
| DMA | SBS04260 | Lactiplanti- | plantarum | | 0 | SBI04006 | Bacillus | subtilis | | 2 | 2 | 3 |
| 206 | | bacillus | | | | | | | | | | |
| DMA | SBI00120 | Holtermanniella | takashimae | | 0 | SBI00592 | Bacillus | mojavensis | | 2 | 2 | 3 |
| 207 | | | | | | | | | | | | |
| DMA | SBI00120 | Holtermanniella | takashimae | | 0 | SBI00693 | Bacillus | subtilis | | 2 | 2 | 3 |
| 208 | | | | | | | | | | | | |
| DMA | SBI00120 | Holtermanniella | takashimae | | 0 | SBI00714 | Bacillus | subtilis | | 2 | 2 | 3 |
| 209 | | | | | | | | | | | | |
| DMA | SBI01292 | Talaromyces | atroroseus | | 0 | SBI00592 | Bacillus | mojavensis | | 2 | 2 | 3 |
| 214 | | | | | | | | | | | | |
| DMA | SBI01292 | Talaromyces | atroroseus | | 0 | SBI00693 | Bacillus | subtilis | | 2 | 2 | 3 |
| 215 | | | | | | | | | | | | |
| DMA | SBI01292 | Talaromyces | atroroseus | | 0 | SBI00714 | Bacillus | subtilis | | 2 | 2 | 3 |
| 216 | | | | | | | | | | | | |
| DMA | SBI01292 | Talaromyces | atroroseus | | 0 | SBI04006 | Bacillus | subtilis | | 2 | 2 | 3 |
| 217 | | | | | | | | | | | | |
| DMA | SBI04259 | Weisella | cibaria | | 0 | SBI00592 | Bacillus | mojavensis | | 2 | 2 | 3 |
| 223 | | | | | | | | | | | | |
| DMA | SBI04259 | Weisella | cibaria | | 0 | SBI00714 | Bacillus | subtilis | | 2 | 2 | 3 |
| 224 | | | | | | | | | | | | |
| DMA | SBI03599 | Bacillus | endophyticus | | 2 | SBI04887 | Lacticasei- | casei | | 0 | 2 | 3 |
| 230 | | | | | | | bacillus | | | | | |
| DMA | SBI04006 | Bacillus | subtilis | | 2 | SBI04887 | Lacticasei- | casei | | 0 | 2 | 3 |
| 236 | | | | | | | bacillus | | | | | |
| DMA | CS00001 | Bacillus | subtilis | DE111 | 1 | SBI04874 | Lacticasei- | buchneri | | 0 | 1 | 2 |
| 245 | | | | | | | bacillus | | | | | |
| DMA | CS00008 | Lactobacillus | acidophilus | La-14 | 1 | SBI00589 | Bacillus | axarquiensis | | 1 | 2 | 2 |
| 246 | | | | | | | | | | | | |
| DMA | SBS04254 | Levilacto- | brevis | | 0 | SBI00589 | Bacillus | axarquiensis | | 1 | 1 | 2 |
| 247 | | bacillus | | | | | | | | | | |
| DMA | SBI04874 | Lentilacto- | buchneri | | 0 | SBI00589 | Bacillus | axarquiensis | | 1 | 1 | 2 |
| 248 | | bacillus | | | | | | | | | | |
| DMA | SBI01292 | Talaromyces | atroroseus | | 0 | SBI04877 | Bacillus | amylolique- | | 2 | 2 | 3 |
| 258 | | | | | | | | faciens | | | | |
| DMA | SBS00319 | Debaryomyces | hansenii | | 0 | SBI00693 | Bacillus | subtilis | | 2 | 2 | 3 |
| 266 | | | | | | | | | | | | |
| DMA | SBI03199 | Candida | dosseyi | | 0 | SBI00693 | Bacillus | subtilis | | 2 | 2 | 3 |
| 267 | | | | | | | | | | | | |
| DMA | SBI05473 | Saccharomyces | cerevisiae | | 0 | SBI00693 | Bacillus | subtilis | | 2 | 2 | 3 |
| 268 | | | | | | | | | | | | |
| DMA | SBI05419 | Kazachstania | servazzii | | 0 | SBI00714 | Bacillus | subtilis | | 2 | 2 | 3 |
| 270 | | | | | | | | | | | | |
| DMA | SBI05449 | Kluyveromyces | marxianus | | 0 | SBI00714 | Bacillus | subtilis | | 2 | 2 | 3 |
| 271 | | | | | | | | | | | | |
| DMA | SBI00264 | Clavispora | sp. | | 0 | SBI00714 | Bacillus | subtilis | | 2 | 2 | 3 |
| 272 | | | | | | | | | | | | |
| DMA | SBS00319 | Debaryomyces | hansenii | | 0 | SBI00714 | Bacillus | subtilis | | 2 | 2 | 3 |
| 273 | | | | | | | | | | | | |
| DMA | SBI04932 | Galactomyces | geotrichum | | 0 | SBI02354 | Bacillus | pumilus | | 1 | 1 | 2 |
| 286 | | | | | | | | | | | | |
| DMA | SBI05419 | Kazachstania | servazzii | | 0 | SBI02354 | Bacillus | pumilus | | 1 | 1 | 2 |
| 287 | | | | | | | | | | | | |
| DMA | SBI05449 | Kluyveromyces | marxianus | | 0 | SBI02354 | Bacillus | pumilus | | 1 | 1 | 2 |
| 288 | | | | | | | | | | | | |
| DMA | SBI00264 | Clavispora | sp. | | 0 | SBI02354 | Bacillus | pumilus | | 1 | 1 | 3 |
| 289 | | | | | | | | | | | | |
| DMA | SBI01681 | Candida | almanticensis | | 0 | SBI00592 | Bacillus | mojavensis | | 2 | 2 | 3 |
| 294 | | | | | | | | | | | | |
| DMA | SBI05469 | Nakazawaea | ishiwadae | | 0 | SBI00592 | Bacillus | mojavensis | | 2 | 2 | 3 |
| 296 | | | | | | | | | | | | |
| DMA | SBI01681 | Candida | almanticensis | | 0 | SBI00693 | Bacillus | subtilis | | 2 | 2 | 3 |
| 297 | | | | | | | | | | | | |
| DMA | SBI05469 | Nakazawaea | ishiwadae | | 0 | SBI00693 | Bacillus | subtilis | | 2 | 2 | 3 |
| 298 | | | | | | | | | | | | |
| DMA | SBI01681 | Candida | almanticensis | | 0 | SBI00714 | Bacillus | subtilis | | 2 | 2 | 3 |
| 299 | | | | | | | | | | | | |
| DMA | SBI05469 | Nakazawaea | ishiwadae | | 0 | SBI00714 | Bacillus | subtilis | | 2 | 2 | 3 |
| 300 | | | | | | | | | | | | |
| DMA | SBI01681 | Candida | almanticensis | | 0) | SBI03602 | Bacillus | subtilis | | 2 | 2 | 3 |
| 302 | | | | | | | | | | | | |
| DMA | SBI05469 | Nakazawaea | ishiwadae | | 0 | SBI03602 | Bacillus | subtilis | | 2 | 2 | 3 |
| 304 | | | | | | | | | | | | |
| DMA | SBI00120 | Holtermanniella | takashimae | | 0 | SBI03602 | Bacillus | subtilis | | 2 | 2 | 3 |
| 305 | | | | | | | | | | | | |
| DMA | SBI01681 | Candida | almanticensis | | 0 | CS00001 | Bacillus | subtilis | DE111 | 1 | 1 | 2 |
| 308 | | | | | | | | | | | | |
| DMA | SBI00264 | Clavispora | sp. | | 0 | SBI03599 | Bacillus | endophyticus | | 2 | 2 | 3 |
| 309 | | | | | | | | | | | | |
| DMA | SBS00319 | Debaryomyces | hansenii | | 0 | SBI03599 | Bacillus | endophyticus | | 2 | 2 | 3 |
| 313 | | | | | | | | | | | | |
| DMA | SBI01681 | Candida | almanticensis | | 0 | SBI05579 | Strepto- | thermophilus | | 0 | 0 | 1 |
| 317 | | | | | | | coccus | | | | | |
| DMA | SBS00319 | Debaryomyces | hansenii | | 0 | SBI05579 | Strepto- | thermophilus | | 0 | 0 | 1 |
| 319 | | | | | | | coccus | | | | | |
| DMA | SBI03199 | Candida | dosseyi | | 0 | SBI05579 | Strepto- | thermophilus | | 0 | 0 | 1 |
| 320 | | | | | | | coccus | | | | | |
| DMA | SBI01681 | Candida | almanticensis | | 0 | CS00008 | Lactobacillus | acidophilus | La-14 | 1 | 1 | 2 |
| 335 | | | | | | | | | | | | |
| DMA | SBS00208 | Pichia | terricola | | 0 | CS00008 | Lactobacillus | acidophilus | La-14 | 1 | 1 | 2 |
| 336 | | | | | | | | | | | | |
| DMA | SBS00319 | Debaryomyces | hansenii | | 0 | CS00008 | Lactobacillus | acidophilus | La-14 | 1 | 1 | 2 |
| 337 | | | | | | | | | | | | |
| DMA | SBI03199 | Candida | dosseyi | | 0 | CS00008 | Lactobacillus | acidophilus | La-14 | 1 | 1 | 2 |
| 338 | | | | | | | | | | | | |
| DMA | SBI04932 | Galactomyces | geotrichum | | 0 | SBI03602 | Bacillus | subtilis | | 2 | 2 | 3 |
| 343 | | | | | | | | | | | | |
| DMA | SBI05419 | Kazachstania | servazzii | | 0 | SBI03602 | Bacillus | subtilis | | 2 | 2 | 3 |
| 348 | | | | | | | | | | | | |
| DMA | SBI05449 | Kluyveromyces | marxianus | | 0 | SBI03602 | Bacillus | subtilis | | 2 | 2 | 3 |
| 349 | | | | | | | | | | | | |
| DMA | SBI00264 | Clavispora | sp. | | 0 | SBI03602 | Bacillus | subtilis | | 2 | 2 | 3 |
| 350 | | | | | | | | | | | | |
| DMA | SBS00319 | Debaryomyces | hansenii | | 0 | SBI03602 | Bacillus | subtilis | | 2 | 2 | 3 |
| 351 | | | | | | | | | | | | |
| DMA | SBI03199 | Candida | dosseyi | | 0 | SBI03602 | Bacillus | subtilis | | 2 | 2 | 3 |
| 352 | | | | | | | | | | | | |
| DMA | SBI00449 | Exiguo- | sp. | | 0 | SBI05473 | Saccharo- | cerevisiae | | 0 | 0 | 1 |
| 360 | | bacterium | | | | | myces | | | | | |
| DMA | SBI01595 | Bacillus | vallismortis | | 2 | SBI0817 | Pichia | membrani- | | 0 | 2 | 3 |
| 371 | | | | | | | | faciens | | | | |
| DMA | SBI01595 | Bacillus | vallismortis | | 2 | SBI04263 | Pichia | kudriavzevii | | 0 | 2 | 4 |
| 372 | | | | | | | | | | | | |
| DMA | SBI01595 | Bacillus | vallismortis | | 2 | SBI05473 | Saccharo- | cerevisiae | | 0 | 2 | 3 |
| 373 | | | | | | | myces | | | | | |
| DMA | SBI04011 | Bacillus | megaterium | | 2 | SBI05473 | Saccharo- | cerevisiae | | 0 | 2 | 3 |
| 376 | | | | | | | myces | | | | | |

TABLE 7
Acetate Additivity and Synergy in DMAs

| | | | | | Microbe | | | | | Microbe | Predic- | Combin- |
|---|---|---|---|---|---|---|---|---|---|---|---|---|
| | | | | | #1 | | | | | #2 | ted | ation |
| DMA | Microbe | | | Strain | Acetate | Microbe | | | Strain | Acetate | Acetate | Acetate |
| # | #1 | Genus | Species | ID | (uM) | #2 | Genus | Species | ID | (uM) | (uM) | (uM) |
| DMA | SBI04887 | Lacticasei- | casei | | 0 | SBI00817 | Pichia | fermentans | | 0 | 0 | 567 |
| 37 | | bacillus | | | | | | | | | | |
| DMA | SBI04887 | Lacticasei- | casei | | 0 | SBI01683 | Pichia | fermentans | | 0 | 0 | 376 |
| 40 | | bacillus | | | | | | | | | | |
| DMA | SBI04887 | Lacticasei- | casei | | 0 | SBS04263 | Pichia | kudriavzevii | | 0 | 0 | 679 |
| 43 | | bacillus | | | | | | | | | | |
| DMA | SBI04256 | Lacticasei- | casei | | 530 | SBI00550 | Bacillus | atrophaeus | | 112 | 642 | 1542 |
| 46 | | bacillus | | | | | | | | | | |
| DMA | SBI04256 | Lacticasei- | casei | | 530 | SBI02185 | Bacillus | licheniformis | | 92 | 622 | 799 |
| 50 | | bacillus | | | | | | | | | | |
| DMA | SBI04889 | Lactiplanti- | plantarum | | 646 | SBI02185 | Bacillus | licheniformis | | 92 | 738 | 852 |
| 52 | | bacillus | | | | | | | | | | |
| DMA | SBI00550 | Bacillus | atrophaeus | | 1355 | SBI00817 | Pichia | membranifaciens | | 0 | 1355 | 3587 |
| 56 | | | | | | | | | | | | |
| DMA | SBI02185 | Bacillus | licheniformis | | 4193 | SBI01683 | Pichia | fermentans | | 107 | 4300 | 4746 |
| 58 | | | | | | | | | | | | |
| DMA | SBI02185 | Bacillus | licheniformis | | 4193 | SBS04263 | Pichia | kudriavzevii | | 0 | 4193 | 4390 |
| 61 | | | | | | | | | | | | |
| DMA | SBI01819 | Bacillus | pumilus | | 70 | SBI04910 | Pediococcus | ethanolidurans | | 346 | 416 | 4313 |
| 64 | | | | | | | | | | | | |
| DMA | SBI00550 | Bacillus | atrophaeus | | 1355 | SBI04913 | Schleiferi- | harbinensis | | 715 | 2070 | 7802 |
| 69 | | | | | | | lactobacillus | | | | | |
| DMA | SBI03005 | Bacillus | altitudinis | | 1451 | SBI04918 | Lactococcus | lactis | | 7712 | 9162 | 10315 |
| 77 | | | | | | | | | | | | |
| DMA | SBI00550 | Bacillus | atrophaeus | | 1355 | SBI05697 | Lactiplanti- | plantarum | | 8944 | 10299 | 12112 |
| 78 | | | | | | | bacillus | | | | | |
| DMA | SBI00997 | Bacillus | safensis | | 1239 | SBI05697 | Lactiplanti- | plantarum | | 8944 | 10183 | 10225 |
| 79 | | | | | | | bacillus | | | | | |
| DMA | SBI01819 | Bacillus | pumilus | | 70 | SBI05697 | Lactiplanti- | plantarum | | 8944 | 9014 | 12172 |
| 80 | | | | | | | bacillus | | | | | |
| DMA | SBI02185 | Bacillus | licheniformis | | 4193 | SBI05697 | Lactiplanti- | plantarum | | 8944 | 13137 | 15836 |
| 81 | | | | | | | bacillus | | | | | |
| DMA | SBI04173 | Bacillus | paralicheni- | | 3996 | SBI05697 | Lactiplanti- | plantarum | | 8944 | 12940 | 15202 |
| 82 | | | formis | | | | bacillus | | | | | |
| DMA | SBI03005 | Bacillus | altitudinis | | 1451 | SBI05697 | Lactiplanti- | plantarum | | 8944 | 10395 | 11353 |
| 83 | | | | | | | bacillus | | | | | |
| DMA | SBI04877 | Bacillus | amylolique- | | 965 | SBI05697 | Lactiplanti- | plantarum | | 8944 | 9909 | 11583 |
| 84 | | | faciens | | | | bacillus | | | | | |
| DMA | SBI04874 | Lentilacto- | buchneri | | 463 | CS00018 | Saccharo- | boulardii | | 51 | 514 | 927 |
| 85 | | bacillus | | | | | myces | | | | | |
| DMA | SBI04874 | Lentilacto- | buchneri | | 463 | SBI05419 | Kazachstania | servazzii | | 41 | 504 | 551 |
| 86 | | bacillus | | | | | | | | | | |
| DMA | SBI04874 | Lentilacto- | buchneri | | 463 | SBI05449 | Kluyvero- | marxianus | | 0 | 463 | 780 |
| 87 | | bacillus | | | | | myces | | | | | |
| DMA | SBI04874 | Lentilacto- | buchneri | | 463 | SBI00553 | Bacillus | subtilis | | 285 | 748 | 848 |
| 88 | | bacillus | | | | | | | | | | |
| DMA | SBI04874 | Lentilacto- | buchneri | | 463 | SBI00449 | Exiguo- | sp. | | 2600 | 3063 | 3980 |
| 89 | | bacillus | | | | | bacterium | | | | | |
| DMA | SBI04874 | Lentilacto- | buchneri | | 463 | SBI00556 | Paenibacillus | sp. | | 5524 | 5987 | 6739 |
| 90 | | bacillus | | | | | | | | | | |
| DMA | SBI04874 | Lentilacto- | buchneri | | 463 | SBI00564 | Bacillus | subtilis | | 459 | 921 | 1088 |
| 91 | | bacillus | | | | | | | | | | |
| DMA | SBI04873 | Pediococcus | ethanoli- | | 34 | CS00018 | Saccharo- | boulardii | | 51 | 84 | 158 |
| 92 | | | durans | | | | myces | | | | | |
| DMA | SBI04873 | Pediococcus | ethanoli- | | 34 | SBI05449 | Kluyvero- | marxianus | | 0 | 34 | 276 |
| 93 | | | durans | | | | myces | | | | | |
| DMA | SBI04873 | Pediococcus | ethanoli- | | 34 | SBI00553 | Bacillus | subtilis | | 285 | 319 | 687 |
| 94 | | | durans | | | | | | | | | |
| DMA | SBI04873 | Pediococcus | ethanoli- | | 34 | SBI00564 | Bacillus | subtilis | | 459 | 492 | 628 |
| 97 | | | durans | | | | | | | | | |
| DMA | CS00010 | Lacticasei- | rhamnosus | Lr-32 | 2358 | CS00018 | Saccharo- | boulardii | | 0 | 2358 | 2983 |
| 98 | | bacillus | | | | | myces | | | | | |
| DMA | SBI04902 | Pediococcus | pentosaceus | | 759 | SBI00553 | Bacillus | subtilis | | 285 | 1045 | 6130 |
| 100 | | | | | | | | | | | | |
| DMA | SBI04910 | Pediococcus | ethanoli- | | 97 | CS00018 | Saccharo- | boulardii | | 51 | 148 | 429 |
| 101 | | | durans | | | | myces | | | | | |
| DMA | SBI04910 | Pediococcus | ethanoli- | | 97 | SBI05419 | Kazachstania | servazzii | | 41 | 138 | 327 |
| 102 | | | durans | | | | | | | | | |
| DMA | SBI04910 | Pediococcus | ethanoli- | | 97 | SBI05449 | Kluyvero- | marxianus | | 0 | 97 | 765 |
| 103 | | | durans | | | | myces | | | | | |
| DMA | SBI04910 | Pediococcus | ethanoli- | | 97 | SBI00553 | Bacillus | subtilis | | 285 | 382 | 568 |
| 104 | | | durans | | | | | | | | | |
| DMA | SBI04910 | Pediococcus | ethanoli- | | 97 | SBI00564 | Bacillus | subtilis | | 459 | 556 | 819 |
| 107 | | | durans | | | | | | | | | |
| DMA | SBI05697 | Lactiplanti- | plantarum | | 3372 | CS00018 | Saccharo- | boulardii | | 51 | 3423 | 4928 |
| 114 | | bacillus | | | | | myces | | | | | |
| DMA | SBI05697 | Lactiplanti- | plantarum | | 3372 | SBI01679 | Candida | sp. | | 0 | 3372 | 4949 |
| 115 | | bacillus | | | | | | | | | | |
| DMA | SBI05697 | Lactiplanti- | plantarum | | 3372 | SBI05419 | Kazachstania | servazzii | | 41 | 3413 | 3849 |
| 116 | | bacillus | | | | | | | | | | |
| DMA | SBI05697 | Lactiplanti- | plantarum | | 3372 | SBI05449 | Kluyvero- | marxianus | | 0 | 3372 | 5386 |
| 117 | | bacillus | | | | | myces | | | | | |
| DMA | SBI05697 | Lactiplanti- | plantarum | | 3372 | SBI05469 | Nakazawaea | ishiwadae | | 0 | 3372 | 3588 |
| 118 | | bacillus | | | | | | | | | | |
| DMA | SBI05697 | Lactiplanti- | plantarum | | 3372 | SBI00553 | Bacillus | subtilis | | 285 | 3657 | 5233 |
| 119 | | bacillus | | | | | | | | | | |
| DMA | SBI05697 | Lactiplanti- | plantarum | | 3372 | SBI00449 | Exiguo- | sp. | | 2600 | 5972 | 7881 |
| 120 | | bacillus | | | | | bacterium | | | | | |
| DMA | SBI05697 | Lactiplanti- | plantarum | | 3372 | SBI00556 | Paenibacillus | sp. | | 5524 | 8896 | 9760 |
| 121 | | bacillus | | | | | | | | | | |
| DMA | SBI05697 | Lactiplanti- | plantarum | | 3372 | SBI00564 | Bacillus | subtilis | | 459 | 3831 | 4804 |
| 122 | | bacillus | | | | | | | | | | |
| DMA | SBI05697 | Lactiplanti- | plantarum | | 3372 | SBI00568 | Psychro- | sp. | | 339 | 3711 | 4331 |
| 123 | | bacillus | | | | | bacillus | | | | | |
| DMA | SBI00553 | Bacillus | subtilis | | 611 | SBI05399 | Lactiplanti- | plantarum | | 5245 | 5856 | 6139 |
| 125 | | | | | | | bacillus | | | | | |
| DMA | SBI00449 | Exiguo- | sp. | | 5290 | SBS04255 | Leuconostoc | mesenteroides | | 1328 | 6618 | 8575 |
| 131 | | bacterium | | | | | | | | | | |
| DMA | SBI00556 | Paeni- | sp. | | 10294 | CS00018 | Saccharo- | boulardii | | 1210 | 11504 | 11498 |
| 134 | | bacillus | | | | | myces | | | | | |
| DMA | SBI00556 | Paeni- | sp. | | 10294 | SBI04881 | Lentilacto- | buchneri | | 911 | 11205 | 13851 |
| 137 | | bacillus | | | | | bacillus | | | | | |
| DMA | SBI00564 | Bacillus | subtilis | | 444 | SBI04900 | Leuconostoc | pseudo- | | 1933 | 2377 | 4783 |
| 143 | | | | | | | | mesenteroides | | | | |
| DMA | SBI00564 | Bacillus | subtilis | | 444 | SBI05399 | Lactiplanti- | plantarum | | 5245 | 5689 | 6499 |
| 379 | | | | | | | bacillus | | | | | |
| DMA | SBI00568 | Psychro- | sp. | | 59 | SBI05399 | Lactiplanti- | plantarum | | 5245 | 5304 | 5992 |
| 145 | | bacillus | | | | | bacillus | | | | | |
| DMA | SBI01595 | Bacillus | amylolique- | | 229 | CS00018 | Saccharo- | boulardii | | 0 | 229 | 593 |
| 146 | | | faciens | | | | myces | | | | | |
| DMA | SBI01595 | Bacillus | amylolique- | | 222 | SBI05399 | Lactiplanti- | plantarum | | 5245 | 5467 | 5599 |
| 154 | | | faciens | | | | bacillus | | | | | |
| DMA | SBI01187 | Pichia | membrani- | | 0 | SBI04006 | Bacillus | subtilis | | 0 | 0 | 637 |
| 179 | | | faciens | | | | | | | | | |
| DMA | SBI01679 | Candida | sp. | | 0 | SBI04006 | Bacillus | subtilis | | 0 | 0 | 977 |
| 180 | | | | | | | | | | | | |
| DMA | SBI01187 | Pichia | membrani- | | 0 | CS00001 | Bacillus | subtilis | DE111 | 4320 | 4320 | 5435 |
| 181 | | | faciens | | | | | | | | | |
| DMA | SBI01679 | Candida | sp. | | 0 | SBS04260 | Lactiplanti- | plantarum | | 3549 | 3549 | 3696 |
| 183 | | | | | | | bacillus | | | | | |
| DMA | SBI04916 | Lactococcus | lactis | | 6467 | SBI00120 | Holter- | takashimae | | 0 | 6467 | 6944 |
| 196 | | | | | | | manniella | | | | | |
| DMA | SBS04260 | Lactiplanti- | plantarum | | 1819 | SBI00120 | Holter- | takashimae | | 0 | 1819 | 2904 |
| 197 | | bacillus | | | | | manniella | | | | | |
| DMA | SBI01292 | Talaromyces | atroroseus | | 0 | SBI04918 | Lactococcus | lactis | | 6170 | 6170 | 6293 |
| 198 | | | | | | | | | | | | |
| DMA | SBI01292 | Talaromyces | atroroseus | | 0 | SBS04260 | Lactiplanti- | plantarum | | 3549 | 3549 | 4854 |
| 199 | | | | | | | bacillus | | | | | |
| DMA | SBS04260 | Lactiplanti- | plantarum | | 1819 | SBI03599 | Bacillus | endophyticus | | 10 | 1829 | 2169 |
| 201 | | bacillus | | | | | | | | | | |
| DMA | SBS04260 | Lactiplanti- | plantarum | | 1819 | SBI00592 | Bacillus | mojavensis | | 1009 | 2828 | 3391 |
| 202 | | bacillus | | | | | | | | | | |
| DMA | SBS04260 | Lactiplanti- | plantarum | | 1819 | SBI00693 | Bacillus | subtilis | | 1212 | 3032 | 3571 |
| 203 | | bacillus | | | | | | | | | | |
| DMA | SBS04260 | Lactiplanti- | plantarum | | 1819 | SBI00714 | Bacillus | subtilis | | 1680 | 3500 | 3883 |
| 204 | | bacillus | | | | | | | | | | |
| DMA | SBS04260 | Lactiplanti- | plantarum | | 1819 | SBI02181 | Bacillus | amyloliquefaciens | | 342 | 2161 | 2533 |
| 205 | | bacillus | | | | | | | | | | |
| DMA | SBS04260 | Lactiplanti- | plantarum | | 1819 | SBI04006 | Bacillus | subtilis | | 1704 | 3523 | 3608 |
| 206 | | bacillus | | | | | | | | | | |
| DMA | SBI01292 | Talaromyces | atroroseus | | 0 | SBI00714 | Bacillus | subtilis | | 1408 | 1408 | 2354 |
| 216 | | | | | | | | | | | | |
| DMA | SBI01292 | Talaromyces | atroroseus | | 0 | SBI04006 | Bacillus | subtilis | | 609 | 609 | 911 |
| 217 | | | | | | | | | | | | |
| DMA | SBI00714 | Bacillus | subtilis | | 842 | SBI04256 | Lacticasei- | casei | | 938 | 1779 | 1923 |
| 220 | | | | | | | bacillus | | | | | |
| DMA | SBI03599 | Bacillus | endophyticus | | 205 | SBI04887 | Lacticasei- | casei | | 467 | 672 | 667 |
| 230 | | | | | | | bacillus | | | | | |
| DMA | SBI00592 | Bacillus | mojavensis | | 896 | SBI04887 | Lacticasei- | casei | | 467 | 1363 | 1436 |
| 231 | | | | | | | bacillus | | | | | |
| DMA | SBI00693 | Bacillus | subtilis | | 881 | SBI04887 | Lacticasei- | casei | | 467 | 1348 | 1454 |
| 232 | | | | | | | bacillus | | | | | |
| DMA | SBI00714 | Bacillus | subtilis | | 842 | SBI04887 | Lacticasei- | casei | | 467 | 1309 | 1569 |
| 233 | | | | | | | bacillus | | | | | |
| DMA | SBI00461 | Bacillus | safensis | | 912 | SBI04887 | Lacticasei- | casei | | 467 | 1379 | 1591 |
| 237 | | | | | | | bacillus | | | | | |
| DMA | SBI04874 | Lentilacto- | buchneri | | 938 | SBI00225 | Clavispora | sp. | | 0 | 938 | 1041 |
| 238 | | bacillus | | | | | | | | | | |
| DMA | CS00008 | Lacto- | acidophilus | La-14 | 451 | SBI01292 | Talaromyces | atroroseus | | 0 | 451 | 670 |
| 239 | | bacillus | | | | | | | | | | |
| DMA | CS00010 | Lacticasei- | rhamnosus | Lr-32 | 2358 | SBI01292 | Talaromyces | atroroseus | | 0 | 2358 | 3124 |
| 241 | | bacillus | | | | | | | | | | |
| DMA | SBS04254 | Levilacto- | brevis | | 1198 | CS00018 | Saccharo- | boulardii | | 0 | 1198 | 1372 |
| 243 | | bacillus | | | | | myces | | | | | |
| DMA | SBI05579 | Strepto- | thermo- | | 5194 | SBI03599 | Bacillus | endophytic | | 299 | 5493 | 5642 |
| 244 | | coccus | philus | | | | | us | | | | |
| DMA | SBI00598 | Bacillus | subtilis | | 1853 | SBI04874 | Lentilacto- | buchneri | | 1978 | 3831 | 3848 |
| 249 | | | | | | | bacillus | | | | | |
| DMA | CS00008 | Lacto- | acidophilus | La-14 | 451 | SBI01595 | Bacillus | amyloliquefaciens | | 0 | 451 | 618 |
| 250 | | bacillus | | | | | | | | | | |
| DMA | CS00008 | Lacto- | acidophilus | La-14 | 451 | SBI03602 | Bacillus | subtilis | | 591 | 1042 | 1251 |
| 252 | | bacillus | | | | | | | | | | |
| DMA | CS00008 | Lacto- | acidophilus | La-14 | 451 | SBI04877 | Bacillus | amyloliquefaciens | | 197 | 648 | 1114 |
| 253 | | bacillus | | | | | | | | | | |
| DMA | CS00007 | Lactiplanti- | plantarum | 299V | 6247 | SBI00461 | Bacillus | safensis | | 373 | 6621 | 6724 |
| 255 | | bacillus | | | | | | | | | | |
| DMA | SBI01292 | Talaromyces | atroroseus | | 0 | CS00001 | Bacillus | subtilis | DE111 | 4320 | 4320 | 5064 |
| 256 | | | | | | | | | | | | |
| DMA | SBI04877 | Bacillus | amylolique- | | 327 | SBI01292 | Talaromyces | atroroseus | | 0 | 327 | 460 |
| 258 | | | faciens | | | | | | | | | |
| DMA | CS00001 | Bacillus | subtilis | DE111 | 1927 | CS00018 | Saccharo- | boulardii | | 0 | 1927 | 2611 |
| 260 | | | | | | | myces | | | | | |
| DMA | SBI00589 | Bacillus | axarquiensis | | 2056 | CS00018 | Saccharo- | boulardii | | 0 | 2056 | 2519 |
| 261 | | | | | | | myces | | | | | |
| DMA | SBI00598 | Bacillus | subtilis | | 1853 | CS00018 | Saccharo- | boulardii | | 0 | 1853 | 2090 |
| 262 | | | | | | | myces | | | | | |
| DMA | SBI04877 | Bacillus | amylolique- | | 327 | CS00018 | Saccharo- | boulardii | | 0 | 327 | 994 |
| 263 | | | faciens | | | | myces | | | | | |
| DMA | SBI00644 | Bacillus | aryabhattai | | 1325 | CS00018 | Saccharo- | boulardii | | 0 | 1325 | 1982 |
| 264 | | | | | | | myces | | | | | |
| DMA | SBI05473 | Saccharo- | cerevisiae | | 711 | SBI04916 | Lactococcus | lactis | | 1280 | 1991 | 2491 |
| 265 | | myces | | | | | | | | | | |
| DMA | SBI03199 | Candida | dosseyi | | 911 | SBI00714 | Bacillus | subtilis | | 1823 | 2734 | 3128 |
| 274 | | | | | | | | | | | | |
| DMA | CS00001 | Bacillus | subtilis | DE111 | 1927 | SBI04873 | Pediococcus | ethanolidurans | | 0 | 1927 | 2403 |
| 275 | | | | | | | | | | | | |
| DMA | SBI00598 | Bacillus | subtilis | | 1853 | SBI04873 | Pediococcus | ethanolidurans | | 0) | 1853 | 3356 |
| 277 | | | | | | | | | | | | |
| DMA | SBI03199 | Candida | dosseyi | | 911 | SBI02181 | Bacillus | amyloliquefaciens | | 487 | 1398 | 4331 |
| 283 | | | | | | | | | | | | |
| DMA | SBI05473 | Saccharo- | cerevisiae | | 420 | SBI02181 | Bacillus | amyloliquefaciens | | 487 | 906 | 2599 |
| 284 | | myces | | | | | | | | | | |
| DMA | SBI00644 | Bacillus | aryabhattai | | 1325 | SBI04874 | Lentilacto- | buchneri | | 1978 | 3303 | 3864 |
| 285 | | | | | | | bacillus | | | | | |
| DMA | SBS00319 | Debaryo- | hansenii | | 0 | SBI02354 | Bacillus | pumilus | | 3702 | 3702 | 5026 |
| 290 | | myces | | | | | | | | | | |
| DMA | SBI03199 | Candida | dosseyi | | 911 | SBI02354 | Bacillus | pumilus | | 3702 | 4613 | 5294 |
| 291 | | | | | | | | | | | | |
| DMA | SBI01681 | Candida | akabanensis | | 258 | SBI05579 | Strepto- | thermophilus | | 6720 | 6978 | 7254 |
| 317 | | | | | | | coccus | | | | | |
| DMA | SBS00319 | Debaryo- | hansenii | | 300 | SBI05579 | Strepto- | thermophilus | | 6720 | 7020 | 7169 |
| 319 | | myces | | | | | coccus | | | | | |
| DMA | SBI03199 | Candida | dosseyi | | 364 | SBI05579 | Strepto- | thermophilus | | 6720 | 7083 | 7090 |
| 320 | | | | | | | coccus | | | | | |
| DMA | SBI01681 | Candida | akabanensis | | 258 | CS00007 | Lactiplanti | plantarum | 299V | 6417 | 6674 | 7425 |
| 322 | | | | | | | bacillus | | | | | |
| DMA | SBS00208 | Pichia | terricola | | −125 | CS00007 | Lactiplanti | plantarum | 299V | 6417 | 6292 | 8121 |
| 323 | | | | | | | bacillus | | | | | |
| DMA | SBS00319 | Debaryo- | hansenii | | 300 | CS00007 | Lactiplanti | plantarum | 299V | 6417 | 6717 | 6961 |
| 324 | | myces | | | | | bacillus | | | | | |
| DMA | SBI03199 | Candida | dosseyi | | 364 | CS00007 | Lactiplanti | plantarum | 299V | 6417 | 6780 | 7442 |
| 325 | | | | | | | bacillus | | | | | |
| DMA | SBI05473 | Saccharo- | cerevisiae | | 711 | SBI04918 | Lactococcus | lactis | | 4099 | 4810 | 5184 |
| 326 | | myces | | | | | | | | | | |
| DMA | SBI01681 | Candida | akabanensis | | 258 | SBI04889 | Lactiplanti- | plantarum | | 5799 | 6057 | 6183 |
| 328 | | | | | | | bacillus | | | | | |
| DMA | SBI03199 | Candida | dosseyi | | 364 | SBI04889 | Lactiplanti- | plantarum | | 5799 | 6163 | 6778 |
| 329 | | | | | | | bacillus | | | | | |
| DMA | SBI05473 | Saccharo- | cerevisiae | | 711 | SBI04895 | Lacticasei- | casei | | 4065 | 4776 | 5186 |
| 330 | | myces | | | | | bacillus | | | | | |
| DMA | SBI01681 | Candida | akabanensis | | 258 | SBI04895 | Lacticasei- | casei | | 4065 | 4323 | 4414 |
| 331 | | | | | | | bacillus | | | | | |
| DMA | SBI03199 | Candida | dosseyi | | 364 | SBI04895 | Lacticasei- | casei | | 4065 | 4429 | 4501 |
| 332 | | | | | | | bacillus | | | | | |
| DMA | SBI01681 | Candida | akabanensis | | 258 | SBS04260 | Lactiplanti- | plantarum | | 5767 | 6025 | 6569 |
| 334 | | | | | | | bacillus | | | | | |
| DMA | SBI01681 | Candida | akabanensis | | 258 | SBI04916 | Lactococcus | lactis | | 1280 | 1538 | 2198 |
| 339 | | | | | | | | | | | | |
| DMA | SBI03199 | Candida | dosseyi | | 364 | SBI04916 | Lactococcus | lactis | | 1280 | 1644 | 1813 |
| 340 | | | | | | | | | | | | |
| DMA | SBI05473 | Saccharo- | cerevisiae | | 711 | SBI00598 | Bacillus | subtilis | | 729 | 1440 | 1495 |
| 341 | | myces | | | | | | | | | | |
| DMA | SBI01681 | Candida | akabanensis | | 258 | SBI00598 | Bacillus | subtilis | | 729 | 987 | 1044 |
| 342 | | | | | | | | | | | | |
| DMA | SBS00319 | Debaryo- | hansenii | | 300 | SBI00598 | Bacillus | subtilis | | 729 | 1029 | 7426 |
| 344 | | myces | | | | | | | | | | |
| DMA | SBI03199 | Candida | dosseyi | | 364 | SBI00598 | Bacillus | subtilis | | 729 | 1093 | 1510 |
| 345 | | | | | | | | | | | | |
| DMA | SBI05473 | Saccharo- | cerevisiae | | 711 | SBI00644 | Bacillus | aryabhattai | | 501 | 1212 | 1281 |
| 346 | | myces | | | | | | | | | | |
| DMA | SBI05419 | Kazachstania | servazzii | | 1098 | SBI03602 | Bacillus | subtilis | | 1744 | 2842 | 2849 |
| 348 | | | | | | | | | | | | |
| DMA | SBI03199 | Candida | dosseyi | | 911 | SBI04006 | Bacillus | subtilis | | 3965 | 4877 | 5854 |
| 354 | | | | | | | | | | | | |
| DMA | SBI03199 | Candida | dosseyi | | 911 | CS00001 | Bacillus | subtilis | DE111 | 5950 | 6861 | 6960 |
| 357 | | | | | | | | | | | | |
| DMA | SBI00449 | Exiguo- | sp. | | 4296 | SBI05473 | Saccharo- | cerevisiae | | 549 | 4845 | 4979 |
| 360 | | bacterium | | | | | myces | | | | | |
| DMA | CS00017 | Lacticasei- | paracasei | Shirota | 3128 | SBI00564 | Bacillus | subtilis | | 252 | 3380 | 3388 |
| 361 | | bacillus | | | | | | | | | | |
| DMA | CS00017 | Lacticasei- | paracasei | Shirota | 3128 | SBI01595 | Bacillus | amyloliquefaciens | | 0 | 3128 | 3229 |
| 362 | | bacillus | | | | | | | | | | |
| DMA | CS00017 | Lacticasei- | paracasei | Shirota | 3128 | SBI05449 | Kluyvero- | marxianus | | 401 | 3528 | 3800 |
| 364 | | bacillus | | | | | myces | | | | | |
| DMA | CS00017 | Lacticasei- | paracasei | Shirota | 3128 | CS00018 | Saccharo- | boulardii | | 703 | 3830 | 4281 |
| 366 | | bacillus | | | | | myces | | | | | |
| DMA | SBI00553 | Bacillus | subtilis | | 75 | SBI00817 | Pichia | fermentans | | 254 | 329 | 399 |
| 369 | | | | | | | | | | | | |
| DMA | SBI01595 | Bacillus | amylolique- | | 0 | SBI00817 | Pichia | fermentans | | 254 | 254 | 418 |
| 371 | | | faciens | | | | | | | | | |
| DMA | SBI04011 | Bacillus | megaterium | | 0 | SBI00817 | Pichia | fermentans | | 254 | 254 | 299 |
| 375 | | | | | | | | | | | | |
| DMA | CS00020 | Weizmannia | coagulans | BC30 | 1261 | SBI00817 | Pichia | fermentans | | 254 | 1515 | 1752 |
| 377 | | | | | | | | | | | | |
| DMA | CS00020 | Weizmannia | coagulans | BC30 | 1261 | SBI05473 | Saccharo- | cerevisiae | | 549 | 1810 | 2215 |
| 378 | | | | | | | myces | | | | | |
| DMA | SBI05579 | Strepto- | thermophilus | | 5194 | SBI03602 | Bacillus | subtilis | | 462 | 5657 | 8004 |
| 380 | | coccus | | | | | | | | | | |
| DMA | SBI04259 | Weisella | cibaria | | 3105 | SBI03602 | Bacillus | subtilis | | 462 | 3567 | 4718 |
| 381 | | | | | | | | | | | | |

TABLE 8
Propionate Additivity and Synergy in DMAs

| | | | | | Microbe | | | | | Microbe | Predicted | |
|---|---|---|---|---|---|---|---|---|---|---|---|---|
| | | | | | #1 | | | | | #2 | Combi- | Actual |
| | | | | | Prop- | | | | | Propi- | nation | Propi- |
| DMA | Microbe | | | Strain | ionate | Microbe | | | Strain | onate | Propionate | onate |
| # | #1 | Genus | Species | ID | (uM) | #2 | Genus | Species | ID | (uM) | (uM) | (uM) |
| DMA | SBI00550 | Bacillus | atrophae | | 101 | SBI00817 | Pichia | fermentans | | 0 | 101 | 459 |
| 56 | | | | | | | | | | | | |
| DMA | SBI04173 | Bacillus | paralicheni- | | 304 | SBI01187 | Pichia | membranifaciens | | 0 | 304 | 422 |
| 57 | | | formis | | | | | | | | | |
| DMA | SBI04173 | Bacillus | paralicheni- | | 304 | SBI02945 | Meyerozyma | guilliermondii | | 0 | 304 | 38 |
| 59 | | | formis | | | | | | | | | |
| DMA | SBI04173 | Bacillus | paralicheni- | | 304 | SBS04263 | Pichia | kudriavzevii | | 0 | 304 | 320 |
| 62 | | | formis | | | | | | | | | |
| DMA | SBI01819 | Bacillus | pumilus | | 0 | SBI04910 | Pediococcus | ethanolidurans | | 0 | 0 | 518 |
| 64 | | | | | | | | | | | | |
| DMA | SBI00553 | Bacillus | subtilis | | 0 | SBI04902 | Pediococcus | pentosaceus | | 0 | 0 | 364 |
| 100 | | | | | | | | | | | | |
| DMA | SBI00556 | Paeni- | sp. | | 16 | SBI05449 | Kluyveros- | marxianus | | 0 | 16 | 262 |
| 135 | | bacillus | | | | | myces | | | | | |
| DMA | SBI01595 | Bacillus | amylolique- | | 50 | CS00018 | Saccharo- | boulardii | | 0 | 50 | 447 |
| 146 | | | faciens | | | | myces | | | | | |
| DMA | SBI03602 | Bacillus | subtilis | | 67 | SBI01187 | Pichia | membranifaciens | | 295 | 361 | 1157 |
| 178 | | | | | | | | | | | | |
| DMA | SBI04006 | Bacillus | subtilis | DE111 | 0 | SBI01187 | Pichia | membranifaciens | | 295 | 295 | 2469 |
| 179 | | | | | | | | | | | | |
| DMA | CS00001 | Bacillus | subtilis | DE111 | 0 | SBI01187 | Pichia | membranifaciens | | 295 | 295 | 4893 |
| 181 | | | | | | | | | | | | |
| DMA | SBI00592 | Bacillus | mojavensis | | 0 | SBI00120 | Kwoniella | shandongens | | 0 | 0 | 270 |
| 207 | | | | | | | | | | | | |
| DMA | SBI00714 | Bacillus | subtilis | | 0 | SBI00120 | Kwoniella | shandongens | | 0 | 0 | 280 |
| 209 | | | | | | | | | | | | |
| DMA | SBI00693 | Bacillus | subtilis | | 233 | SBI04256 | Lacticasei- | casei | | 303 | 536 | 580 |
| 219 | | | | | | | bacillus | | | | | |
| DMA | SBI00714 | Bacillus | subtilis | | 0 | SBI04256 | Lacticasei- | casei | | 303 | 303 | 622 |
| 220 | | | | | | | bacillus | | | | | |
| DMA | SBI04006 | Bacillus | subtilis | | 108 | SBI04256 | Lacticasei- | casei | | 303 | 411 | 518 |
| 221 | | | | | | | bacillus | | | | | |
| DMA | SBI00461 | Bacillus | safensis | | 295 | SBI04256 | Lacticasei- | casei | | 303 | 598 | 765 |
| 222 | | | | | | | bacillus | | | | | |
| DMA | SBI00714 | Bacillus | subtilis | | 0 | SBI04881 | Lentilacto- | buchneri | | 447 | 447 | 596 |
| 225 | | | | | | | bacillus | | | | | |
| DMA | SBI04006 | Bacillus | subtilis | | 108 | SBI04881 | Lentilacto- | buchneri | | 447 | 555 | 621 |
| 226 | | | | | | | bacillus | | | | | |
| DMA | SBI04006 | Bacillus | subtilis | | 108 | SBI04884 | Enterococcus | gilvus | | 1616 | 1724 | 1741 |
| 228 | | | | | | | | | | | | |
| DMA | SBI03599 | Bacillus | endophyticus | | 66 | SBI04887 | Lacticasei- | casei | | 151 | 217 | 216 |
| 230 | | | | | | | bacillus | | | | | |
| DMA | SBI00592 | Bacillus | mojavensis | | 313 | SBI04887 | Lacticasei- | casei | | 151 | 464 | 464 |
| 231 | | | | | | | bacillus | | | | | |
| DMA | SBI00693 | Bacillus | subtilis | | 233 | SBI04887 | Lacticasei- | casei | | 151 | 384 | 470 |
| 232 | | | | | | | bacillus | | | | | |
| DMA | SBI00714 | Bacillus | subtilis | | 0 | SBI04887 | Lacticasei- | casei | | 151 | 151 | 507 |
| 233 | | | | | | | bacillus | | | | | |
| DMA | SBI04006 | Bacillus | subtilis | | 108 | SBI04887 | Lacticasei- | casei | | 151 | 259 | 451 |
| 236 | | | | | | | bacillus | | | | | |
| DMA | SBI00461 | Bacillus | safensis | | 295 | SBI04887 | Lacticasei- | casei | | 151 | 446 | 514 |
| 237 | | | | | | | bacillus | | | | | |
| DMA | SBI01595 | Bacillus | amylolique- | | 268 | SBI04874 | Lentilacto- | buchneri | | 0 | 268 | 440 |
| 251 | | | faciens | | | | bacillus | | | | | |
| DMA | SBI04877 | Bacillus | amylolique- | | 222 | CS00008 | Lacto- | acidophilus | La-14 | 0 | 222 | 276 |
| 253 | | | faciens | | | | bacillus | | | | | |
| DMA | SBI04877 | Bacillus | amylolique- | | 222 | SBI04874 | Lentilacto- | buchneri | | 0 | 222 | 428 |
| 254 | | | faciens | | | | bacillus | | | | | |
| DMA | SBI01595 | Bacillus | amylolique- | | 427 | SBI04873 | Pediococcus | ethanolidurans | | 0 | 427 | 452 |
| 278 | | | faciens | | | | | | | | | |
| DMA | SBI00598 | Bacillus | subtilis | | 88 | SBS00319 | Debaryo- | hansenii | | 0 | 88 | 355 |
| 344 | | | | | | | myces | | | | | |

### Example 9: In Vitro Testing of DMAs for Effects on Human Cells

Following the in silico identification of DMAs with therapeutic potential, the desired phenotypes are confirmed experimentally using human cell culture techniques. Individual microbes are grown in pure culture and then combined at equal proportions to test the effects of the resulting combination (DMA) on human cells utilizing several culture-based assays.

To empirically test the immunomodulatory capacity of DMAs in vitro, mammalian immune cells are treated with microbially conditioned supernatant. Human U937 cells are myelocyte lineage cells which can be differentiated into macrophage-like cells with ionophores, such as phorbol 12-myristate 13-acetate (PMA). PMA differentiated U937 macrophages are incubated with microbially conditioned supernatant. The subsequent immune response is analyzed by enzyme-linked immunosorbent assay (ELISA) and/or qRT-PCR to quantify the specific cytokines including but not limited to TNF-α, IL-1β, IL-2, IL-3, IL-4, IL-5, IL-6, IL-7, IL-8, IL-9, IL-10, IL-12, IL-13, IL-15, IL-17, IL-21, IL-22, IL-23, IL-27 IFNγ, G-CSF, GM-CSF, IP-10, CXCL-1, CXCL-2, LIF, LIX, MCP-1 that are produced in response to the microbial supernatant. Through examination of the pro- and anti-inflammatory cytokines that are produced by these immune cells, the effect of the microbes of interest on the immune system is discerned.

Specific immunomodulatory pathways are also examined. The aryl hydrocarbon receptor (AHR) is receptor found on epithelial cells of the gastrointestinal tract that is known to induce anti-inflammatory immune signaling in response to indole derivatives and other microbial metabolites (Postler et al., 2017). Utilizing an AHR reporter system activation of this signaling pathway is examined as previously described (Marinelli et al., 2018). Microbially conditioned supernatant is then incubated with HT29 human cells containing an AHR luciferase reporter system, wherein the luciferase gene is under the control of an AHR controlled promoter. Supernatant that stimulates AHR signaling induces expression of the luciferase reporter gene. Luciferase activity is measured spectroscopically to quantify AHR signaling induced by the supernatant.

Other immunomodulatory pathways that are examined are the Toll-Like Receptor (TLR) pathways. TLRs are immunological receptors that detect specific microbial molecular patterns. In response to stimulation by their ligands, these receptors can promote pro- and anti-inflammatory responses. Of specific interest are the TLR heterodimers TLR1/TLR2 and TLR2/TLR6 and the homodimers TLR4 and TLR5. It has been shown that the TLR2-based heterodimers can induce the production of the anti-inflammatory cytokine, IL-10, upon binding to microbial cell wall components (Cario E, 2004; Jang S, 2004; Saraiva M, 2010; Nguyen, 2020). Additionally, while TLR4 and TLR5 are often thought of as pro-inflammatory receptors, studies have indicated that TLR4 activation can lead to secretion of IL-10 resulting in the maturation of regulatory T cells to control the inflammatory response (Higgins S, 2003). Furthermore, TLR5 signaling is critical for maintenance of the epithelial barrier within the gastrointestinal tract. TLR5 activation by commensal bacteria has been shown to inhibit general inflammation within the gut. The loss of TLR5 expression from intestinal epithelial cells has been shown to increase inflammation and epithelial permeability within the gastrointestinal tract (Vijay-Kumar et al., 2007, Chassaing et al., 2015). Reporter cell lines, such as the HEK-Blue TLR5 reporter line are used to demonstrate TLR signaling in response to microbial ligands. Microbes are grown in pure culture. These microbes or microbially conditioned supernatant are used to treat human cells that contain a reporter gene has been placed under the control of a TLR controlled reporter. When the TLR becomes activated by a microbe or secreted microbial compound, the reporter gene is expressed producing a measurable result.

### Example 10: Altered Cytokine Responses from DMA-Treated Peripheral Blood Mononuclear Cells with and without Inflammatory Stimulation In Vitro

Microbial metabolites and components are known to influence immune cells, shaping immune responses through interactions with various cell types and secreted molecules like cytokines and chemokines. Given the complexity of immune responses, in vitro model systems are essential for studying immune cell functions. Peripheral blood mononuclear cells (PBMCs)—which include T cells, B cells, dendritic cells, NK cells, and monocytes—offer a versatile model for examining comprehensive immune responses, as they represent a diverse array of immune cells relevant to human immunity (Klieveland 2015). Aging, and menopause in particular, can lead to enhanced inflammatory signaling, which has been demonstrated in PBMCs isolated from post-menopausal women (Rachon et al., 2002; Paik et al., 2012).

To examine the influence of DMAs on immune cells in vitro, cryopreserved human PBMCs were procured or fresh human PBMCs were isolated from whole blood. These cells were treated with media controls, lipopolysaccharide (LPS, a stimulatory control), or a defined microbial assemblage (DMA) at varying multiplicities of interaction (MOI), the number of microbes applied per individual human cell). Cells were then incubated with their individual treatment conditions for 24 hours (Hua et al., 2010). Human cells and microbes were removed from solutions by centrifugation and cell viability was determined by secreted lactate dehydrogenase activity. PBMCs were treated with either DMAs at MOIs of 1 and 10, and cytokine secretion was examined via enzyme-linked immunosorbent assay (ELISA) to characterize the immune responses elicited by different DMAs. Quantified cytokines and chemokines included: interferon γ (IFNγ), transforming growth factor β (TGFβ), tumor necrosis factor α (TNFα), osteoprotegerin (OPG), CXCL-1, and interleukins (IL)-10, -12, -1β, -23, -6, -18, and -8 (Bartelt et al., 2009; Schildberger et al., 2013).

### Example 11: Examination of Barrier Integrity and Immune Response of Intestinal Epithelial Monolayers Treated with DMAs In Vitro

The intestinal epithelium is considered an active participant in host defense. It forms a physical barrier that segregates the host from potentially noxious luminal microbes and compounds but is also capable of secreting cytokines to recruit immune cells. The intestinal barrier contains an essential component of intercellular junction complexes, known as tight junctions. They are composed of junction proteins, such as occludin and claudin, that control the permeability of molecules in adjacent cells (Paradis et al., 2021). Complex interactions between the epithelial cells, mucus layer, and immune cells regulate the function of the intestinal barrier. When there is a disruption to these interactions, barrier dysfunction can occur. Certain probiotics such as Saccharomyces boulardii have been shown to improve barrier function (Czerucka et al., 2007). We sought to determine whether DMAs were capable of modulating intestinal barrier integrity and cytokine signaling. We investigated the capability of DMAs to modulate intestinal barrier integrity and influence cytokine signaling pathways.

Briefly, Caco-2 and HT-29 human epithelial cells were seeded in a 7:3 ratio on semipermeable membrane supports to generate a differentiated, polarized, epithelial monolayer. Cells were cultured in DMEM medium containing 10% FBS, 1 mM Glutamine, and 1× Anti-Anti (Gibco) at 37° C., 5% CO₂ for approximately 18 days to allow for tight junction formation. During this time, barrier integrity measurements were taken at regular intervals using transepithelial electrical resistance (TEER) (Srinivasan et al., 2015). Cells were then treated with a DMA at varying MOIs, E. coli, or media controls and co-incubated for 16-24 hours and TEER was again measured. Cells and microbes were removed from solutions by centrifugation. Cytokines (IL-8 and CXCL-1) were quantified by ELISA to determine how DMAs influenced immune responses.

### Example 12: Preclinical Validation of DMA Efficacy in Animal Models of Disease Aged Mice (Inflammaging)

DMAs are evaluated for their therapeutic efficacy in a mouse model of aging-associated inflammation. All mice are group housed with 5 mice per cage in individually ventilated cages (IVCs) specifically designed for germ free husbandry. After an acclimation period, baseline samples of feces and blood are collected, and baseline measures of body mass are recorded. After baseline measures are recorded, 18-month-old C57bl/6J male and female are randomly divided into groups and administered by bi-daily oral gavage of water (negative control), or one of five test DMAs for a period of 6-weeks. Bi-weekly fecal samples are collected to monitor the functional and taxonomic composition of the gut microbiome over time. 1-week prior to sacrifice, fasted animals receive an oral gavage of FITC-dextran, and a blood sample will be collected 4-hours later to measure gut permeability. After the 6-week administration period, tissues are collected from each mouse for downstream analysis as follows.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

Gut permeability analysis: Following FITC-dextran administration to fasted mice, blood will be retro-orbitally collected after 4 hours, and fluorescence intensity will be measured on fluorescence plates using an excitation wavelength of 493 nm and an emission wavelength of 518 nm as previously described (Thevaranjan et al., 2017).

Circulating pro- and anti-inflammatory cytokine analysis: Blood is collected from each mouse into EDTA treated tubes at the time of sacrifice, and plasma is separated from cells. Cells are saved for peripheral blood mononuclear cell (PB MC) analysis. Plasma samples are analyzed by ELISA or Multiplex assay for circulating inflammatory cytokine levels including but not limited to CRP, TNF-α, IL-1β, IL-2 IL-3, IL-4, IL-5, IL-6, IL-7, IL-8, IL-9, IL-10, IL-12, IL-13, IL-15, IL-17, IL-21, IL-22, IL-23, IL-27 IFNγ, G-CSF, G3M-CSF, IP-10, KC, LIF, LIX, MCP-1, M-CSF, MIG, MIP-1α, MIP-1β, MIP-2, MIP-3, RANTES, RANKL and VEGF as previously described (Schott et al., 2018).

Peripheral blood mononuclear cell (PBMIC) analysis: P BMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNγ and IL-1β. RNA is extracted and analyzed by qRT-PCR, and protein is extracted and evaluated by ELISA and/or western blot as previously described for the aforementioned proteins of interest (Li et al., 2016).

### Rheumatoid Arthritis

DMAs are evaluated for their therapeutic efficacy in a mouse model of rheumatoid arthritis (R A) called the collagen-induced arthritis model (CIA) as well as in a delayed type hypersensitivity model. For a prototypical CIA mouse model study, all mice are group housed with 3-5 mice per cage in individually ventilated cages (IVCs) specifically designed for germ free husbandry. Adult male and female DBA/1 mice are randomly allocated to experimental groups and allowed to acclimate for two weeks. After an acclimation period, baseline samples of feces and blood are collected, and baseline measures of body mass are recorded. On Day 0, animals are administered by subcutaneous injection with 100 microliters of an emulsion containing 100 micrograms of type II collagen (CII) in incomplete's Freund's adjuvant supplemented with 4 mg/ml Mycobacterium tuberculosis H37Ra. On Day 21, animals are administered by subcutaneous injection with a booster emulsion containing 100 μg of type II collagen in incomplete Freund's adjuvant Beginning from day −14 and continuing through day −45 (end of experiment), mice are administered by bi-daily oral gavage of water (negative control) or one of five test DMAs. From Day −14 until the end of the experiment on Day 45, animals are weighed three times per week. From Day 21 until the end of the experiment, animals are scored three times per week for clinical signs of arthritis to include swelling of the hind- and front paws, radio-carpal (wrist) joints and tibio-tarsal (ankle joints. At the end of the experiment on day 45, mice are euthanized, and tissues are collected from each mouse for downstream analysis as follows.

Circulating pro- and anti-inflammatory cytokine analysis: Blood is collected from each mouse into EDTA treated tubes at the time of sacrifice, and plasma is separated from cells. Cells are saved for peripheral blood mononuclear cell (PBMC) analysis. Plasma samples are analyzed by ELISA or Multiplex assay for circulating inflammatory cytokine levels including but not limited to CRP, TNF-α, IL-1β, IL-2, IL-3, IL-4, IL-5, IL-6, IL-7, IL-8, IL-9, IL-10, TL-12, IL-13, IL-15, TL-17, IL-21, IL-22, TL-23, IL-27 IFN, G-CSF, GM-CSF, IP-10, KC, LIF, LIX, MCP-1, M-CSF, MIG, MIP-1α, MIP-1β, MIP-2, MIP-3, RANTES, RANKL and VEGF, as previously described (Schott et al., 2018).

Peripheral blood mononuclear cell (PBMC) analysis: PBMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNγ and IL-1β. RNA is extracted and analyzed by qRT-PCR, and protein is extracted and evaluated by ELISA and/or western blot as previously described for the aforementioned proteins of interest (Li et al., 2016).

Metagenomic analysis of fecal pellets: DNA from the fecal pellets are extracted and the concentration are estimated. DNA libraries are prepared, and. raw sequencing reads are processed. Mouse DNA removal from the metagenomes is performed by mapping reads to the mouse reference genome GRCm38p6. Taxonomic classification of the short-read metagenomes are based on marker genes identified, and organism abundances are calculated at different classification levels (species, genus, family). Functional profiling of microbial community members is performed. The abundance of metabolic pathways in the gut microbiome are estimated using the HUMAnN2 output.

Histopathology: At the end of the experiment, hind paws are stored in tissue fixative. Samples are transferred into decalcification solution, and tissue samples are processed, sectioned, and stained with Haematoxylin & Eosin. Sections are scored by a qualified histopathologist, blind to the experimental design, for signs of arthritis to include inflammation, articular cartilage damage and damage to the underlying metaphyseal bone. A detailed scoring system is used (see Table 9). Data will be graphed (Mean±SEM). Raw and analyzed data will be provided as well as representative pictures.

TABLE 9
Histopathology scoring system

| Type | Grade | Description |
|---|---|---|
| Inflammation | 0 | Normal Joint |
| | 1 | Mild synovial hyperplasia with inflammation |
| | | dominated by neutrophils. Low numbers of |
| | | neutrophils and macrophages in joint space |
| | 2 | Synovial hyperplasia with moderate to marked |
| | | inflammation involving both neutrophils and |
| | | macrophages. Neutrophils and macrophages in |
| | | joint space; may be some necrotic tissue debris |
| | 3 | Synovial hyperplasia with marked inflammation |
| | | involving both neutrophils and macrophages. Loss |
| | | of synoviocyte lining Inflammation may extend |
| | | from synovium to surrounding tissue including |
| | | muscle. Numerous neutrophils and macrophages in |
| | | joint space, together with significant necrotic |
| | | tissue debris |
| Articular | 0 | Normal joint |
| cartilage | 1 | Articular cartilage shows only mild degenerative |
| damage | | change Early pannus formation may be present |
| | | peripherally. |
| | 2 | Articular cartilage shows moderate degenerative |
| | | change and focal loss. Pannus formation is present |
| | | focally |
| | 3 | Significant disruption and loss of articular cartilage |
| | | with extensive pannus formation |
| Damage | 0 | Normal joint |
| to the | 1 | No change to underlying metaphyseal bone |
| underlying | 2 | May be focal necrosis or fibrosis of metaphyseal |
| metaphyseal | | bone. |
| bone | 3 | Disruption or collapse of metaphyseal bone. |
| | | Extensive inflammation, necrosis or fibrosis |
| | | extending to medullary space of the metaphysis |

### Delayed Type Hypersensitivity Model of Inflammation

In addition to the collagen-induced arthritis mouse model of RA study described above, a delayed type hypersensitivity study for systemic inflammation is conducted in mice, and is conducted as follows. The studies are conducted in a BSL-1, quarantined room. Mice are acclimated to the facility for 1 week followed by an additional 2-week acclimation with bedding mixing to normalize microbiomes across cages. Fecal microbiome samples are collected 1-2 days prior to mBSA treatment #1. At 8 weeks of age (day 0), animals receive an intra-plantar mBSA (methylated Bovine Serum Albumin) challenge or PBS/Complete fruend's adjuvant (Control) in the right hind paw. At 8 weeks of age (day 0), immediately following mBSA, animals are treated with either DMAs (twice daily) or Dexamethasone (Dex) 5 mg/kg (once daily). Treatment with DMA or Dex is continued until day 8. Mice receiving DMAs are only gavaged in the morning on day 8. On day 8, mice receive an intra-plantar mBSA challenge or PBS/CFA (control) in the right hind paw after Dex or DMA treatment. Paw swelling is measured on day 9. At the end of the study, the spleen, blood plasma, mBSA injected paws, and fecal samples are collected and further characterized as described above. DMAs are identified that reduce paw inflammation and reduce pro-inflammatory cytokine secretion/detection in blood and injected paw samples.

### Imiquimod Model of Psoriasis

DMAs are evaluated for their therapeutic efficacy in a mouse model of psoriasis. All mice are group housed with up to 5 mice per cage in individually ventilated cages (IVCs) specifically designed for germ free husbandry. After an acclimation period, mice are randomly divided into groups, baseline samples of feces and blood are collected, and baseline measures of body mass are recorded. On day 7, mice begin bi-daily administration by oral gavage with water (negative control) or one of the test DMAs. On Day 0, psoriasis is induced in 8-10 week old C57B/6 male and female mice using the well described Imiquimod model (reviewed in Gangwar et al 2022). Briefly, topical dose of 62.5 mg IMQ cream (5% Aldara) is applied daily to the shaved backs and ears of mice. Control (CTL) animals are treated similarly with a non-toxic petrolatum or lanolin-derived occlusion cream. IMQ and CTL treatments are carried out for 5 consecutive days and mice are sacrificed on day 6. Erythema, scaling, and thickness are scored independently on a scale from 0-4: 0 representing no symptoms, 1 representing slight symptoms, 2 indicating moderate symptoms, 3 indicating marked symptoms, and 4 representing very marked symptoms. A cumulative score (scale: 0-12) is then calculated by adding the scores for all three symptoms. Fecal and serum samples are collected to monitor the functional and taxonomic composition of the gut and immune profile. After the 12-day administration period, tissues are collected from each mouse for downstream analysis as follows.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

Circulating pro- and anti-inflammatory cytokine analysis: Blood is collected from each mouse into EDTA treated tubes at the time of sacrifice, and plasma is separated from cells. Cells are saved for peripheral blood mononuclear cell (PBMC) analysis. Plasma samples are analyzed by ELISA or Multiplex assay for circulating inflammatory cytokine levels including but not limited to CRP, TNF-α, IL-1b, IL-2, IL-3, IL-4, IL-5, IL-6, IL-7, IL-8, IL-9, IL-10, IL-12, IL-13, IL-15, IL-17, IL-21, IL-22, IL-23, IL-27 IFNg, G-CSF, GM-CSF, IP-10, KC, LIF, LIX, MCP-1, M-CSF, MIG, MIP-1a, MIP-1b, MIP-2, MIP-3, RANTES, RANKL and VEGF as previously described (Schott et al., 2018).

Peripheral blood mononuclear cell (PBMC) analysis: PBMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNg and IL-1β. RNA is extracted and analyzed by qRT-PCR, and protein is extracted and evaluated by ELISA and/or western blot as previously described for the aforementioned proteins of interest (Li et al., 2016).

Histopathology: Skin tissue from the backs of the mice is collected and was fixed in 10% buffered formalin solution and embedded in paraffin. Sections (5-10 μm) are prepared and stained with hematoxylin and eosin (H&E). The tissue sections are histologically examined, and epidermal thickness is scored.

RNA, protein and cytokine analysis of dermal and splenic tissue: Tissue from backs, ears, and spleens are collected. RNA is extracted and analyzed by qRT-PCR as previously described (Glowacki et al., 2013), and protein is extracted and evaluated by ELISA and/or western blot as previously described for the cytokines and proteins of interest including but not limited to I IL-10, TGF-β, TNF-α, IL-17, and IL-23.

### Recombinant IL-23 Induced Psoriasis Model

DMAs are evaluated for their therapeutic efficacy in a mouse model of psoriasis. All mice are group housed with up to 5 mice per cage in individually ventilated cages (IVCs) specifically designed for germ free husbandry. After an acclimation period, mice are randomly divided into groups, baseline samples of feces and blood are collected, and baseline measures of ear thickness and body mass are recorded. On day 7, mice begin bi-daily administration by oral gavage with water (negative control) or one of the test DMAs. Starting on Day 0, psoriasis is induced in 7-week-old C57B/6 male and female mice using intradermally injected recombinant murine IL-23 (Rizzo et al 2011). Briefly, 1 μg rmIL-23 in PBS/0.1% BSA is injected into ears of mice. Control (CTL) animals are injected similarly with PBS/0.1% BSA. rIL-23 treatments are carried out on alternate days for 8-16 days and mice are sacrificed. Ear thickness is measured daily with calipers. The symptoms of erythema, scaling, and thickness are scored independently on a scale from 0-4: 0 representing no symptoms, 1 representing slight symptoms, 2 indicating moderate symptoms, 3 indicating marked symptoms, and 4 representing very marked symptoms. A cumulative score (scale: 0-12) is then calculated by adding the scores for all three symptoms. Fecal and serum samples are collected to monitor the functional and taxonomic composition of the gut and immune profile. After the 15-23-day administration period, tissues are collected from each mouse for downstream analysis as follows.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

Circulating pro- and anti-inflammatory cytokine analysis: Blood is collected from each mouse into EDTA treated tubes at the time of sacrifice, and plasma is separated from cells. Cells are saved for peripheral blood mononuclear cell (PBMC) analysis. Plasma samples are analyzed by ELISA or Multiplex assay for circulating inflammatory cytokine levels including but not limited to CRP, TNF-α, IL-1b, IL-2, IL-3, IL-4, IL-5, IL-6, IL-7, IL-8, IL-9, IL-10, IL-12, IL-13, IL-15, IL-17, IL-21, IL-22, IL-23, IL-27 IFNg, G-CSF, GM-CSF, IP-10, KC, LIF, LIX, MCP-1, M-CSF, MIG, MIP-1a, MIP-1b, MIP-2, MIP-3, RANTES, RANKL and VEGF as previously described (Schott et al., 2018).

Peripheral blood mononuclear cell (PBMC) analysis: PBMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNg and IL-1β. RNA is extracted and analyzed by qRT-PCR, and protein is extracted and evaluated by ELISA and/or western blot as previously described for the aforementioned proteins of interest (Li et al., 2016).

Histopathology: Skin tissue from the ears of the mice is collected and was fixed in 10% buffered formalin solution and embedded in paraffin. Sections (5-10 μm) are prepared and stained with hematoxylin and eosin (H&E). The tissue sections are histologically examined, and epidermal thickness is scored.

RNA, protein and cytokine analysis of dermal and splenic tissue: Tissue from ears, and spleens are collected. RNA is extracted and analyzed by qRT-PCR as previously described (Glowacki et al., 2013), and protein is extracted and evaluated by ELISA and/or western blot as previously described for the cytokines and proteins of interest including but not limited to I IL-10, TGF-β, TNF-α, IL-17, and IL-23.

### Psoriatic Arthritis

DMAs are evaluated for their therapeutic efficacy in a mouse model of psoriasis and psoriatic arthritis. All mice are group housed with up to 5 mice per cage in individually ventilated cages (IVCs) specifically designed for germ free husbandry. After an acclimation period, mice are randomly divided into groups, baseline samples of feces and blood are collected, and baseline measures of body mass are recorded. On day −7 mice begin bi-daily administration by oral gavage with water (negative control) or one of the test DMAs. On day 0 B10Q.Ncf1m1j/m1j mice are injected i.p. with 10 or 20 mg of mannan from the yeast, S. cerevisiae dissolved in 200 μL of PBS. Mice are scored daily for inflammation in the peripheral joints. The severity and incidence of the Ps-like skin manifestations are monitored on a scale ranging from 1 to 3 per mouse based on the severity of skin scaling on the paws: 1, weak skin peeling; 2, moderate skin peeling; and 3, heavy skin peeling with some hair loss. Weekly fecal and serum samples are collected to monitor the functional and taxonomic composition of the gut and immune profile over time. After the 14-day administration period, tissues are collected from each mouse for downstream analysis as follows.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

Circulating pro- and anti-inflammatory cytokine analysis: Blood is collected from each mouse into EDTA treated tubes at the time of sacrifice, and plasma is separated from cells. Cells are saved for peripheral blood mononuclear cell (PBMC) analysis. Plasma samples are analyzed by ELISA or Multiplex assay for circulating inflammatory cytokine levels including but not limited to CRP, TNF-α, IL-1b, IL-2, IL-3, IL-4, IL-5, IL-6, IL-7, IL-8, IL-9, IL-10, IL-12, IL-13, IL-15, IL-17, IL-21, IL-22, IL-23, IL-27 IFNg, G-CSF, GM-CSF, IP-10, KC, LIF, LIX, MCP-1, M-CSF, MIG, MIP-1a, MIP-1b, MIP-2, MIP-3, RANTES, RANKL and VEGF as previously described (Schott et al., 2018).

Peripheral blood mononuclear cell (PBMC) analysis: PBMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNg and IL-1β. RNA is extracted and analyzed by qRT-PCR, and protein is extracted and evaluated by ELISA and/or western blot as previously described for the aforementioned proteins of interest (Li et al., 2016).

Histopathology: Naive and diseased mouse paws and healthy and psoriatic/eczematic skin biopsies are fixed in 4% paraformaldehyde, decalcified (only mouse paws), and embedded in paraffin. The ear tissue is frozen and embedded, and 10-μm sections are stained with H&E for the assessment of inflammation. For the evaluation of arthritis activity and severity in the ankle joint sections, the following histopathological scoring protocol is used: 1, mild synovitis with hyperplastic synovial membrane, small focal infiltration of inflammatory cells, and increased numbers of vessels in the synovium with no bone or cartilage erosions; 2, moderate synovitis, entheseal inflammation, cartilage erosions, and undisrupted joint architecture; and 3, severe pannus formation with extensive erosions of bone and cartilage and disrupted joint architecture.

RNA, protein, and cytokine analysis of dermal, joint, and splenic tissue: Tissue from backs, ears, paws and spleens are collected. RNA is extracted and analyzed by qRT-PCR as previously described (Glowacki et al., 2013), and protein is extracted and evaluated by ELISA and/or western blot as previously described for the cytokines and proteins of interest including but not limited to IL-10, TGF-β, TNF-α, IL-17, and IL-23.

### IL-23 EEV Induced Psoriasis/Psoriatic Arthritis Model

DMAs are evaluated for their therapeutic efficacy in a mouse model of psoriasis and psoriatic arthritis. All mice are group housed with up to 5 mice per cage in individually ventilated cages (IVCs) specifically designed for germ free husbandry. After an acclimation period, mice are randomly divided into groups, baseline samples of feces and blood are collected, and baseline measures of body mass are recorded. On day −7 mice begin bi-daily administration by oral gavage with water (negative control) or one of the test DMAs. On day 0, 9-10 week old Male B10.RIII mice are given 75 ng IL-23 enhanced episomal vector (EEV) (System Biosciences, Palo Alto, CA, USA) or empty CAGS EEV by hydrodynamic delivery (HDD) (Mortier et al 2023). Mice are scored daily for inflammation in the peripheral joints for seven days. The severity and incidence of the Ps-like skin manifestations are monitored on a scale ranging from 1 to 3 per mouse based on the severity of skin scaling on the paws: 1, weak skin peeling; 2, moderate skin peeling; and 3, heavy skin peeling with some hair loss. Weekly fecal and serum samples are collected to monitor the functional and taxonomic composition of the gut and immune profile over time. After the 35-day administration period, tissues are collected from each mouse for downstream analysis as follows.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

Circulating pro- and anti-inflammatory cytokine analysis: Blood is collected from each mouse into EDTA treated tubes at the time of sacrifice, and plasma is separated from cells. Cells are saved for peripheral blood mononuclear cell (PBMC) analysis. Plasma samples are analyzed by ELISA or Multiplex assay for circulating inflammatory cytokine levels including but not limited to CRP, TNF-α, IL-1β, IL-2, IL-3, IL-4, IL-5, IL-6, IL-7, IL-8, IL-9, IL-10, IL-12, IL-13, IL-15, IL-17, IL-21, IL-22, IL-23, IL-27 IFNγ, G-CSF, GM-CSF, IP-10, KC, LIF, LIX, MCP-1, M-CSF, MIG, MIP-1α, MIP-1β, MIP-2, MIP-3, RANTES, RANKL and VEGF as previously described (Schott et al., 2018).

Peripheral blood mononuclear cell (PBMC) analysis: PBMC populations are analyzed by flow cytometry as previously described (Rao et al., 2012). Anticoagulant treated blood undergoes density gradient centrifugation in Ficol to isolate PBMC populations from granulocytes, erythrocytes, and platelets. After which, PBMCS are washed thoroughly and resuspended in FACS buffer. Following resuspension cells undergo Fc (Constant Fragment) blocking to obstruct antibody binding to cellular Fc receptors. Cell suspensions are then treated with unique fluorescent antibodies that are specific for immunophenotyping markers. For instance, CD3 antibodies and CD19 antibodies would be used to label T and B cells, respectively. Through this process T cells, T cell subsets, B cells, monocytes, dendritic cells, phagocyte subsets, natural killer cells and other cellular populations, as deemed relevant, are analyzed.

Bone marrow and splenic immune cell analysis: Bone marrow aspirates and spleens are collected for immune cell phenotyping by flow cytometry and RNAseq as previously described (Tyagi et al., 2018). Upon collection, samples are split in half and either flash frozen for RNA analysis or immediately processed for flow cytometry. Samples are processed for flow cytometry as described for PBMCs, with the addition that spleens are homogenized either mechanically or enzymatically prior to washing and resuspension in FACS buffer.

Colonic tissue isolation for RNA and protein analysis: Two cm sections of proximal colonic tissue and two cm sections of terminal ileum tissue are isolated, split in half, and flash frozen for RNA and protein evaluation of tight junctions and claudins, critical mediators of gut barrier integrity, as well as inflammatory cytokine levels including but not limited to TNF-α, IL-17, IFNγ and IL-1β. RNA is extracted and analyzed by qRT-PCR, and protein is extracted and evaluated by ELISA and/or western blot as previously described for the aforementioned proteins of interest (Li et al., 2016).

Histopathology: Naive and diseased mouse paws and healthy and psoriatic/eczematic skin biopsies are fixed in 4% paraformaldehyde, decalcified (only mouse paws), and embedded in paraffin. The ear tissue is frozen and embedded in Tissue Tec medium, and 10-μm sections are stained with H&E for the assessment of inflammation. For the evaluation of arthritis activity and severity in the ankle joint sections, the following histopathological scoring protocol is used: 1, mild synovitis with hyperplastic synovial membrane, small focal infiltration of inflammatory cells, and increased numbers of vessels in the synovium with no bone or cartilage erosions; 2, moderate synovitis, entheseal inflammation, cartilage erosions, and undisrupted joint architecture; and 3, severe pannus formation with extensive erosions of bone and cartilage and disrupted joint architecture.

RNA, protein, and cytokine analysis of dermal, joint, and splenic tissue: Tissue from backs, ears, paws and spleens are collected. RNA is extracted and analyzed by qRT-PCR as previously described (Glowacki et al., 2013), and protein is extracted and evaluated by ELISA and/or western blot as previously described for the cytokines and proteins of interest including but not limited to IL-10, TGF-β, TNF-α, IL-17, and IL-23.

H. pylori-Associated Gastritis

DMAs are evaluated for their therapeutic efficacy in a mouse model of Helicobacter pylori-associated gastritis. All mice are group housed with 5 mice per cage in individually ventilated cages (IVCs) specifically designed for germ free husbandry. Adult male and female C57Bl/6J mice are randomly allocated to experimental groups and allowed to acclimate for two weeks. After an acclimation period, baseline samples of feces and blood are collected, and baseline measures of body mass are recorded. On Day 0, animals are infected three times over a 5-day period with a 0.1 ml volume containing 10⁸ H. pylori (Sydney strain, SS1) organisms. Two weeks following infection, mice are treated by bi-daily oral gavage of water (negative control), triple antibiotic therapy of omeprazole, metronidazole, and clarithromycin (positive control), or one of five test DMAs for a period of 2-weeks. Fecal samples are collected weekly for metagenomic analysis. All animals are sacrificed 36 hours after the cessation of treatment for assessment of bacterial colonization by rapid qPCR and histology.

Histology. One-half of each stomach is placed into 10% buffered formalin and processed in paraffin, and 4-um sections will be stained with a modified Steiner silver stain. Colonization is assessed on a five-point scale: 0, no bacteria; 1, less than ⅓ of crypts colonized with 1 to 10 bacteria; 2, ⅓ to ⅔ of crypts colonized with 10 to 20 bacteria; 3, ⅔ of the crypts colonized with >20 bacteria; and 4, all crypts colonized with >20 bacteria as previously described (Velduyzen van Zanten et al., 2003).

Confirmation of H. pylori eradication by quantitative PCR: A longitudinal strip of gastric tissue from the greater curvature is digested with proteinase K at 55° C. overnight, followed by DNA extraction. H. pylori colonization levels in gastric tissue is quantified by PCR with strain specific primers as previously described (Velduyzen van Zanten et al., 2003). Any sample detecting <10 copies of the H. pylori genome is considered negative for H. pylori colonization.

Inflammatory cytokine quantification by qRTPCR: A longitudinal strip of gastric tissue from the greater curvature is isolated, and RNA is extracted and analyzed by qRT-PCR as previously described (Velduyzen van Zanten et al., 2003) to quantify inflammatory cytokines in the stomach tissue including but not limited to TNF-α, IL-1, and IFNγ.

Metagenomic analysis of fecal pellets: DNA from the fecal pellets are extracted and the concentration are estimated. DNA libraries are prepared and an equimolar volume of each library will be pooled and sequenced. Raw sequencing reads are processed. Mouse DNA removal from the metagenomes is performed by mapping reads to the mouse reference genome GRCm38p6.

Taxonomic classification of the short-read metagenomes are based on marker genes identified using MetaPhlAn2 (Segata et al., 2012), and organism abundances are calculated at different classification levels (species, genus, family). Functional profiling of microbial community members is performed using HUMAnN2 (Franzosa et al., 2018) and reference pathways databases including UniRef90 and ChocoPhlAn. The abundance of metabolic pathways in the gut microbiome are estimated using the HUMAnN2 output.

All references, issued patents and patent applications cited within the body of the instant specification are hereby incorporated by reference in their entirety, for all purposes.

### REFERENCES

Altschul et al. Basic local alignment search tool. J. Mol. Biol., 215:403-410 (1990)
Bansal, T., Alaniz, R. C., Wood, T. K., & Jayaraman, A. (2010). The bacterial signal indole increases epithelial-cell tight-junction resistance and attenuates indicators of inflammation. Proceedings of the National Academy of Sciences of the United States of America, 107(1), 228-233. https://doi.org/10.1073/pnas.0906112107
Bartelt, R. R., Cruz-Orcutt, N., Collins, M. & Houtman, J. C. D. Comparison of T cell receptor-induced proximal signaling and downstream functions in immortalized and primary T cells. PLoS One 4, e5430 (2009).
Briani C et al. Cobalamin Deficiency: Clinical Picture and Radiological Findings. Nutrients. 5:11, 4521-4539. (2013)
Caffaratti, C., Plazy, C., Mery, G., Tidjani, A. R., Fiorini, F., Thiroux, S., Toussaint, B., Hannani, D., & Le Gouellec, A. (2021). What We Know So Far about the Metabolite-Mediated Microbiota-Intestinal Immunity Dialogue and How to Hear the Sound of This Crosstalk. Metabolites, 11(6), 406. https://doi.org/10.3390/metabo11060406
Cario E. (2008). Barrier-protective function of intestinal epithelial Toll-like receptor 2. Mucosal immunology, 1 Suppl 1, S62-S66. https://doi.org/10.1038/mi.2008.47
Chamba, J.-F. & Perreard, É. Contribution of propionic acid bacteria to lipolysis of Emmental cheese. Lait 82, 33-44 (2002).
Chassaing, B., Ley, R. E., & Gewirtz, A. T. (2014). Intestinal epithelial cell toll-like receptor 5 regulates the intestinal microbiota to prevent low-grade inflammation and metabolic syndrome in mice. Gastroenterology, 147(6), 1363-77.e17. https://doi.org/10.1053/j.gastro.2014.08.033
Chen et al. Modulating AHR function offers exciting therapeutic potential in gut immunity and inflammation Cell & Bioscience 2023
Cheng, Q. et al. A Synergistic Indole-3-Acetic Acid-Producing Synthetic Bacterial Consortium Benefits Walnut Seedling Growth. Agronomy 14, 1657 (2024).
Cox et al. SolexaQA: At-a-glance quality assessment of Illumina second-generation sequencing data. BMC Bioinformatics. 11:485 (2010)
Czerucka, D., Piche, T. & Rampal, P. Review article: yeast as probiotics—Saccharomyces boulardii. Alimentary Pharmacology & Therapeutics 26, 767-778 (2007).
D'Aimmo M R., et. al. The potential of Bifidobacteria as a source of natural folate. Journal of Applied Microbiology. 112:975-984 (2012).
Das et al. Metagenomic analysis of microbe-mediated vitamin metabolism in the human gut microbiome. BMC Genomics. 20(1):208 (2019)
Denison, M. S., Pandini, A., Nagy, S. R., Baldwin, E. P. & Bonati, L. Ligand binding and activation of the Ah receptor. Chem Biol Interact 141, 3-24 (2002).
DiVincenzo et al. Gut microbiota, intestinal permeability, and systemic inflammation: a narrative review IEM 2023
Fasano, A. All disease begins in the (leaky) gut: role of zonulin-mediated gut permeability in the pathogenesis of some chronic inflammatory diseases. F1000Res 9, F1000 Faculty Rev-69 (2020)
Fang H. et al. Microbial production of vitamin B12: a review and future perspectives. Microb Cell Fact. 16(1):15 (2017)
Franzosa et al. Species-level functional profiling of metagenomes and metatranscriptomes Nat. Methods. 15. 962-968 (2018)
Gao, J., Xu, K., Liu, H., Liu, G., Bai, M., Peng, C., Li, T., & Yin, Y. (2018). Impact of the Gut Microbiota on Intestinal Immunity Mediated by Tryptophan Metabolism. Frontiers in cellular and infection microbiology, 8, 13. https://doi.org/10.3389/fcimb2018.00013
Gangwar, R. S., Gudjonsson, J. E., Ward, N. L. (2022). Mouse Models of Psoriasis: A Comprehensive Review. J Invest Dermatol 142, 884-897. https://doi.org/10.1016/j.jid.2021.06.019
Ghosh et al. Intestinal Barrier Dysfunction, LPS Translocation, and Disease Development Journal of the Endocrine Society. 2020
Ghosh, S., et al., Regulation of Intestinal Barrier Function by Microbial Metabolites. Cell Mol Gastroenterol Hepatol 11, 1463-1482 (2021)
Glowacki, A. J., Yoshizawa, S., Jhunjhunwala, S., Vieira, A. E., Garlet, G. P., Sfeir, C., & Little, S. R. (2013). Prevention of inflammation-mediated bone loss in murine and canine periodontal disease via recruitment of regulatory lymphocytes. Proceedings of the National Academy of Sciences of the United States of America, 110(46), 18525-18530. https://doi.org/10.1073/pnas.1302829110
Godoy-Vitorino, F. Insights into Gut Dysbiosis: Inflammatory Diseases, Obesity, and Restoration Approaches. MDPI, 2024
The Handbook of Pharmaceutical Excipients. 6^{th} edition. American Pharmaceutical Association (1986)
Higgins, S. C., Lavelle, E. C., McCann, C., Keogh, B., McNeela, E., Byrne, P., O'Gorman, B., Jarnicki, A., McGuirk, P., & Mills, K. H. (2003). Toll-like receptor 4-mediated innate IL-10 activates antigen-specific regulatory T cells and confers resistance to Bordetella pertussis by inhibiting inflammatory pathology. Journal of immunology (Baltimore, Md.: 1950), 171(6), 3119-3127. https://doi.org/10.4049/jimmunol.171.6.3119
Hua, M.-C. et al. Probiotic Bio-Three induces Th1 and anti-inflammatory effects in PBMC and dendritic cells. World J Gastroenterol 16, 3529-3540 (2010).
Issac I, et al. Vitamin B12 Deficiency: An Important Reversible Co-Morbidity in Neuropsychiatric Manifestations. Indian Journal of Psychiatric Medicine. 37:1, 26-29. (2015)
Jang, S., Uematsu, S., Akira, S., & Salgame, P. (2004). IL-6 and IL-10 induction from dendritic cells in response to Mycobacterium tuberculosis is predominantly dependent on TLR2-mediated recognition. Journal of immunology (Baltimore, Md.: 1950), 173(5), 3392-3397. https://doi.org/10.4049/jimmunol.173.5.3392
Kim et al. FMAP: Functional Mapping and Analysis Pipeline for metagenomics and metatranscriptomics studies. BMC Bioinf 17:420 (2016)
Kimura, I., Ichimura, A., Ohue-Kitano, R. & Igarashi, M. Free Fatty Acid Receptors in Health and Disease. Physiological Reviews 100, 171-210 (2020).
Kleiveland, C. R. Peripheral Blood Mononuclear Cells. in The Impact of Food Bioactives on Health: in vitro and ex vivo models (eds. Verhoeckx, K. et al.) (Springer, Cham (CH), 2015)
Kok D, et al. Bacterial folate biosynthesis and colorectal cancer risk: more than just a gut feeling. Critical Reviews in Food Science and Nutrition. 60:2, 244-256 (2018)
Laiño J E., et. al. Applicability of a Lactobacillus amylovorus strain as a co-culture for natural folate bio-enrichment of fermented milk. International Journal of Food Microbiology. 191:10-16. (2014).
Lawenius, L. et al. Development of a synbiotic that protects against ovariectomy-induced trabecular bone loss. American Journal of Physiology-Endocrinology and Metabolism 322, E344-E354 (2022).
Li, P., Gu, Q., Zhou, Q. Complete genome sequence of Lactobacillus plantarum LZ206, a potential probiotic strain with antimicrobial activity against food-borne pathogenic microorganisms. Journal of Biotechnology. 238:(52-55). (2016).
Li, J. Y., Chassaing, B., Tyagi, A. M., Vaccaro, C., Luo, T., Adams, J., Darby, T. M., Weitzmann, M. N., Mulle, J. G., Gewirtz, A. T., Jones, R. M., & Pacifici, R. (2016). Sex steroid deficiency-associated bone loss is microbiota dependent and prevented by probiotics. The Journal of clinical investigation, 126(6), 2049-2063. https://doi.org/10.1172/JCI86062
Liu, P. et al. The role of short-chain fatty acids in intestinal barrier function, inflammation, oxidative stress, and colonic carcinogenesis. Pharmacological Research 165, 105420 (2021).
Macfarlane, S., & Macfarlane, G. T. (2003). Regulation of short-chain fatty acid production. The Proceedings of the Nutrition Society, 62(1), 67-72. https://doi.org/10.1079/PNS2002207
Magnusdottir et al. Generation of genome-scale metabolic reconstructions for 773 members of the human gut microbiota. Nat. Biotech. 35, 81-8 (2016)
Mann et al Short-chain fatty acids: linking diet, the microbiome and immunity Nature Reviews Immunology 2024
Marinelli, L., Martin-Gallausiaux, C., Bourhis, J M. et al. Identification of the novel role of butyrate as AhR ligand in human intestinal epithelial cells. Sci Rep 9, 643 (2019). https://doi.org/10.1038/s41598-018-37019-2
Mortier, C., Gracey, E., Coudenys, J., Manuello, T., Decruy, T., Maelegheer, M., Stappers, F., Gilis, E., Gaublomme, D., Van Hoorebeke, L., Van Welden, S., Ambler, C., Hegen, M., Symanowicz, P., Steyn, S., Berstein, G., Elewaut, D., Venken, K. (2023). RORγt inhibition ameliorates IL-23 driven experimental psoriatic arthritis by predominantly modulating γδ-T cells. Rheumatology, kead022.
Needleman and Wunsch. A General Method Applicable to the Search for Similarities in the Amino Acid Sequence of Two Proteins. J. Mol. Biol. 48:443 (1970)
Nguyen, B. N., Chávez-Arroyo, A., Cheng, M. I., Krasilnikov, M., Louie, A., & Portnoy, D. A. (2020). TLR2 and endosomal TLR-mediated secretion of IL-10 and immune suppression in response to phagosome-confined Listeria monocytogenes. PLoS pathogens, 16(7), e1008622. https://doi.org/10.1371/journal.ppat.1008622
Paik, J. K. et al. Circulating and PBMC Lp-PLA2 Associate Differently with Oxidative Stress and Subclinical Inflammation in Nonobese Women (Menopausal Status). PLOS ONE 7, e29675 (2012).
Paradis, T., Bègue, H., Basmaciyan, L., Dalle, F. & Bon, F. Tight Junctions as a Key for Pathogens Invasion in Intestinal Epithelial Cells. International Journal of Molecular Sciences 22, 2506 (2021).
Pearson and Lipman. Improved Tools for Biological Sequence Comparison. Proc. Nat'l. Acad. Sci. USA 85:2444 (1988)
Peng Y., et al. IDBA—A Practical Iterative de Bruijn Graph De Novo Assembler. Lisbon: RECOMB; (2010)
Postler, T. S., & Ghosh, S. (2017). Understanding the Holobiont: How Microbial Metabolites Affect Human Health and Shape the Immune System. Cell metabolism, 26(1), 110-130. https://doi.org/10.1016/j.cmet.2017.05.008
Rachon, D., Mysliwska, J., Suchecka-Rachon, K., Wieckiewicz, J. & Mysliwski, A. Effects of oestrogen deprivation on interleukin-6 production by peripheral blood mononuclear cells of postmenopausal women. (2002) doi:10.1677/joe.0.1720387.
Rathbone et al. Modified-Release Drug Delivery Technology (Drugs and the Pharmaceutical Sciences Book 184) 2nd Edition, Marcel Dekker, Inc. New York, N.Y. (2002).
Rao, S. P., Sancho, J., Campos-Rivera, J., Boutin, P. M., Severy, P. B., Weeden, T., Shankara, S., Roberts, B. L., & Kaplan, J. M. (2012). Human peripheral blood mononuclear cells exhibit heterogeneous CD52 expression levels and show differential sensitivity to alemtuzumab mediated cytolysis. PloS one, 7(6), e39416.
Remington's pharmaceutical sciences, 17th edition. Edited by Alfonso R. Gennaro. Mack Publishing Co., 20th and Northampton Streets, Easton, PA 18042 (1985)
Rizzo, H. L., Kagami, S., Phillips, K. G., Kurtz, S. E., Jacques, S. L., Blauvelt, A. (2011). IL-23-mediated psoriasis-like epidermal hyperplasia is dependent on IL-17A. J Immunol, 186, 1495-1502.
Salimi, F., Farrokh, P. Recent advances in the biological activities of microbial exopolysaccharides. World J Microbiol Biotechnol 2023
Saraiva, M., & O'Garra, A. (2010). The regulation of IL-10 production by immune cells. Nature reviews. Immunology, 10(3), 170-181.
Schildberger, A., Rossmanith, E., Eichhorn, T., Strassl, K. & Weber, V. Monocytes, peripheral blood mononuclear cells, and THP-1 cells exhibit different cytokine expression patterns following stimulation with lipopolysaccharide. Mediators Inflamm 2013, 697972 (2013).
Schlatterer, K., Peschel, A. & Kretschmer, D. Short-Chain Fatty Acid and FFAR2 Activation—A New Option for Treating Infections? Front. Cell. Infect. Microbiol. 11, (2021).
Schoch C L, et al. Nuclear ribosomal internal transcribed spacer (ITS) region as a universal DNA barcode marker for Fungi. Proc Natl Acad Sci USA 109:6241-6246 (2012)
Schott, E. M., Farnsworth, C. W., Grier, A., Lillis, J. A., Soniwala, S., Dadourian, G. H., Bell, R. D., Doolittle, M. L., Villani, D. A., Awad, H., Ketz, J. P., Kamal, F., Ackert-Bicknell, C., Ashton, J. M., Gill, S. R., Mooney, R. A., & Zuscik, M. J. (2018). Targeting the gut microbiome to treat the osteoarthritis of obesity. JCI insight, 3(8), e95997.
Scortichini, S., Boarelli, M. C., Silvi, S., & Fiorini, D. (2020). Development and validation of a GC-FID method for the analysis of short chain fatty acids in rat and human faeces and in fermentation fluids. Journal of chromatography. B, Analytical technologies in the biomedical and life sciences, 1143, 121972. https://doi.org/10.1016/j.jchromb.2020.121972
Segata, N., Waldron, L., Ballarini, A., Narasimhan, V., Jousson, O., & Huttenhower, C. (2012). Metagenomic microbial community profiling using unique clade-specific marker genes. Nature methods, 9(8), 811-814. https://doi.org/10.1038/nmeth.2066
Sethi, S., & Samantaray, U. S. (2021). Isolation and biochemical characterization of indole-3-acetic acid (IAA) produced in Pseudomonas sp. isolated from rhizospheric soil. International Journal for Research in Applied Sciences and Biotechnology, 8(4). https://doi.org/10.31033/ijrasb.8.4.1
Smith and Waterman. Comparison of biosequences. Adv. Appl. Math. 2:482 (1981)
Srinivasan, B. et al. TEER Measurement Techniques for In Vitro Barrier Model Systems. SLAS Technology 20, 107-126 (2015).
Strandwitz P. Neurotransmitter modulation by the gut microbiota. Brain Res. 2018 Aug. 15; 1693(Pt B):128-133
Sugahara H., et. al. Probiotic Bifidobacterium longum alters gut luminal metabolism through modification of the gut microbial community. Scientific Reports. 13548 (2015).
Terry P, et al. Dietary intake of folic acid and colorectal cancer risk in a cohort of women. International Journal of Cancer. 97:6, 864-867 (2002)
Thevaranjan, N., Puchta, A., Schulz, C., Naidoo, A., Szamosi, J. C., Verschoor, C. P., Loukov, D., Schenck, L. P., Jury, J., Foley, K. P., Schertzer, J. D., Larche, M. J., Davidson, D. J., Verdn, E. F., Surette, M. G., & Bowdish, D. (2017). Age-Associated Microbial Dysbiosis Promotes Intestinal Permeability, Systemic Inflammation, and Macrophage Dysfunction. Cell host & microbe, 21(4), 455-466.e4.
Truong et al. MetaPhlAn2 for enhanced metagenomic taxonomic profiling. Nature Methods. 12:10 (2015)
Tyagi, A. M., Yu, M., Darby, T. M., Vaccaro, C., Li, J. Y., Owens, J. A., Hsu, E., Adams, J., Weitzmann, M. N., Jones, R. M., & Pacifici, R. (2018). The Microbial Metabolite Butyrate Stimulates Bone Formation via T Regulatory Cell-Mediated Regulation of WNT10B Expression. Immunity, 49(6), 1116-1131.e7.
Velduyzen van Zanten, S. J., Kolesnikow, T., Leung, V., O'Rourke, J. L., & Lee, A. (2003). Gastric transitional zones, areas where Helicobacter treatment fails: results of a treatment trial using the Sydney strain mouse model. Antimicrobial agents and chemotherapy, 47(7), 2249-2255.
Vijay-Kumar, M. et al. Deletion of TLR5 results in spontaneous colitis in mice. J Clin Invest 117, 3909-3921 (2007).
Yin et al. dbCAN: a web resource for automated carbohydrate-active enzyme annotation. Nucleic Acids Res. 40 (2012)
Zhang et al. dbCAN2: a meta server for automated carbohydrate-active enzyme annotation. Nucleic Acids Res. 46(W1):W95-W101 (2018)

What is claimed is:

1. A synthetic microbial consortium, comprising:
i. at least 1×10\hat{ }7 colony forming units of a first microbial entity comprising a probiotic microbial species, and
ii. at least 1×10\hat{ }7 colony forming units of a second microbial entity comprising a food-derived microbial isolate,
wherein the synthetic microbial consortium produces a synergistic functional interaction when grown together relative to each distinct microbial entity grown in isolation under the same conditions.

2. The synthetic microbial consortium of claim 1, wherein the first microbial entity is selected from the group consisting of: a Bacillus subtilis, a Lactiplantibacillus plantarum, a Lactobacillus acidophilus, a Lacticaseibacillus casei, a Lacticaseibacillus rhamnosus, a Lacticaseibacillus paracasei, a Lentilactobacillus buchneri, a Levilactobacillus brevis, a Bacillus licheniformis, a Weizmannia coagulans, a Saccharomyces cerevisiae, and a Saccharomyces boulardii.

3. The synthetic microbial consortium of claim 1, wherein the second microbial entity is selected from the group consisting of: Bacillus subtilis, Lactiplantibacillus plantarum, Bacillus endophyticus, Bacillus amyloliquefaciens, Bacillus safensis, Bacillus pumilus, Bacillus paralicheniformis, Bacillus amyloliquefaciens, Bacillus axarquiensis, Bacillus megaterium, Bacillus aryabhattai, Pichia membranifaciens, Pichia kudriavzevii, Pichia terricola, Saccharomyces cerevisiae, Saccharomyces boulardii, Kluyveromyces marxianus, Talaromyces atroroseus, Debaryomyces hansenii, Candida akabanensis, Candida dosseyi, Meyerozyma guilliermondii, Lentilactobacillus buchneri, Lacticaseibacillus casei, Lactobacillus acidophilus, Leuconostoc mesenteroides, Leuconostoc pseudomesenteroides, Schleiferilactobacillus harbinensis, Pediococcus parvulus, Pediococcus ethanolidurans, Pediococcus pentosaceus, Pediococcus paracasei, Galactomyces geotrichum, Nakazawaea ishiwadae, Streptococcus thermophilus, Weisella cibaria, Kazachstania servazzii, Pichia fermentans, and Holermanniella takashimae.

4. The synthetic microbial consortium of claim 1, wherein the synergistic functional interaction results in a two-fold or greater increased number of colony forming units produced of the first microbial entity alone or of the first and second microbial entities combined when the microbial entities are grown together, relative to the summed amount of colony forming units produced by growing an equivalent amount of each distinct microbial entity in isolation under the same culture conditions, and wherein:
a. the first microbial entity is Bacillus subtilis, and the second microbial entity is selected from the group consisting of: Lentilactobacillus buchneri, Pediococcus parvulus, Lacticaseibacillus casei, Pediococcus ethanolidurans, Leuconostoc mesenteroides, Lactiplantibacillus plantarum, Schleiferilactobacillus harbinensis, Pichia membranifaciens, Pichia kudriavzevii, Meyerozyma guilliermondii, Holermanniella takashimae, Lactobacillus acidophilus, Galactomyces geotrichum, Candida akabanensis, Nakazawaea ishiwadae, and Saccharomyces cerevisiae;
b. the first microbial entity is Lacticaseibacillus casei, and the second microbial entity is selected from the group consisting of: Bacillus endophyticus, Bacillus amyloliquefaciens, Bacillus subtilis, Pichia membranifaciens, Pichia kudriavzevii, Pichia fermentans, Bacillus safensis, Bacillus pumilus, and Bacillus paralicheniformis;
c. the first microbial entity is Lactiplantibacillus plantarum, and the second microbial entity is selected from the group consisting of: Bacillus safensis, Bacillus subtilis, Saccharomyces cerevisiae, Pichia kudriavzevii, Saccharomyces boulardii, and Kluyveromyces marxianus;
d. the first microbial entity is Lacticaseibacillus paracasei, and the second microbial entity is selected from Kluyveromyces marxianus and Saccharomyces boulardii;
e. the first microbial entity is Lacticaseibacillus rhamnosus, and the second microbial entity is Kluyveromyces marxianus;
f. the first microbial entity is Lentilactobacillus buchneri, and the second microbial entity is selected from the group consisting of: Bacillus subtilis, Bacillus amyloliquefaciens, Bacillus amyloliquefaciens, Bacillus safensis, Bacillus axarquiensis, Talaromyces atroroseus, Kluyveromyces marxianus and Saccharomyces boulardii;
g. the first microbial entity is Lentilactobacillus acidophilus, and the second microbial entity is selected from the group consisting of Bacillus axarquiensis, Bacillus amyloliquefaciens, Bacillus subtilis, and Saccharomyces boulardii;
h. the first microbial entity is Levilactobacillus brevis, and the second microbial entity is Holermanniella takashimae;
i. the first microbial entity is Bacillus licheniformis, and the second microbial entity is Schleiferilactobacillus harbinensis or Pediococcus ethanolidurans;
j. the first microbial entity is Saccharomyces cerevisiae, and the second microbial entity is selected from the group consisting of: Bacillus pumilus, Bacillus subtilis, Bacillus amyloliquefaciens Lactiplantibacillus plantarum, and Leuconostoc mesenteroides;
or
k. the first microbial entity is Saccharomyces boulardii, and the second microbial entity is selected from the group consisting of: Lentilactobacillus buchneri, Pediococcus ethanolidurans, Lactobacillus harbinensis, Lactiplantibacillus plantarum, Lactobacillus acidophilus, Exiguobacterium sp., Pediococcus paracasei, Pediococcus pentosaceus and Lacticaseibacillus paracasei.

5. The synthetic microbial consortium of claim 1, wherein the synergistic functional interaction is production of an increased amount of acetate when the microbial entities are grown together, relative to the summed amount of acetate produced by an equivalent amount of each microbial entity grown in isolation under the same conditions, and wherein:
a. the first microbial entity is Bacillus subtilis, and the second microbial entity is selected from the group consisting of: Leuconostoc pseudomesenteroides, Saccharomyces boulardii, Pediococcus ethanolidurans, Pediococcus pentosaceus, Lactiplantibacillus plantarum, Pichia membranifaciens, Pichia fermentans, Talaromyces atroroseus, Debaryomyces hansenii, Candida dosseyi, Streptococcus thermophilus, and Weisella cibaria;
b. the first microbial entity is Lacticaseibacillus casei, and the second microbial entity is selected from the group consisting of: Pichia membranifaciens, Pichia fermentans, Pichia kudriavzevii, Bacillus atrophaeus, and Bacillus licheniformis;
c. the first microbial entity is Lacticaseibacillus paracasei, and the second microbial entity is selected from the group consisting of: Bacillus subtilis, Bacillus amyloliquefaciens, Kluyveromyces marxianus, Bacillus atrophaeus, and Saccharomyces boulardii;
d. the first microbial entity is Lactiplantibacillus plantarum, and the second microbial entity is selected from the group consisting of: Saccharomyces bouldardii, Kluyveromyces marxianus, Bacillus subtilis, Holermanniella takashimae, Bacillus pumilus, Talaromyces atroroseus, and Pichia terricola;
e. the first microbial entity is Lentilactobacillus buchneri, and the second microbial entity is Saccharomyces boulardii or Kluyveromyces marxianus;
f. the first microbial entity is Lentilactobacillus acidophilus, and the second microbial entity is Talaramyces atroroseus or Bacillus amyloliquefaciens;
g. the first microbial entity is Lacticaseibacillus rhamnosus, and the second microbial entity is Saccharomyces boulardii or Talaromyces atroroseus;
h. the first microbial entity is Bacillus licheniformis, and the second microbial entity is Lacticaseibacillus casei;
i. the first microbial entity is Weizmannia coagulans, and the second microbial entity is Pichia fermentans or Saccharomyces cerevisiae;
j. the first microbial entity is Saccharomyces cerevisiae, and the second microbial entity is Lactococcus lactis or Bacillus amyloliquefaciens;
or
k. the first microbial entity is Saccharomyces boulardii, and the second microbial entity is selected from the group consisting of: Lentilactobacillus buchneri, Pediococcus ethanolidurans, Lacticaseibacillus rhamnosus, Pediococcus ethanolidurans, Lactiplantibacillus plantarum, Bacillus amyloliquefaciens, Bacillus subtilis, Bacillus amyloliquefaciens, and Bacillus aryabhattai.

6. The synthetic microbial consortium of claim 1, wherein the synergistic functional interaction is production of an increased amount of propionate when the microbial entities are grown together, relative to the summed amount of propionate produced by an equivalent amount of each microbial entity grown in isolation under the same conditions, and wherein:
a. the first microbial entity is Bacillus subtilis, and the second microbial entity is selected from the group consisting of: Pediococcus pentosaceus, Pichia membranifaciens, Holermanniella takashimae, Lacticaseibacillus casei, Lentilactobacillus buchneri, Lacticaseibacillus casei, and Debaryomyces hansenii;
b. the first microbial entity is Lacticaseibacillus casei, and the second microbial entity is Bacillus subtilis or Bacillus safensis;
c. the first microbial entity is Lentilactobacillus buchneri, and the second microbial entity is Bacillus subtilis or Bacillus amyloliquefaciens;
or
d. the first microbial entity is Saccharomyces boulardii, and the second microbial entity is Bacillus amyloliquefaciens.

7. The synthetic microbial consortium of claim 1, wherein the synergistic functional interaction is production of an increased amount of indole derivatives when the microbial entities are grown together, relative to the summed amount of indole derivatives produced by an equivalent amount of each microbial entity grown in isolation under the same conditions, and wherein:
a. the first microbial entity is Bacillus subtilis, and the second microbial entity is selected from the group consisting of: Kazachstania servazzii, Kluyveromyces marxianus, Leuconostoc mesenteroides, Pediococcus parvulus, Leuconostoc pseudomesenteroides, Lactiplantibacillus plantarum, Lactiplantibacillus casei, Lentilactobacillus buchneri, Pediococcus pentosaceus, Pichia fermentans, Pichia kudriavzevii, Galactomyces geotrichum, Clavispora sp., Holermanniella takashimae, Talaromyces atroroseus, Weisella cibaria, Debaryomyces hansenii, Candida dosseyi, Saccharomyces cerevisiae, Candida almanaticensis, and Nakazawaea ishiwadae;
b. the first microbial entity is Lacticaseibacillus casei, and the second microbial entity is selected from the group consisting of: Bacillus licheniformis, Bacillus paralicheniformis, Bacillus endophyticus, and Bacillus subtilis;
c. the first microbial entity is Lactiplantibacillus plantarum, and the second microbial entity is selected from the group consisting of: Bacillus licheniformis, Bacillus paralicheniformis, Bacillus mojavensis, and Bacillus subtilis;
d. the first microbial entity is Lentilactobacillus buchneri, and the second microbial entity is Bacillus axarquiensis or Bacillus subtilis;
e. the first microbial entity is Lentilactobacillus acidophilus, and the second microbial entity is selected from the group consisting of Candida akabanensis, Pichia terricola, Debaryomyces hansenii, and Candida dosseyi;
f. the first microbial entity is Levilactobacillus brevis, and the second microbial entity is Bacillus subtilis;
g. the first microbial entity is Bacillus licheniformis, and the second microbial entity is Lacticaseibacillus casei or Lactiplantibacillus plantarum;
or
h. the first microbial entity is Saccharomyces cerevisiae, and the second microbial entity is selected from the group consisting of Bacillus mojavensis, Bacillus subtilis, Bacillus amyloliquefaciens, and Bacillus megaterium.

8. The synthetic microbial consortium of claim 1, further comprising a prebiotic polysaccharide.

9. The synthetic microbial consortium of claim 8, wherein the prebiotic polysaccharide is oligofructose, fructooligosaccharide, or a plant extract.

10. The synthetic microbial consortium of claim 1, further comprising a cryoprotectant present in an effective amount to extend survival of the microbial entities during storage at a cryogenic temperature.

11. The synthetic microbial consortium of claim 1, wherein the synthetic microbial consortium is formulated as a dietary supplement.

12. The synthetic microbial consortium of claim 1, wherein the synthetic microbial consortium is formulated as a solid foodstuff.

13. The synthetic microbial consortium of claim 1, wherein the synthetic microbial consortium is formulated as a medical food.

14. The synthetic microbial consortium of claim 1, wherein the synthetic microbial consortium is formulated as a pharmaceutical composition.

15. The synthetic microbial consortium of claim 1, wherein the microbial entities are co-formulated in a unit dose formulated for oral administration.

16. The synthetic microbial consortium of claim 15, wherein the unit dose comprises between 1×10⁷ and 1×10¹² cfu/dose of each of the first microbial entity and the second microbial entity.

17. The synthetic microbial consortium of claim 1, wherein the first microbial entity and the second microbial entity increase immune health or produce an anti-inflammatory effect in a mammalian host.

18. A method for treating, preventing, reducing severity, or enabling dietary management of at least one symptom in a subject having an immune system disorder, comprising: administering an effective amount of the synthetic microbial consortium of claim 1 to the subject, thereby treating, preventing, reducing the severity, or enabling the dietary management of the at least one symptom in the subject.

19. The method of claim 18, wherein the immune system disorder is selected from allergic rhinitis, allergic conjunctivitis, allergic bronchial asthma, atopic eczema, anaphylaxis, insect sting, drug allergy, food allergy, asthma, eczema, ulcerative colitis, Crohn's disease, celiac disease, multiple sclerosis, psoriasis, psoriatic arthritis, and a disorder or condition associated with a pathological Th17 activity.

20. A synthetic microbial consortium, comprising:
i. a probiotic Bacillus subtilis, and
ii. a second microbial entity comprising a food-derived microbial isolate selected from the group consisting of a Saccharomyces cerevisiae, a Lacticaseibacillus casei, a Pediococcus ethanolidurans, a Pichia membranifaciens, and a Lactiplantibacillus plantarum,
wherein the synthetic microbial consortium produces a synergistic functional interaction comprising production of an increased amount of acetate when the microbial entities are grown together, relative to the summed amount of acetate produced by an equivalent amount of each microbial entity grown in isolation under the same conditions,
and wherein administering an effective dose of the synthetic microbial consortium to a mammalian subject produces an anti-inflammatory effect in a mammalian host.
